(19) United States
(12) Patent Application Publication
(10) Pub. No.: US 2026/0152593 A1
(43) Pub. Date: Jun. 4, 2026

(54) MULTI-FUNCTIONAL POLYMERIZABLE COMPOUNDS AND METHODS OF PRODUCING AND USING THE SAME

(71) Applicant: Align Technology, Inc., San Jose, CA (US)

(72) Inventors: Jessica Kalay Su, San Jose, CA (US); Justin Nathan Wong, San Francisco, CA (US); Umesh Upendra Choudhary, Scotts Valley, CA (US)

(21) Appl. No.: 19/352,739

(22) Filed: Oct. 8, 2025

Related U.S. Application Data
(60) Provisional application No. 63/705,134, filed on Oct. 9, 2024.

(51) Int. Cl. C08F 290/06; A61C 7/00; A61C 7/08; B29C 64/124; B29C 64/188; B29K 33/04; B33Y 10/00; B33Y 30/00; B33Y 40/20; C07C 67/26; C08F 2/50

(52) U.S. Cl. CPC C08F 290/067; A61C 7/002; A61C 7/08; B29C 64/124; B29C 64/188; B33Y 10/00; B33Y 30/00; B33Y 40/20; C07C 67/26; C08F 2/50; B29K 2033/04; B29K 2995/0037

(57) ABSTRACT

The present disclosure provides synthesizing multi-functional polymerizable compounds and methods for the synthesizing the same. The methods include reacting a compound of Formula (Ia) with an epoxide of Formula (Ib) with quantitative conversion, requiring only an aqueous work-up for purification. Further provided herein are methods of producing polymerizable composition, methods of forming an article by additive manufacturing processes (e.g., via 3D printing), such as orthodontic appliances with increased stain resistance.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE DISCLOSURE

Curable compositions are often used in the additive manufacturing of polymeric materials, such as those used for the fabrication of medical devices. There is a need for new polymeric materials, as well as methods for producing the same, that provide desirable mechanical properties for use in various device applications, such as for medical devices.

### SUMMARY OF THE DISCLOSURE

The present disclosure provides a compound of Formula (I):

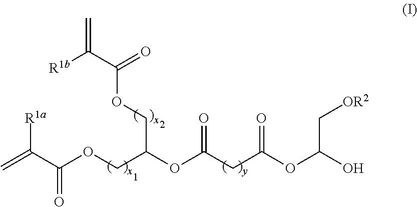

wherein:
R^{1a} and R^{1b} are, each independently, H, halogen, or C₁-C₆ alkyl; R² is H, C₁-C₂₀ alkyl, —C(═O)C₁-C₂₀ alkyl, C₁-C₆ haloalkyl, acrylate, silyl ether, or a moiety comprising a photoinitiator; and x₁, x₂, and y are, each independently, 1, 2 or 3.

Embodiments of the present disclosure provide a compound of Formula (VI):

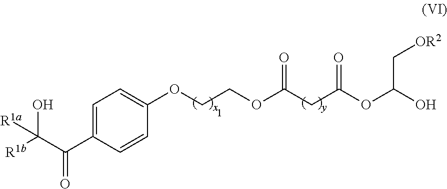

wherein:
R^{1a} and R^{1b} are, each independently, H, halogen, or C₁-C₆ alkyl; R² is H, C₁-C₂₀ alkyl, —C(═O)C₁-C₂₀ alkyl, C₁-C₆ haloalkyl, acrylate, silyl ether, or a moiety comprising a photoinitiator; and x₁ and y are, each independently, 1, 2 or 3

Embodiments of the present disclosure provide a composition comprising a compound of any one of claims 1-11 and an oligomer comprising a glass transition temperature (Ts) of at most −10° C.

Additional embodiments of the present disclosure provide method for synthesizing an oligomer compound.

Another embodiment of the present disclosure provides a method of synthesizing a compound of Formula (I). The method comprises reacting a compound of Formula (Ia) with an epoxide of Formula (Ib):

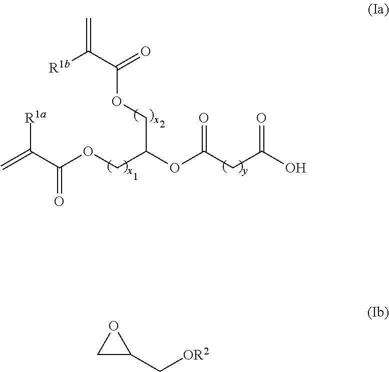

wherein:
R^{1a} and R^{1b} are, each independently, H, halogen, or C₁-C₆ alkyl; R² is H, C₁-C₂₀ alkyl, —C(═O)C₁-C₂₀ alkyl, C₁-C₆ haloalkyl, acrylate, silyl ether, or a moiety comprising a photoinitiator; and x₁, x₂, and y are each independently 1, 2, or 3.

Another embodiment of the present disclosure provides a method of synthesizing a compound of Formula (VI). The method comprises reacting a compound of Formula (VIa) with an epoxide of Formula (Ib):

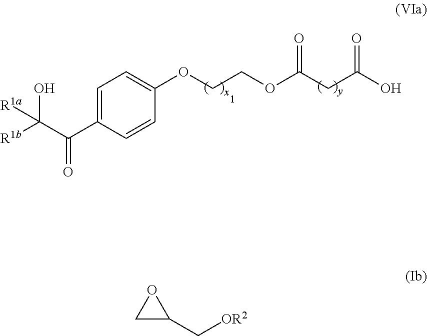

wherein:
R^{1a} and R^{1b} are, each independently, H, halogen, or C₁-C₆ alkyl; R² is H, C₁-C₂₀ alkyl, —C(═O)C₁-C₂₀ alkyl, C₁-C₆ haloalkyl, acrylate, silyl ether, or a moiety comprising a photoinitiator; and x₁, and y are each independently 1, 2, or 3.

Further, embodiments of the present disclosure provide a polymerizable composition. The polymerizable composition comprises the oligomer of the present disclosure, a reactive diluent, and an initiator. embodiments of the present disclosure also provide a polymer made from the polymerizable composition.

One embodiment provides an orthodontic appliance comprising a multi-functional polymerizable compounds of the disclosure. In some embodiments, the orthodontic appliance is a dental appliance. In certain embodiments, the orthodontic appliance is a dental aligner, a dental expander, or a dental spacer.

Additional embodiments provide a method for preparing an article by an additive manufacturing process, comprising:
providing a first polymerizable composition;
exposing the first polymerizable composition to radiation;
polymerizing the first polymerizable composition layer-by-layer based on a predefined design, thereby polymerizing the first polymerizable composition to form a polymer;
fabricating the article with the polymer; and
contacting the article with a second polymerizable composition thereby forming a polymer coating.

Other embodiments provide a method of repositioning a patient's teeth, comprising:
generating a treatment plan for the patient, the plan comprising a plurality of intermediate tooth arrangements for moving teeth along a treatment path from an initial tooth arrangement toward a final tooth arrangement;
producing an orthodontic appliance or an orthodontic appliance comprising the multi-functional polymerizable compounds of the disclosure; and
moving on-track, with the orthodontic appliance, at least one of the patient's teeth toward an intermediate tooth arrangement or the final tooth arrangement.

### DESCRIPTION OF THE FIGURES

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1A illustrates a tooth repositioning appliance, in accordance with embodiments.

FIG. 1B illustrates a tooth repositioning system, in accordance with embodiments.

FIG. 1C illustrates a method of orthodontic treatment using a plurality of appliances, in accordance with embodiments.

FIG. 2 is a partially schematic illustration of a system for additive manufacturing configured in accordance with embodiments of the present technology.

FIG. 3 illustrates a method for digitally planning an orthodontic treatment, in accordance with embodiments.

FIG. 4 shows generating and administering treatment according to an embodiment of the present disclosure.

FIG. 5 shows a schematic configuration of a high temperature additive manufacturing device by using a 3D printing process.

FIG. 6 illustrates a method for designing an orthodontic appliance, in accordance with embodiments.

### DETAILED DESCRIPTION

The present disclosure provides muti-functional polymerizable compounds and chemical synthesis of the same. The present disclosure provides useful compounds and compositions as well as methods of using (e.g., for producing polymeric material) and producing the same. The polymerizable compositions described herein can address an unmet need to produce oligomers with advantageous properties (e.g., increased toughness, smoothness, stain resistance) useful for various device applications.

All terms, chemical names, expressions, and designations have their usual meanings which are well-known to those skilled in the art. As used herein, the terms “to comprise” and “comprising” are to be understood as non-limiting, i.e., other components than those explicitly named may be included. The term “consisting” or “consisting of” means that only components that are explicitly described are included. The term “consisting essentially of” limits the scope to specified materials, elements, steps, embodiments, aspects, and limitations except for those that do not materially affect basic and novel characteristics. For each embodiment of this disclosure, it is understood that any specified materials, elements, steps, embodiments, aspects, and limitations may be included with any of the phrases.

Number ranges are to be understood as inclusive, i.e., including the indicated lower and upper limits (e.g., the phrase “an integer ranging from 1-3” includes the integers 1, 2, and 3). Furthermore, the term “about,” as used herein, and unless clearly indicated otherwise, refers to and encompasses plus or minus 10% of the indicated numerical value(s). For example, “about 10%” may indicate a range of 9% to 11%, and “about 1” may include the range 0.9-1.1.

As used herein, the terms “polymer,” “polymeric material,” or an equivalent refers to a molecule composed of repeating structural units connected by covalent chemical bonds and characterized by a substantial number of repeating units (e.g., equal to or greater than 10 repeating units; in some embodiments, repeating units are equal to or greater than 100, 200, 250, 300, 350, 400, 450, or 500 repeating units) and a molecular weight greater than or equal to 5,000 Daltons (Da) or 5 kDa; for example, in some embodiments, a polymeric material has a molecular weight greater than or equal to 10 kDa, 15 kDa, 20 kDa, 30 kDa, 40 kDa, 50 kDa, or 100 kDa. Polymers of the present disclosure are the polymerization product of a diradical photoinitiator of this disclosure and (optionally) one or more monomer components. The term polymer includes homopolymers, i.e., polymers consisting essentially of a single repeating monomer species. The term polymer also includes copolymers which are formed when two or more different types (or species) of monomers are linked in the same polymer. Copolymers may comprise two or more different monomer species, and include random, block, alternating, segmented, grafted, tapered and other copolymers.

As used herein, the term “reactive diluent” generally refers to a substance which reduces the viscosity of another substance, such as a monomer or curable resin or polymerizable composition. A reactive diluent may become part of another substance, such as a polymer obtained by a polymerization process. In some examples, a reactive diluent is a curable monomer which, when mixed with a curable resin or polymerizable composition, reduces the viscosity of the resultant formulation, and is incorporated into the polymer that results from polymerization of the formulation.

The average molecular weight (M) is the average number of repeating units n times the molecular weight or molar mass (Mi) of the repeating unit. The number-average molecular weight (M) is the arithmetic mean, representing the total weight of the molecules present divided by the total number of molecules.

The term “biocompatible,” as used herein, refers to a material that does not elicit an immunological rejection or detrimental effect, referred herein as an adverse immune response when it is disposed within an in vivo biological environment. For example, in embodiments a biological marker indicative of an immune response changes less than 10%, or less than 20%, or less than 25%, or less than 40%, or less than 50% from a baseline value when a human or animal is exposed to or in contact with the biocompatible material. Alternatively, immune response may be determined histologically, wherein localized immune response is assessed by visually assessing markers, including immune cells or markers that are involved in the immune response pathway, in and adjacent to the material. In an aspect, a biocompatible material or device does not observably change immune response as determined histologically. In some embodiments, the disclosure provides biocompatible devices configured for long-term use, such as on the order of weeks to months, without invoking an adverse immune response. Biological effects may be initially evaluated by measurement of cytotoxicity, sensitization, irritation and intracutaneous reactivity, acute systemic toxicity, pyrogenicity, subacute/subchronic toxicity and/or implantation. Biological tests for supplemental evaluation include testing for chronic toxicity.

“Bioinert” refers to a material that does not elicit an immune response from a human or animal when it is disposed within an in vivo biological environment. For example, a biological marker indicative of an immune response remains substantially constant (plus or minus 5% of a baseline value) when a human or animal is exposed to or in contact with the bioinert material. In some embodiments, the disclosure provides bioinert devices.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and sub-combinations possible of the group are intended to be individually included in the disclosure. When a compound is described herein such that a particular isomer, enantiomer, or diastereomer of the compound is not specified, for example, in a formula or in a chemical name, that description is intended to include each isomer and enantiomer of the compound described individually or in any combination. Additionally, unless otherwise specified, all isotopic variants of compounds disclosed herein are intended to be encompassed by the disclosure. Specific names of compounds are intended to be exemplary, as it is known that one of ordinary skill in the art can name the same compounds differently.

It is noted that as used herein and in the appended claims, the singular forms “a”, “an”, and “the” include plural reference unless the context clearly dictates otherwise. Thus, for example, reference to “a monomer” includes a plurality of such monomers and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

As used herein, the term “group” or “moiety” may refer to a reactive functional group of a chemical compound. Groups of the present compounds refer to an atom or a collection of atoms that are a part of the compound. Groups of the present disclosure may be attached to other atoms of the compound via one or more covalent bonds. Groups may also be characterized with respect to their valence state. The present disclosure includes groups characterized as monovalent, divalent, trivalent, etc. valence states.

As used herein, the term “substituted” refers to a compound (e.g., an alkyl chain) wherein a hydrogen is replaced by another reactive functional group or atom, as described herein.

As used herein, a [symbol] symbol in, e.g.,

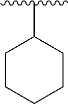 [SMILES: *C1CCCCC1]

indicates that the given moiety, the cyclohexyl moiety in this example, is attached to a molecule (e.g., via a polymer backbone) via the bond that is “capped” with the wavy line.

“Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching but does not include carbon atoms belonging to any substituents. Unless stated otherwise specifically in the specification, an alkyl group is optionally substituted.

“Alkenyl” refers to an unsaturated, straight or branched hydrocarbon chain radical consisting solely of carbon and hydrogen atoms, which contains one or more carbon-carbon double bonds, having from two to twelve carbon atoms (C₂-C₁₂ alkenyl), two to eight carbon atoms (C₂—C₈ alkenyl) or two to six carbon atoms (C₂-C₆ alkenyl), or any value within these ranges, and which is attached to the rest of the molecule by a single bond, e.g., ethenyl, prop-1-enyl, but-1-enyl, pent-1-enyl, penta-1,4-dienyl, and the like. The number of carbons referred to relates to the carbon backbone and carbon branching but does not include carbon atoms belonging to any substituents. Unless stated otherwise specifically in the specification, an alkenyl group is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₅ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

Aryl groups include groups having one or more 5-, 6-, 7- or 8-membered aromatic rings, including heterocyclic aromatic rings. The term heteroaryl specifically refers to aryl groups having at least one 5-, 6-, 7- or 8-member heterocyclic aromatic ring. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those that are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semi-halogenated aryl groups, such as aryl groups having one or more hydrogens replaced with one or more fluorine atoms, chlorine atoms, bromine atoms and/or iodine atoms. Substituted aryl groups include fully fluorinated or semi-fluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein provided in a covalently bonded configuration in the compounds of the disclosure at any suitable point of attachment. In some embodiments, aryl groups contain between 5 and 30 carbon atoms. In some embodiments, aryl groups contain one aromatic or heteroaromatic six-member ring and one or more additional five- or six-member aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents.

Arylalkyl groups are alkyl groups substituted with one or more aryl groups wherein the alkyl groups optionally carry additional substituents, and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents, and the aryl groups are optionally substituted. Specific alkylaryl groups are alkyl-substituted phenyl groups such as methylphenyl. Substituted arylalkyl groups include fully halogenated or semi-halogenated arylalkyl groups, such as arylalkyl groups having one or more alkyl and/or aryl groups having one or more hydrogens replaced with one or more fluorine atoms, chlorine atoms, bromine atoms and/or iodine atoms.

As used herein, the terms “halo” and “halogen” can be used interchangeably and refer to a halogen group such as a fluoro (—F), chloro (—Cl), bromo (—Br) or iodo (—I)

The term “heterocyclic” or “heterocyclyl” refers to ring structures containing at least one other kind of atom, in addition to carbon, in the ring. Examples of such heteroatoms include nitrogen, oxygen, and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and reactive functional groups, for example, provided as substituents.

The term “heteroalkyl,” as used herein, generally refers to an alkyl, alkenyl, or alkynyl group as defined herein, wherein at least one carbon atom of the alkyl group is replaced with a heteroatom. In some instances, heteroalkyl groups may contain from 1 to 18 non-hydrogen atoms (carbon and heteroatoms) in the chain, or from 1 to 12 non-hydrogen atoms, or from 1 to 6 non-hydrogen atoms, or from 1 to 4 non-hydrogen atoms. Heteroalkyl groups may be straight or branched, and saturated or unsaturated. Unsaturated heteroalkyl groups have one or more double bonds and/or one or more triple bonds. Heteroalkyl groups may be unsubstituted or substituted. Exemplary heteroalkyl groups include, but are not limited to, alkoxyalkyl (e.g., methoxymethyl), and aminoalkyl (e.g., alkylaminoalkyl and dialkylaminoalkyl). Heteroalkyl groups may be optionally substituted with one or more substituents.

The term “carbonyl” or “oxo,” as used herein, for example in the context of C₁₋₆ carbonyl substituents, generally refers to a carbon chain of given length (e.g, C₁₋₆), wherein each of the carbon atom of a given carbon chain can form the carbonyl bond, as long as it is chemically feasible in terms of the valence state of that carbon atom. Thus, in some instances, the “C₁₋₆ carbonyl” substituent refers to a carbon chain of between 1 and 6 carbon atoms, and either the terminal carbon contains the carbonyl functionality, or an inner carbon contains the carbonyl functionality, in which case the substituent could be described as a ketone. The term “carboxy”, as used herein, for example in the context of C₁₋₆ carboxyl substituents, generally refers to a carbon chain of given length (e.g., C₁₋₆), wherein a terminal carbon contains the carboxy functionality, unless otherwise defined herein.

As to any of the groups described herein that contain one or more substituents, it is understood that such groups do not contain any substitution or substitution patterns which are sterically impractical and/or synthetically non-feasible. In addition, the compounds of this disclosure include all stereochemical isomers arising from the substitution of these compounds.

Unless otherwise defined herein, optional substituents for any alkyl, alkenyl, and aryl group includes substitution with one or more of the following substituents, among others:
halogen, including fluorine, chlorine, bromine or iodine;
pseudohalides, including —CN, —OCN (cyanate), —NCO (isocyanate), —SCN
(thiocyanate) and —NCS (isothiocyanate);

—COOR, where R is a hydrogen or an alkyl group or an aryl group and more specifically where R is a methyl, ethyl, propyl, butyl, or phenyl group all of which groups are optionally substituted;

—COR, where R is a hydrogen or an alkyl group or an aryl group and more specifically where R is a methyl, ethyl, propyl, butyl, or phenyl group all of which groups are optionally substituted;

—CON(R)₂, where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂, where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂, where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring that can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR, where R is an alkyl group or an aryl group and more specifically where R is a methyl, ethyl, propyl, butyl, or phenyl group, all of which are optionally substituted;

—OCOOR, where R is an alkyl group or an aryl group;

—SO₂N(R)₂, where each R, independently of each other R, is a hydrogen, or an alkyl group, or an aryl group all of which are optionally substituted and wherein R and R can form a ring that can contain one or more double bonds and can contain one or more additional carbon atoms; and

—OR, where R is H, an alkyl group, an aryl group, or an acyl group all of which are optionally substituted. In a particular example R can be an acyl yielding —OCOR″, wherein R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

“Heteroalkylene” refers to an alkylene group, as defined above, comprising at least one heteroatom (e.g., Si, N, O, P or S) within the alkylene chain or at a terminus of the alkylene chain. In some embodiments, the heteroatom is within the alkylene chain.

“Cycloalkylene” refers to a stable non-aromatic monocyclic or polycyclic carbocyclic ring with one or more C═C bond, which may include fused or bridged ring systems, having from three to fifteen carbon atoms, preferably having from three to ten carbon atoms, and which is unsaturated and attached to the rest of the molecule by a single bond.

Heterocyclylene refers to a stable non-aromatic monocyclic or polycyclic heterocarbocyclic ring with one or more C═C bond, which may include fused or bridged ring systems, having from three to fifteen carbon atoms, preferably having from three to ten carbon atoms, and which is unsaturated and attached to the rest of the molecule by a single bond.

The term “mouthguard” refers to a protective device for a user's mouth that covers the teeth and gums to prevent and/or reduce injury to the teeth, lips, and gums. The disclosed mouthguards can be used for sports, to treat teeth griding, or as a part of certain dental procedures. The mouthguards can be also described as a mouth protector, sportsguard, mouth piece, gumshield, gumguard, nightguard, occlusal splint, bite splint, bite plane, bite positioners, or jaw positioners.

The disclosed mouthguards can be used as a carrier for intraoral sensors for tracking patient data such as but not limited to temperature, pH-value, tracking of chemical indicators, bite forces, grinding forces, and acceleration during sports activities or impact events.

As to any of the above groups that contain one or more substituents, it is understood that such groups do not contain any substitution or substitution patterns which are sterically impractical and/or synthetically non-feasible. In addition, as further described herein, the compounds of this disclosure can include all stereochemical isomers (and racemic mixtures) arising from the substitution of these compounds.

### Method for Preparing Muti-Functional Polymerizable Compounds

The present disclosure provides a method for preparing multi-functional polymerizable compounds. In some embodiments, the multi-functional polymerizable compounds is also used as capping agents for preparing urethane-based polymers. The present disclosure also provides multi-functional polymerizable compounds comprising a plurality of reactive functional groups (e.g., two or three reactive functional groups). Such a multi-functional polymerizable compound can be a monomer or an oligomer. The monomer or oligomer can be linear or branched.

The multi-functional polymerizable compounds are obtained via a two-step synthetic route with high yield and high purity without a column chromatography purification step. A synthesis of the multi-functional polymerizable compounds is illustrated below:

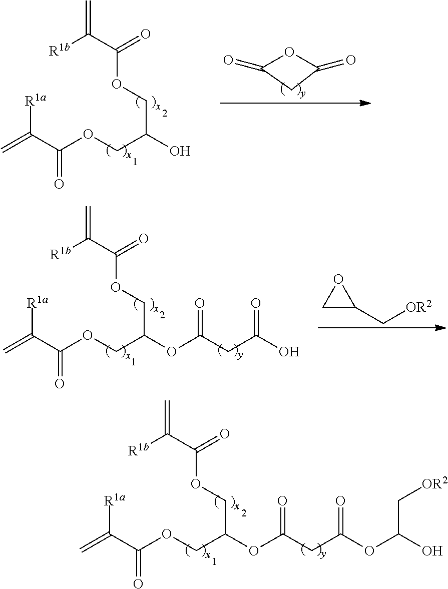

A compound having the structure of Formula (I) or a salt thereof is described.

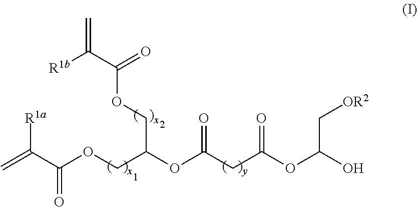

wherein:
R^{1a} and R^{1b} are, each independently, H, halogen, or C₁-C₆ alkyl; R² is H, C₁-C₂₀ alkyl, —C(═O)C₁-C₂₀ alkyl, C₁-C₆ haloalkyl, acrylate, silyl ether, or a moiety comprising a photoinitiator; and x₁, x₂, and y are, each independently, 1, 2 or 3.

In some embodiments, the multi-functional polymerizable compounds are obtained via a two-step synthetic route with high yield and high purity without a column chromatography purification step. A synthesis of the multi-functional polymerizable compounds is illustrated below:

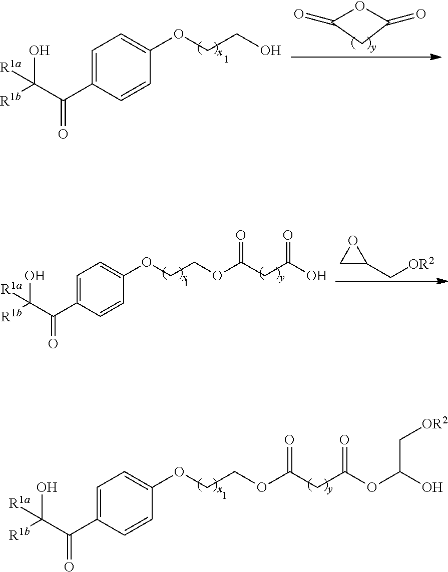

In some embodiments, a compound having the structure of Formula (VI) or a salt thereof is described.

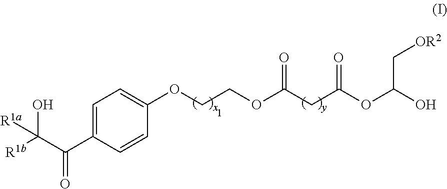

wherein:
R^{1a} and R^{1b} are, each independently, H, halogen, or C₁-C₆ alkyl; R² is H, C₁-C₂₀ alkyl, —C(═O)C₁-C₂₀ alkyl, C₁-C₆ haloalkyl, acrylate, silyl ether, or a moiety comprising a photoinitiator; and x₁ and y are, each independently, 1, 2 or 3.

In one embodiment, R^{1a} and R^{1b} are, each independently, H, halogen, or C₁-C₆ alkyl. In some embodiments, R^{1a} and R^{1b} are H. In some embodiments, R^{1a} and R^{1b} are halogen. In some embodiments, R^{1a} and R^{1b} are C₁-C₆ alkyl. In some embodiments, R^{1a} and R^{1b} are, each independently, H or C₁-C₃ alkyl. In some embodiments, R^{1a} and R^{1b} are, each independently, H or C₁ alkyl. In some specific embodiments, R^{1a} and R^{1b} are, each independently, H or —CH₃. In some other specific embodiments, R^{1a} or R^{1b} is H.

In one embodiment, R² is H, C₁-C₂₀ alkyl, —C(═O)C₁-C₂₀ alkyl, C₁-C₆ haloalkyl, acrylate, silyl ether, or a moiety comprising a photoinitiator. In some embodiments, R² is H. In some embodiments, R² is C₁-C₂₀ alkyl. In some embodiments, R² is C₁-C₁₅ alkyl. In some embodiments, R² is C₁-C₁₆ alkyl. In some embodiments, R² is C₆-C₂₀ alkyl. In some embodiments, R² is C₆-C₁₈ alkyl. In some specific embodiments, R² is C₆-C₁₆ alkyl. In some specific embodiments, R² is C₆-C₁₆ alkyl. In some embodiments, R² is C₁₄ alkyl. In some specific embodiments, R² is C₆-C₁₆ alkyl. In some embodiments, R² is C₁₅ alkyl. In some embodiments, R² is C₁₆ alkyl. In some other specific embodiments, R² is C₆-C₁₆ alkyl. In some embodiments, R² is C₁₇ alkyl. In some other specific embodiments, R² is C₆-C₁₆ alkyl. In some embodiments, R² is C₁₈ alkyl. In some other specific embodiments, R² is C₆-C₁₆ alkyl. In some embodiments, R² is C₆ alkyl. In some other specific embodiments, R² is C₆-C₁₆ alkyl. In some embodiments, R² is C₇ alkyl. In some other specific embodiments, R² is C₆-C₁₆ alkyl. In some embodiments, R² is C₈ alkyl. In some other specific embodiments, R² is C₆-C₁₆ alkyl. In some embodiments, R² is C₉ alkyl. In some other specific embodiments, R² is C₆-C₁₆ alkyl. In some embodiments, R² is C₁₀ alkyl.

In some embodiments, R² is —C(═O)C₁-C₂₀ alkyl. In some embodiments, R² is —C(═O)C₁-C₁₈ alkyl. In some embodiments, R² is —C(═O)C₁-C₁₆ alkyl. In some embodiments, R² is —C(═O)C₁₂-C₂₀ alkyl. In some embodiments, R² is —C(═O)C₁₂-C₁₅ alkyl. In some embodiments, R² is —C(═O)C₁₂-C₁₆ alkyl. In some specific embodiments, R² is —C(═O)C₁₄ alkyl. In some specific embodiments, R² is —C(═O)C₁₅ alkyl. In some specific embodiments, R² is —C(═O)C₁₆ alkyl.

In some embodiments, R² is C₁-C₆ haloalkyl. In some embodiments, R² is C₂-C₄ haloalkyl. In some embodiments, R² is C₂ haloalkyl. In some embodiments, R² is C₃ haloalkyl. In some embodiments, R² is C₄ haloalkyl. In some embodiments, R² is C₄ fluoroalkyl. In some embodiments, R² is C₄ di, tri, or tetra-fluoroalkyl. In some embodiments, R² is C₄ tetra-fluoroalkyl. In some specific embodiments, R² is 1,1,2,2-tetrafluoropropyl.

In some embodiments, R² is acrylate. The acrylate of R² has the following structure:

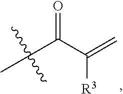

wherein R³ is H, halogen, or C₁-C₆ alkyl. In some embodiments, R³ is H, halogen, or C₁-C₃ alkyl. In some specific embodiments, R³ is H, halogen, or C₁ alkyl. In some embodiments, R³ is H. In some embodiments, R³ is halogen. In some embodiments, R³ is C₁-C₃ alkyl. In some embodiments, R³ is C₁ alkyl. In some embodiments, R³ is a methyl (—CH₃). In some embodiments, R³ is C₂ alkyl. In some embodiments, R³ is C₃ alkyl.

In some embodiments, R² is silyl ether. The silyl ether of R² has the following structure:

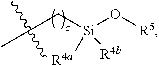

wherein R^{4a} and R^{4b} are, each independently H or C₁-C₆ alkyl; R⁵ is H, C₁-C₆ alkyl, or silyl; and z is an integer from 1 to 5. In some embodiments, R^{4a} and R^{4b} are, each independently H or C₁-C₄ alkyl. In some embodiments, R^{4a} and R^{4b} are, each independently H or C₁-C₃ alkyl. In some specific embodiments, R^{4a} and R^{4b} are, each independently H or C₁ alkyl. For example, R^{4a} and R^{4b} are, each independently H or a methyl (—CH₃).

In some embodiments, R⁵ is H, C₁-C₄ alkyl, or silyl. In some specific embodiments, R⁵ is silyl. In some specific embodiments, the silyl of R⁵ is dialkyl silyl. In some specific embodiments, the silyl of R⁵ is dimethyl silyl.

In some embodiments, z is an integer from 1 to 4. In some embodiments, z is an integer of 1. In some embodiments, z is an integer of 2. In some embodiments, z is an integer of 3. In some embodiments, z is an integer of 4. In some embodiments, z is an integer of 5. In some embodiments, R² is acrylate or silyl ether.

In some embodiments, R² is a moiety comprising a photoinitiator. The photoinitiator refers to a molecule that creates reactive species such as free radicals, cations, or anions when exposed to radiation. In some embodiments, R² is a moiety comprising a chemical structure with a photoinitiator capability including azobisisobutyronitrile, benzyl peroxide, 2,2-dimethoxy-2-phenylacetophenone, camphorquinone, BAPO Bis(2,4,6-trimethylbenzoyl)-phenylphosphineoxide, or the like. For instance, in some embodiments, R² is a moiety including a part of the following photoinitiator structures: azobisisobutyronitrile, benzyl peroxide, 2,2-dimethoxy-2-phenylacetophenone, camphorquinone, and BAPO Bis(2,4,6-trimethylbenzoyl)-phenylphosphineoxide. In some specific embodiments, the photoinitiator or R² has the following structure:

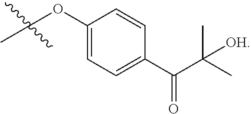 [SMILES: *Oc1ccc(C(=O)C(C)(C)O)cc1]

In some embodiments, R² has one of the following structures:

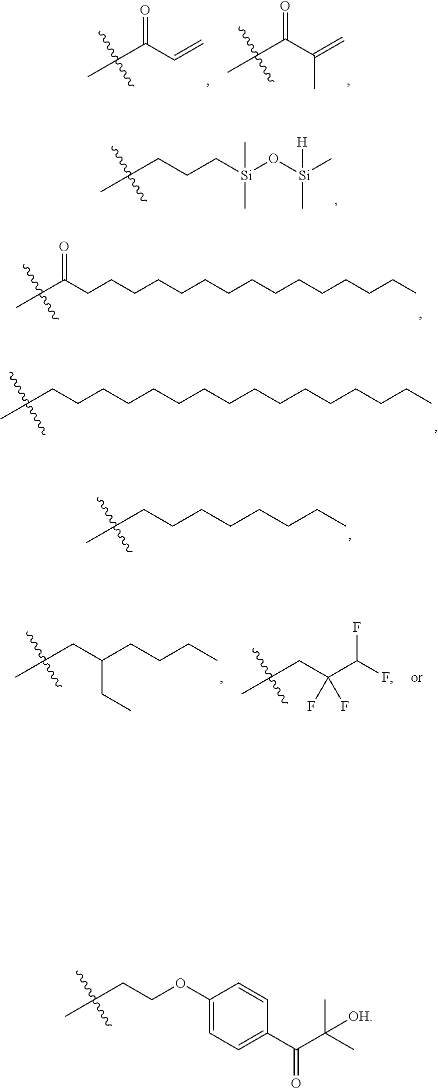

In some embodiments, R² is

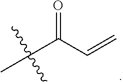 [SMILES: *C(=O)C=C]

In some embodiments, R² is

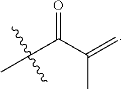 [SMILES: *C(=O)C(=C)C]

In some embodiments, R² is

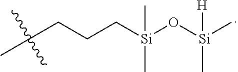 [SMILES: *CCC[Si](C)(C)O[SiH](C)C]

In some embodiments, R² is

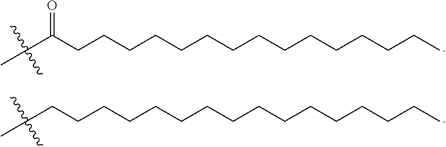 [SMILES: *C(=O)CCCCCCCCCCCCCCC ; *CCCCCCCCCCCCCCCC]

In some embodiments, R² is
In some embodiments, R² is

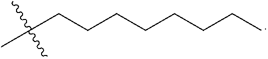 [SMILES: *CCCCCCCC]

In some embodiments, R² is

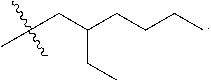 [SMILES: *CC(CC)CCCC]

In some embodiments, R² is

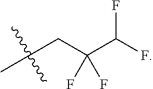 [SMILES: *CC(F)(F)C(F)F]

In some embodiments, R² is O

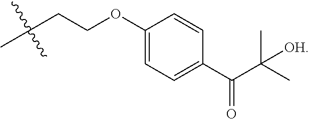 [SMILES: *CCOc1ccc(C(=O)C(C)(C)O)cc1]

In some embodiments, x₁, x₂, and y are, each independently, 1, 2 or 3. In some embodiments, x₁, x₂, and y are, each independently, 1 or 2. In some specific embodiments, x₁ and x₂ are 1. In some embodiments, y is 2.

The present disclosure provides a facile two-step synthetic route to tri(meth)acrylate compounds with quantitative conversion at each step, requiring only an aqueous work-up after each step for purification. These trifunctional compounds have not been utilized for oligomer/polymer end-functionalization previously. Here, the tri(meth)acrylate compounds are used for end-capping diisocyanate prepolymers to result in hexafunctional polyurethanes for incorporation into UV-curable formulations. UV-curable formulations containing such a multifunctional oligomer component can be photopolymerized to yield high-modulus materials due to the resulting high crosslinking density. The tri(meth)acrylate compounds having the Formula (I) can also be used as reactive diluents in photocurable resin formulations. When the multi-functional polymerizable compounds (i.e., a capping agent) are used for end-capping a low-Tg oligomer backbone resulting in a multifunctional oligomer with multiple (meth)acrylate end groups (e.g., two or more), it can be formulated with a high-Tg reactive diluent whereby the formulation composition can be tuned by increasing the amount of the oligomer component relative to the reactive diluent component to still maintain a relatively high flexural modulus while imparting toughness to the material. Such viscous formulations are suitable for high temperature lithography-based photopolymerization processes.

Embodiments of the present disclosure provide advantages for improving stain resistance by introducing hydrophobic groups, brush type polymers for impact, and introduction of silanes, siloxanes, and other hydrophobic groups to the oligomer for potential adhesion promotion and coating purposes.

Muti-functional polymerizable compounds described herein can, in a polymerized form, have a glass transition temperature (Tg) from about −100° C. to about 200° C., from about −50° C. to about 200° C., from about −0° C. to about 200° C., from about −0° C. to about 150° C., or from about 0° C. to about 100° C.

TABLE 1
Exemplary Compounds of the Disclosure

| Compound ID | Structure |
|---|---|
| I-1 | 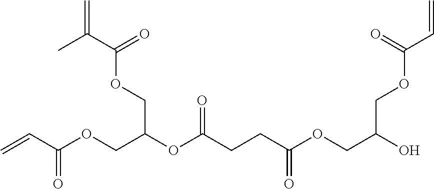 [SMILES: C=CC(=O)OCC(O)COC(=O)CCC(=O)OC(COC(=O)C=C)COC(=O)C(=C)C] |
| I-2 | 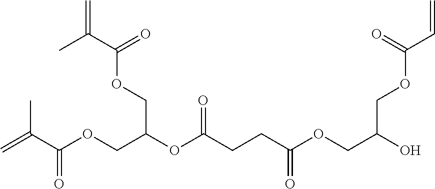 [SMILES: C=CC(=O)OCC(O)COC(=O)CCC(=O)OC(COC(=O)C(=C)C)COC(=O)C(=C)C] |
| I-3 | 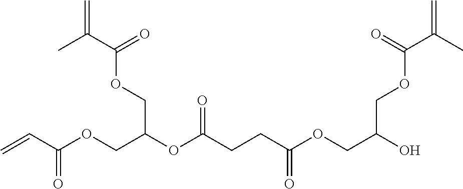 [SMILES: C=CC(=O)OCC(COC(=O)C(=C)C)OC(=O)CCC(=O)OCC(O)COC(=O)C(=C)C] |
| I-4 | 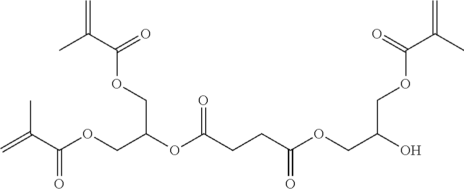 [SMILES: C=C(C)C(=O)OCC(O)COC(=O)CCC(=O)OC(COC(=O)C(=C)C)COC(=O)C(=C)C] |
| I-5 | 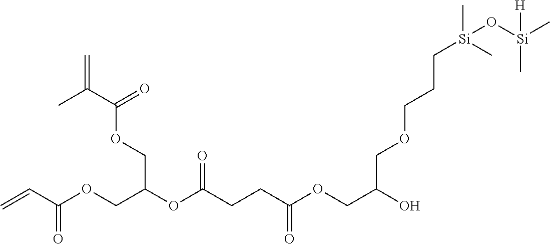 [SMILES: C=CC(=O)OCC(COC(=O)C(=C)C)OC(=O)CCC(=O)OCC(O)COCCC[Si](C)(C)O[SiH](C)C] |
| I-6 | 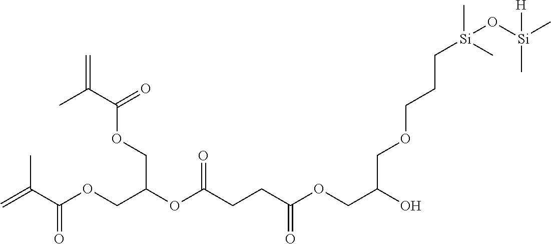 [SMILES: C=C(C)C(=O)OCC(COC(=O)C(=C)C)OC(=O)CCC(=O)OCC(O)COCCC[Si](C)(C)O[SiH](C)C] |
| I-7 | 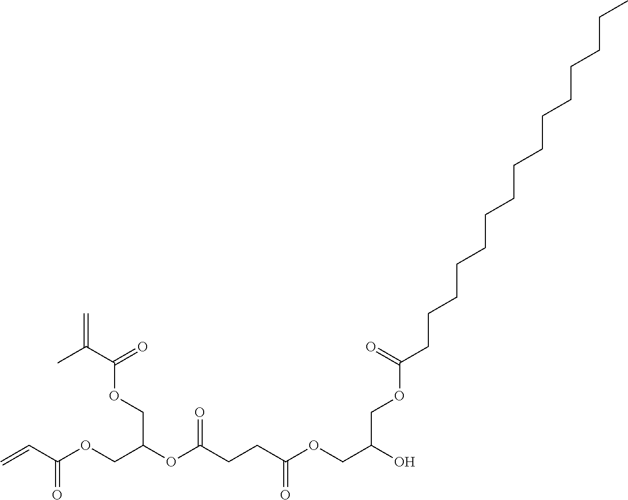 [SMILES: C=CC(=O)OCC(COC(=O)C(=C)C)OC(=O)CCC(=O)OCC(O)COC(=O)CCCCCCCCCCCCCCC] |
| I-8 | 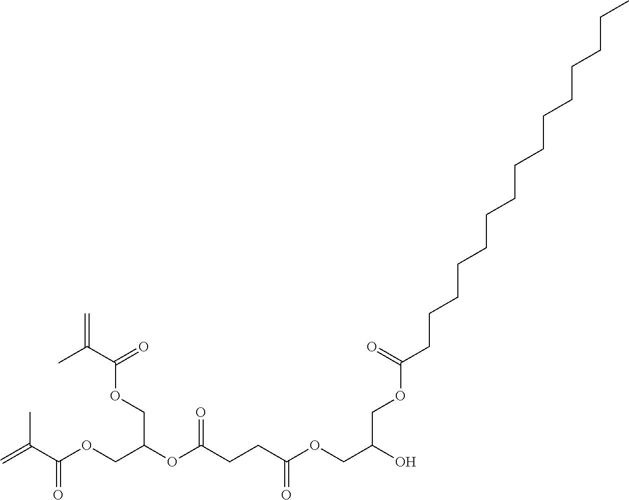 [SMILES: C=C(C)C(=O)OCC(COC(=O)C(=C)C)OC(=O)CCC(=O)OCC(O)COC(=O)CCCCCCCCCCCCCCC] |
| I-9 | 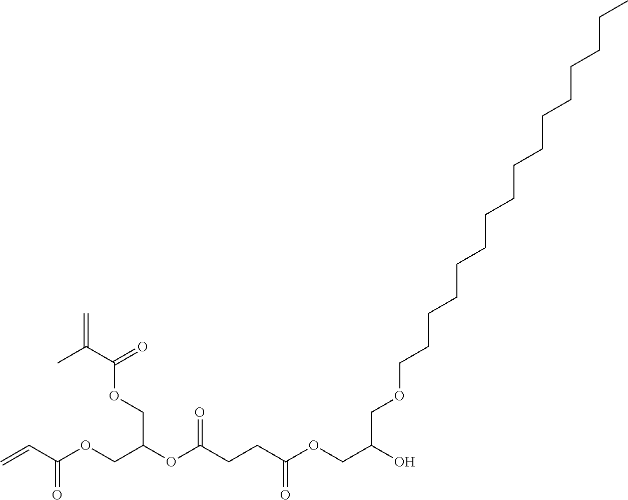 [SMILES: C=CC(=O)OCC(COC(=O)C(=C)C)OC(=O)CCC(=O)OCC(O)COCCCCCCCCCCCCCCCC] |
| I-10 | 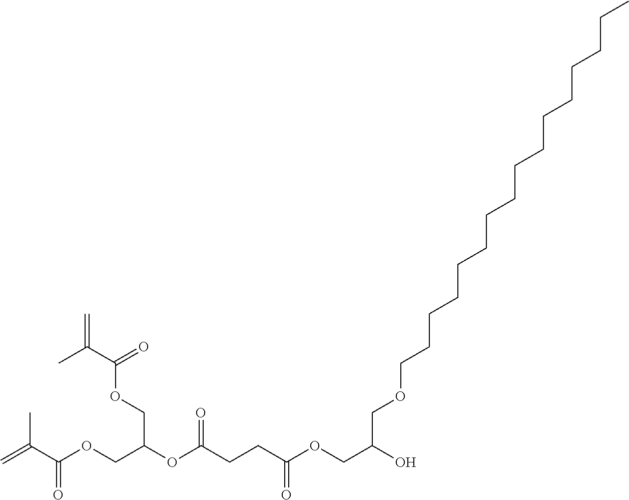 [SMILES: C=C(C)C(=O)OCC(COC(=O)C(=C)C)OC(=O)CCC(=O)OCC(O)COCCCCCCCCCCCCCCCC] |
| I-11 | 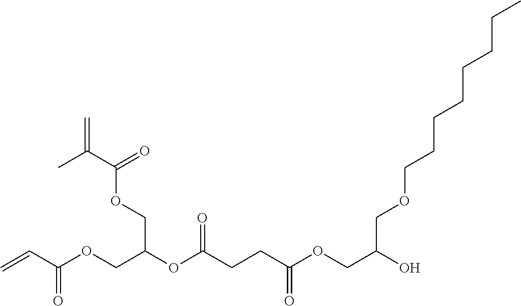 [SMILES: C=CC(=O)OCC(COC(=O)C(=C)C)OC(=O)CCC(=O)OCC(O)COCCCCCCCC] |
| I-12 | 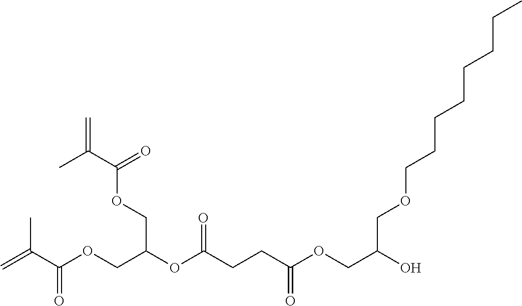 [SMILES: C=C(C)C(=O)OCC(COC(=O)C(=C)C)OC(=O)CCC(=O)OCC(O)COCCCCCCCC] |
| I-13 | 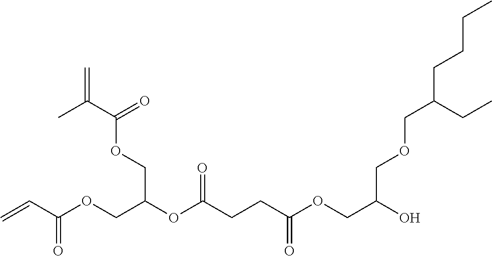 [SMILES: C=CC(=O)OCC(COC(=O)C(=C)C)OC(=O)CCC(=O)OCC(O)COCC(CC)CCCC] |
| I-14 | 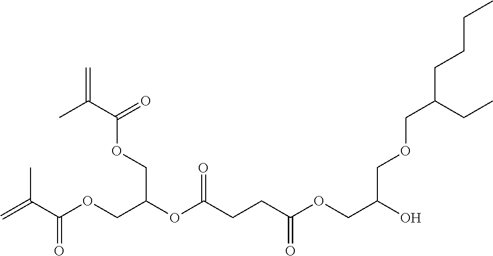 [SMILES: C=C(C)C(=O)OCC(COC(=O)C(=C)C)OC(=O)CCC(=O)OCC(O)COCC(CC)CCCC] |
| I-15 | 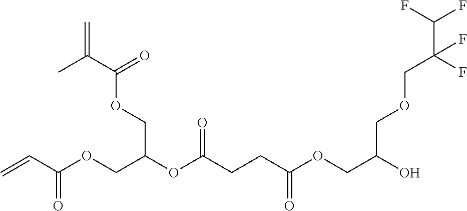 [SMILES: C=CC(=O)OCC(COC(=O)C(=C)C)OC(=O)CCC(=O)OCC(O)COCC(F)(F)C(F)F] |
| I-16 | 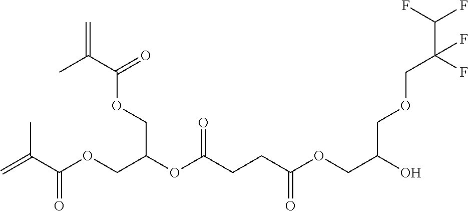 [SMILES: C=C(C)C(=O)OCC(COC(=O)C(=C)C)OC(=O)CCC(=O)OCC(O)COCC(F)(F)C(F)F] |
| I-17 | 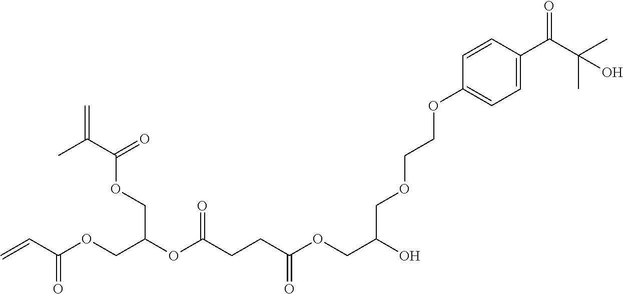 [SMILES: C=CC(=O)OCC(COC(=O)C(=C)C)OC(=O)CCC(=O)OCC(O)COCCOc1ccc(C(=O)C(C)(C)O)cc1] |
| I-18 | 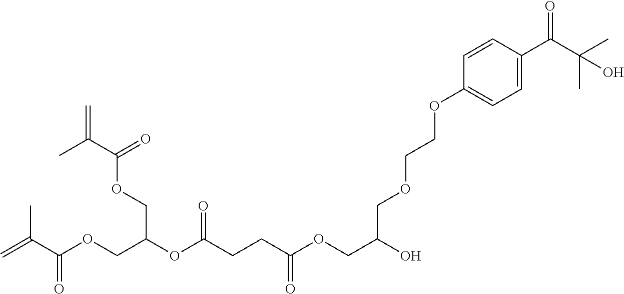 [SMILES: C=C(C)C(=O)OCC(COC(=O)C(=C)C)OC(=O)CCC(=O)OCC(O)COCCOc1ccc(C(=O)C(C)(C)O)cc1] |
| VI-1 | 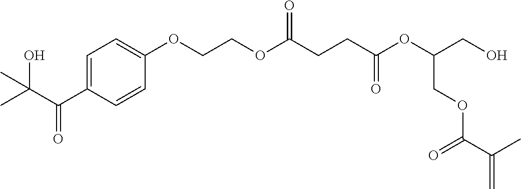 [SMILES: C=C(C)C(=O)OCC(CO)OC(=O)CCC(=O)OCCOc1ccc(C(=O)C(C)(C)O)cc1] |

### Method for Preparing an Oligomer Using Muti-Functional Polymerizable Compounds

Embodiments of the present disclosure provide a method for preparing an oligomer using multi-functional polymerizable compounds and a synthesis of the oligomer is illustrated below:

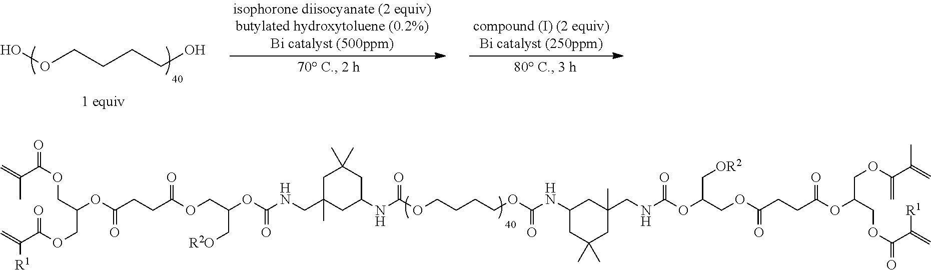

In one embodiment, a method for synthesizing the compound having the structure of Formula (I) is described. The method comprises reacting a compound of Formula (Ia) with an epoxide of Formula (Ib):

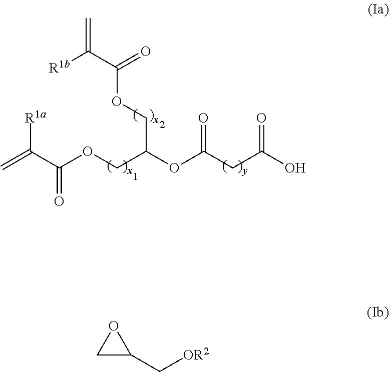

wherein:
R^{1a} and R^{1b} are, each independently, H, halogen, or C₁-C₆ alkyl; R² is H, C₁-C₂₀ alkyl, —C(═O)C₁-C₂₀ alkyl, C₁-C₆ haloalkyl, acrylate, silyl ether, or a moiety comprising a photoinitiator; and x₁, x₂, and y are each independently 1, 2, or 3.

In some embodiments, a method for synthesizing a compound having the structure of Formula (VI) is described. The method comprises reacting a compound of Formula (VIa) with an epoxide of Formula (Ib):

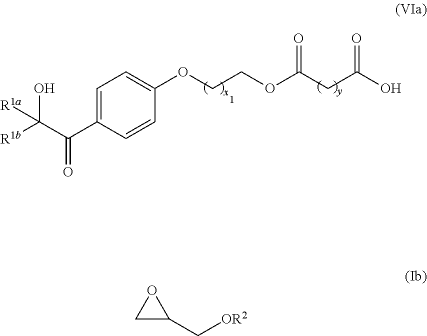

wherein:
R^{1a} and R^{1b} are, each independently, H, halogen, or C₁-C₆ alkyl; R² is H, C₁-C₂₀ alkyl, —C(═O)C₁-C₂₀ alkyl, C₁-C₆ haloalkyl, acrylate, silyl ether, or a moiety comprising a photoinitiator; and x₁ and y are each independently 1, 2, or 3.

In one embodiment, R^{1a} and R^{1b} are, each independently, H, halogen, or C₁-C₆ alkyl. In some embodiments, R^{1a} and R^{1b} are H. In some embodiments, R^{1a} and R^{1b} are halogen. In some embodiments, R^{1a} and R^{1b} are C₁-C₆ alkyl. In some embodiments, R^{1a} and R^{1b} are, each independently, H or C₁-C₃ alkyl. In some embodiments, R^{1a} and R^{1b} are, each independently, H or C₁ alkyl. In some specific embodiments, R^{1a} and R^{1b} are, each independently, H or —CH₃. In some other specific embodiments, R^{1a} or R^{1b} is H.

In one embodiment, R² is H, C₁-C₂₀ alkyl, —C(═O)C₁-C₂₀ alkyl, C₁-C₆ haloalkyl, acrylate, silyl ether, or a moiety comprising a photoinitiator. In some embodiments, R² is H. In some embodiments, R² is C₁-C₂₀ alkyl. In some embodiments, R² is C₁-C₁₈ alkyl. In some embodiments, R² is C₁-C₁₆ alkyl. In some embodiments, R² is C₆-C₂₀ alkyl. In some embodiments, R² is C₆-C₁₈ alkyl. In some specific embodiments, R² is C₆-C₁₆ alkyl. In some specific embodiments, R² is C₆-C₁₆ alkyl. In some embodiments, R² is C₁₄ alkyl. In some specific embodiments, R² is C₆-C₁₆ alkyl. In some embodiments, R² is Cis alkyl. In some embodiments, R² is C₁₆ alkyl. In some other specific embodiments, R² is C₆-C₁₆ alkyl. In some embodiments, R² is C₁₇ alkyl. In some other specific embodiments, R² is C₆-C₁₆ alkyl. In some embodiments, R² is C₁₈ alkyl. In some other specific embodiments, R² is C₆-C₁₆ alkyl. In some embodiments, R² is C₆ alkyl. In some other specific embodiments, R² is C₆-C₁₆ alkyl. In some embodiments, R² is C₇ alkyl. In some other specific embodiments, R² is C₆-C₁₆ alkyl. In some embodiments, R² is C₈ alkyl. In some other specific embodiments, R² is C₆-C₁₆ alkyl. In some embodiments, R² is C₉ alkyl. In some other specific embodiments, R² is C₆-C₁₆ alkyl. In some embodiments, R² is C₁₀ alkyl.

In some embodiments, R² is —C(═O)C₁-C₂₀ alkyl. In some embodiments, R² is —C(═O)C₁-C₁₈ alkyl. In some embodiments, R² is —C(═O)C₁-C₁₆ alkyl. In some embodiments, R² is —C(═O)C₁₂-C₂₀ alkyl. In some embodiments, R² is —C(═O)C₁₂-C₁₅ alkyl. In some embodiments, R² is —C(═O)C₁₂-C₁₆ alkyl. In some specific embodiments, R² is —C(═O)C₁₄ alkyl. In some specific embodiments, R² is —C(═O)C₁₅ alkyl. In some specific embodiments, R² is —C(═O)C₁₆ alkyl.

In some embodiments, R² is C₁-C₆ haloalkyl. In some embodiments, R² is C₂-C₄ haloalkyl. In some embodiments, R² is C₂ haloalkyl. In some embodiments, R² is C₃ haloalkyl. In some embodiments, R² is C₄ haloalkyl. In some embodiments, R² is C₄ fluoroalkyl. In some embodiments, R² is C₄ di, tri, or tetra-fluoroalkyl. In some embodiments, R² is C₄ tetra-fluoroalkyl. In some specific embodiments, R² is 1,1,2,2-tetrafluoropropyl.

In some embodiments, R² is acrylate. The acrylate of R² has the following structure:

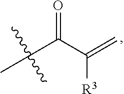

wherein R³ is H, halogen, or C₁-C₆ alkyl. In some embodiments, R³ is H, halogen, or C₁-C₃ alkyl. In some specific embodiments, R³ is H, halogen, or C₁ alkyl. In some embodiments, R³ is H. In some embodiments, R³ is halogen. In some embodiments, R³ is C₁-C₃ alkyl. In some embodiments, R³ is C₁ alkyl. In some embodiments, R³ is a methyl (—CH₃). In some embodiments, R³ is C₂ alkyl. In some embodiments, R³ is C₃ alkyl.

In some embodiments, R² is silyl ether. The silyl ether of R² has the following structure:

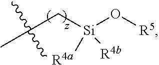

wherein R^{4a} and R^{4b} are, each independently H or C₁-C₆ alkyl; R⁵ is H, C₁-C₆ alkyl, or silyl; and z is an integer from 1 to 5. In some embodiments, R^{4a} and R^{4b} are, each independently H or C₁-C₄ alkyl. In some embodiments, R^{4a} and R^{4b} are, each independently H or C₁-C₃ alkyl. In some specific embodiments, R^{4a} and R^{4b} are, each independently H or C₁ alkyl. For example, R^{4a} and R^{4b} are, each independently H or a methyl (—CH₃).

In some embodiments, R⁵ is H, C₁-C₄ alkyl, or silyl. In some specific embodiments, R⁵ is silyl. In some specific embodiments, the silyl of R⁵ is dialkyl silyl. In some specific embodiments, the silyl of R⁵ is dimethyl silyl.

In some embodiments, z is an integer from 1 to 4. In some embodiments, z is an integer of 1. In some embodiments, z is an integer of 2. In some embodiments, z is an integer of 3. In some embodiments, z is an integer of 4. In some embodiments, z is an integer of 5. In some embodiments, R² is acrylate or silyl ether.

In some embodiments, R² is a moiety comprising a photoinitiator. The photoinitiator refers to a molecule that creates reactive species such as free radicals, cations, or anions when exposed to radiation. In some embodiments, R² is a moiety comprising a chemical structure with a photoinitiator capability including azobisisobutyronitrile, benzyl peroxide, 2,2-dimethoxy-2-phenylacetophenone, camphorquinone, BAPO Bis(2,4,6-trimethylbenzoyl)-phenylphosphineoxide, or the like. For instance, in some embodiments, R² is a moiety including a part of the following photoinitiator structures: azobisisobutyronitrile, benzyl peroxide, 2,2-dimethoxy-2-phenylacetophenone, camphorquinone, and BAPO Bis(2,4,6-trimethylbenzoyl)-phenylphosphineoxide. In some specific embodiments, the photoinitiator or R² has the following structure:

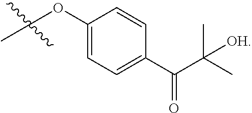 [SMILES: *Oc1ccc(C(=O)C(C)(C)O)cc1]

In some embodiments, R² has one of the following structures:

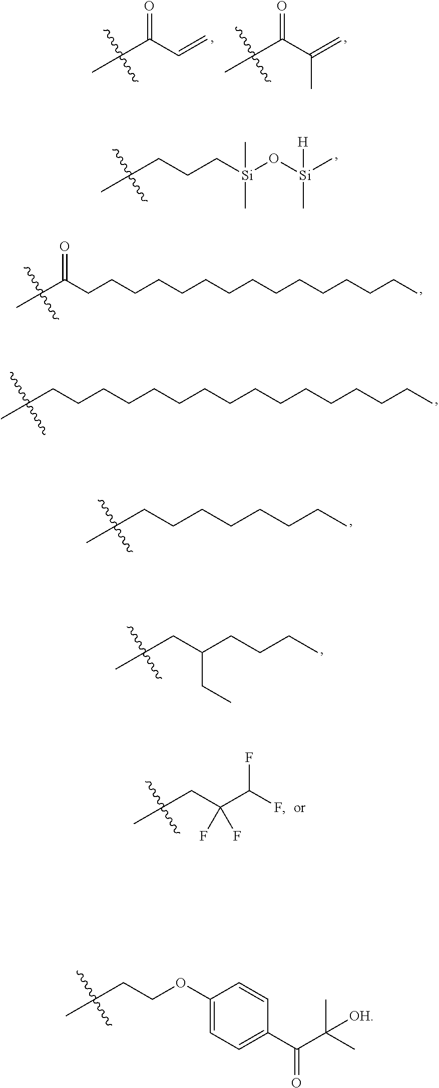

In some embodiments, R² is

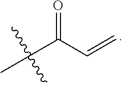 [SMILES: *C(=O)C=C]

In some embodiments, R² is

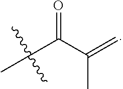 [SMILES: *C(=O)C(=C)C]

In some embodiments, R² is

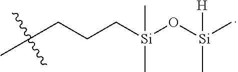 [SMILES: *CCC[Si](C)(C)O[SiH](C)C]

In some embodiments, R² is

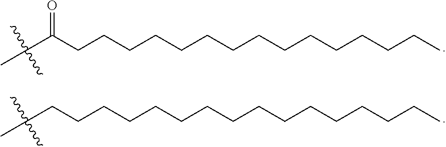 [SMILES: *C(=O)CCCCCCCCCCCCCCC ; *CCCCCCCCCCCCCCCC]

In some embodiments, R² is
In some embodiments, R² is

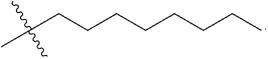 [SMILES: *CCCCCCCC]

In some embodiments, R² is

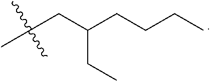 [SMILES: *CC(CC)CCCC]

In some embodiments, R² is

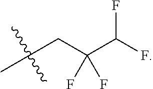 [SMILES: *CC(F)(F)C(F)F]

In some embodiments, R² is

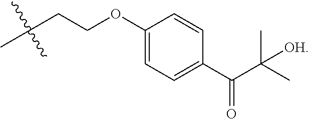 [SMILES: *CCOc1ccc(C(=O)C(C)(C)O)cc1]

In some embodiments, x₁, x₂, and y are, each independently, 1, 2 or 3. In some embodiments, x₁, x₂, and y are, each independently, 1 or 2. In some specific embodiments, x₁ and x₂ are 1. In some embodiments, y is 2.

In some embodiments, a molar ratio of the compound of Formula (Ia) to the epoxide of Formula (Ib) is from 1:1 to 1:1.2. In some embodiments, a molar ratio of the compound of Formula (Ia) to the epoxide of Formula (Ib) is from 1:1 to 1:1.1. In some specific embodiments, a molar ratio of the compound of Formula (Ia) to the epoxide of Formula (Ib) is from 1:1.04 to 1:1.08.

In some embodiments, a molar ratio of the compound of Formula (VIa) to the epoxide of Formula (Ib) is from 1:1 to 1:1.2. In some embodiments, a molar ratio of the compound of Formula (VIa) to the epoxide of Formula (Ib) is from 1:1 to 1:1.1. In some specific embodiments, a molar ratio of the compound of Formula (VIa) to the epoxide of Formula (Ib) is from 1:1.04 to 1:1.08.

In some embodiments, the method further comprising adding A) 4-methoxyphenol and triphenylphosphine; or B) 4-methoxyphenol and triethylamine.

In some embodiments, the compound, the epoxide, 4-methoxyphenol, and triphenylphosphine are stirred at 80° C. In some embodiments, wherein the compound of Formula (I) is formed in a quantitative yield.

In some embodiments, the oligomer compound has the following structure:

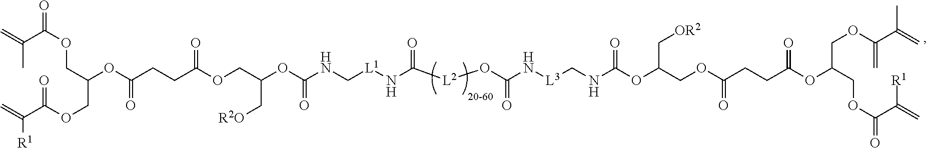

wherein:
L¹ and L³ are independently C₁-C₆ alkylene, 1-6 membered heteroalkylene, optionally substituted C₃-C₈ cycloalkylene, 4-8 membered heterocyclyl; and L² is C₁-C₆ alkylene or C₁-C₆ heteroalkylene.

In some embodiments, L¹ and L³ are independently substituted C₃-C₈ cycloalkylene or 4-8 membered heterocyclyl. In some embodiments, wherein L¹ and L³ are independently substituted C₆ cycloalkylene or 4-8 membered heterocyclyl. In some embodiments, L¹ and L³ are substituted cyclohexyl. In some specific embodiments, L¹ and L³ are trimethyl cyclohexyl.

In some embodiments, L² is C₃-C₅ heteroalkylene. In some embodiments, L² is C₄ heteroalkylene. In some specific embodiments, L² is butyloxy.

In some embodiments, the oligomer compound has one of the following structures:

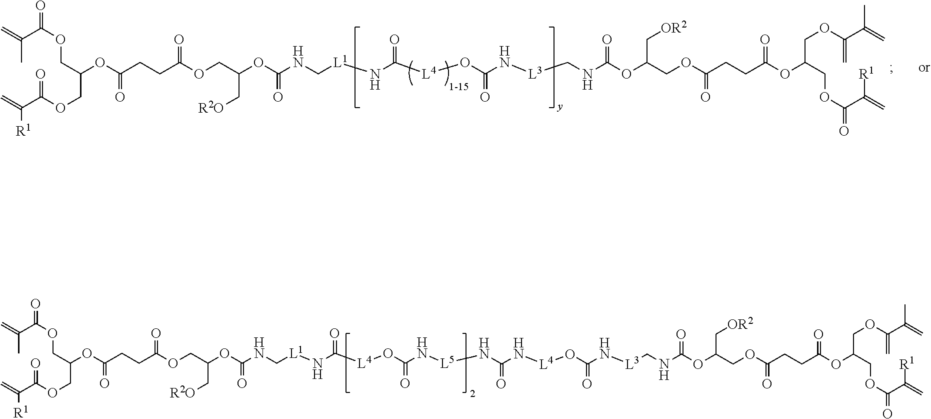

wherein:
L¹ and L³ are independently C₁-C₆ alkylene, 1-6 membered heteroalkylene, optionally substituted C₃-C₈ cycloalkylene, 4-8 membered heterocyclyl;
L⁴ is, each occurrence independently C₁-C₆ heteroalkylene; and
L⁵ is, each occurrence independently C₃-C₁₀ alkylene.

TABLE 2
Exemplary Oligomers

| Com- | |
|---|---|
| pound | |
| ID | Structure |
| II-1 | 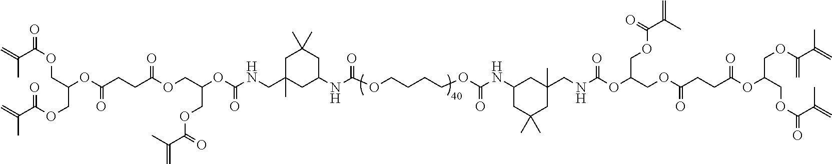 |
| II-2 | 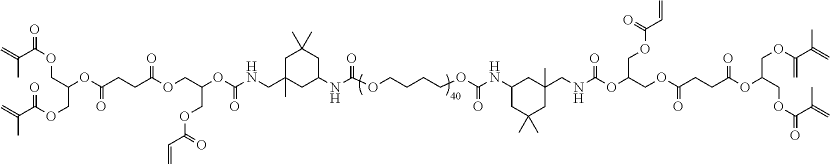 |
| II-3 | 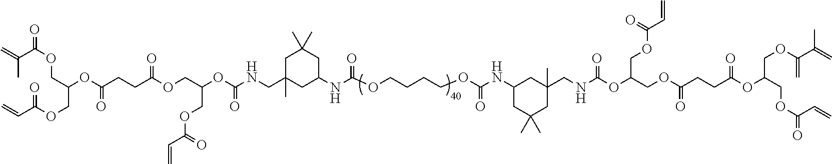 |
| II-4 | 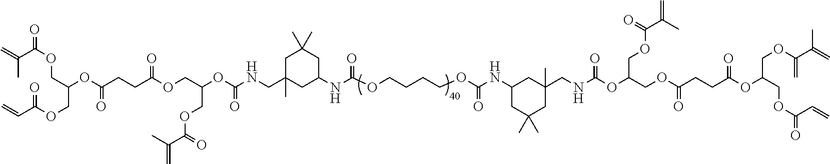 |
| III-1 | 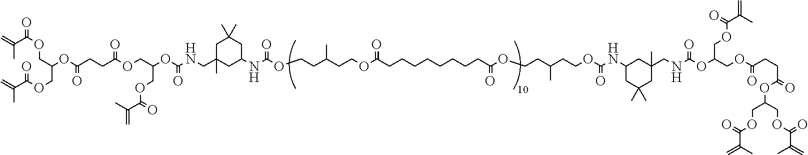 |
| IV-1 | 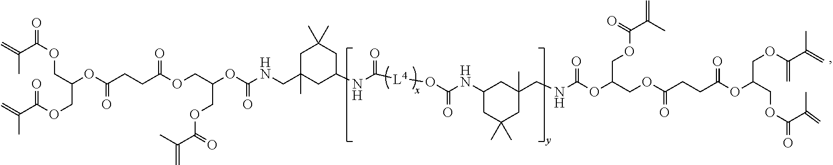 |
| | wherein: |
| | L⁴ has the following structure: |
| | 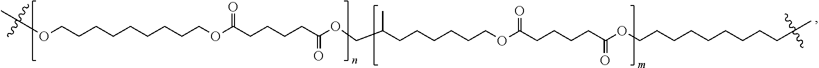 |
| | n + m is an integer of 7; |
| | x is an integer of 1; and |
| | y is an integer of 2. |
| IV-2 | 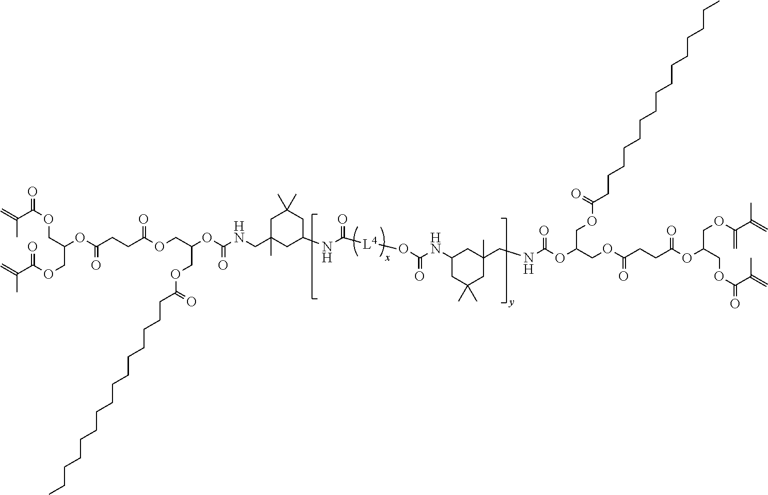 |
| | wherein: |
| | L⁴ has the following structure: |
| | 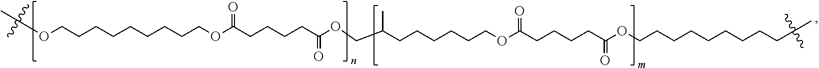 |
| | n + m is an integer of 7; |
| | x is an integer of 1; and |
| | y is an integer of 2. |
| IV-3 | 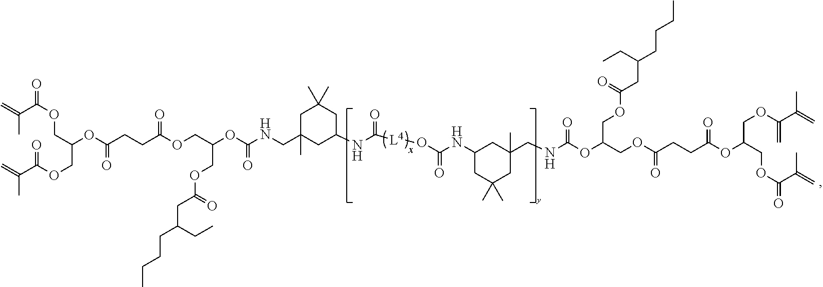 |
| | wherein: |
| | L⁴ has the following structure: |
| | 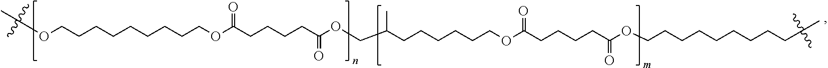 |
| | n+m is an integer of 7; |
| | x is an integer of 1; and |
| | y is an integer of 2. |
| V-1 | 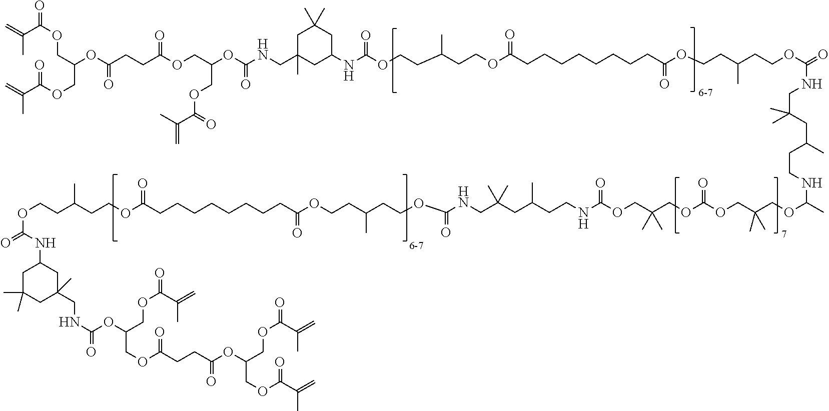 |
| VII-1 | 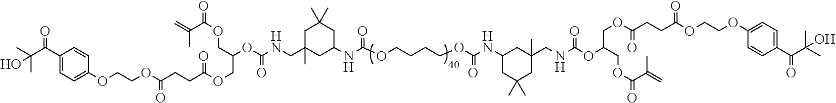 |

### Polymerizable Compositions

The present disclosure provides polymerizable compositions (also referred to as “curable resins”) that can comprise a plurality (e.g., >1) of polymerizable components such as the oligomer described in the present disclosure. A curable resin herein can be a photo-curable resin, a thermo-curable resin, or a combination thereof. As described herein, such polymerizable components can include one or more species of polymerizable compounds of the present disclosure (e.g., 1, 2, 3, or more different species), one or more species of polymerizable monomers (e.g., reactive diluents), and one or more species of telechelic oligomers and/or polymers (e.g., toughness modifiers). The curable resins provided herein can comprise lower amounts (e.g., per weight or volume) of polymerizable monomers (e.g., reactive diluents) compared to conventional resins, and instead contain one or more species of polymerizable compounds of the present disclosure. In some embodiments, however, no or only low amounts (e.g., 5% w/w or less) of a reactive diluent may be used. Resins provided herein can form polymeric materials with advantageous mechanical properties, reduced leaching of (e.g., unreacted) resin components (e.g., monomers) from the cured material, and an increased phase separation while providing a more continuous and uniform polymer matrix.

In some embodiments, a thiol-ene (and variations) is the oligomer cures without oxygen inhibition. In some embodiments, a poly-ene material is suitable to form a thiol-ene material (i.e., a hydrophobic component). Suitable poly-ene material may include acrylates, methacrylates, vinyl ethers, allyl ethers, allyl silane, vinyl silane, aliphatic enes, allyl esters, allyl carbonates, vinyl esters, acrylamides, vinyl carbonates, and combinations thereof. In some embodiments, the poly-ene is a primary aliphatic ene, an allyl ether, a vinyl ether, an allyl silane, a vinyl silane, or a combination thereof. In some embodiments, polybutadiene (e.g., 1,2 addition) is the hydrophobic component. In some embodiments, the polybutadiene has multiple molecular weights (i.e., it exists as a plurality of molecules with a range of molecular weights). In some embodiments, the hydrophobic material is an allyl silane, a vinyl silane, or a combination thereof. In some embodiments, the allyl silane and/or vinyl silane is telechelic or pendent.

In some embodiments, the thiol component of the oligomer is formed from a primary, secondary, and/or tertiary thiol. In some embodiments, the thiol is formed from a primary or secondary thiol. In some embodiments, the thiol is an aliphatic thiol having 4-10 carbons (e.g., 6 or more, 8 or more, etc.) or more.

In some embodiments, the oligomer is a thiol formed from the reaction product of smaller thiols with vinyl ethers, aliphatic enes, allyl ethers, epoxides, and other reactive groups. In some embodiments, the formation is an easy way to create higher molecular weight thiols with reduced vapor pressure. In some embodiments, siloxane-based monomers, oligomers, and polymers with thiol functionality are present as a component of the polymerizable composition. In some embodiments, the polymerizable composition uses all siloxane-based components as the oligomer. In some embodiments, the siloxane-based components comprise phenylmethyl siloxane, diphenyl siloxane, or combinations thereof. In some embodiments, the siloxane-based component imparts solubility to the polymerizable composition.

In some embodiments, a hybrid formulation with blocks of siloxane and organic components are also useful for building compatibility. In some embodiments, the hybrid formulation includes fluoropolymers. In some embodiments, a fully fluoropolymer is included, or a hybrid organic and fluoropolymer component is used.

In some embodiments, the polymerizable composition or polymer coating comprises particles. In some embodiments, particles are reactive. In some embodiments, particles are non-reactive. In some embodiments, particles are organic (e.g., carbon-based). In some embodiments, particles are inorganic (e.g., non-carbon based).

In some embodiments, organic particles comprise crystalline materials, crosslinked materials, thermoplastic materials, solidified resin mixes, or other organic materials. In some embodiments, inorganic particles comprise ceramics, minerals, metals, or others. In some embodiments, silicon, titanium, and aluminum based inorganic materials are included as silicon, titanium, and aluminum oxides, and mixtures thereof. In some embodiments, the particles are formed in place after the polymerizable composition or polymer coating is applied—for example: condensation of silanols in the presence moisture.

In some embodiments, form factors for the particles comprise platelets, fibers, irregular shapes, spherical, and others and include various aspect ratios. In some embodiments, platelets have diameters greater than thickness by greater than 2:1, greater than 5:1, or greater than 10:1. In some embodiments, particles can range in size (largest dimension) from 10 nm to 500 microns, but typically are from 100 nm to 200 microns, 10 microns to 150 microns, 30 microns to 100 microns. In some embodiments, particles are present as mixtures of different sizes, have large size distributions, or both. In other embodiments, particles have a low size distribution.

In some embodiments, layering is present within the polymer coating. That is, in some embodiments, the polymer composition is applied in multiple layers. In some embodiments, the polymerizable composition includes different mixtures of organic/inorganic materials with surface active materials. In some embodiments, the surface-active materials can phase separate from the rest of the polymerizable composition as the polymer coating is formed. In some embodiments, the polymer coating is formed as a layer or layers within the coating. In some embodiments, the polymer coating comprises layers on the surface of the polymer coating. In some embodiments, the polymer coating comprises a layer or layers within the coating and layers on the surface of the polymer coating. Such separation into layers can occur during or after polymer coating application. In some embodiments, layers include siloxane, silane, or fluorocarbon materials. In some embodiments, layers include N-silylamines. In some embodiments, the N-silylamines readily reaction with alcohols and silanols and quickly form a reacted skin on the polymer coating. In some embodiments, the polymerization composition includes silane coupling agents (and similarly designed materials) that impart improved adhesion of the polymer coating. In some embodiments, a layer the surface interface assists with adhesion or reaction of the coating to the object.

In some embodiments, the polymerizable composition comprises a thermal initiator, a photoinitiators, or both. In some embodiments, the composition does not include any initiator. In some embodiments, the polymerizable composition is treating with an activating agent such as heat or light. In some embodiments, the initiator increases the speed of curing step. In some embodiments, the initiator is present at a low concentration, for instance, less than 1 wt %, less than 0.5 wt %, less than 0.025 wt %, or less than 0.1 wt %.

In some embodiments, short wavelengths of UV are used to initiate a polymerization. In some embodiments, a reaction of thiol-enes when no initiators are used can be initiated with UV light at less than 400 nm, less than 375 nm, less than 360 nm, less than 350 nm, less than 330 nm, less than 300 nm. When photoinitiators are used, any wavelength to which the photoinitiator is sensitive to can be used and includes the whole visible spectrum and even into the IR spectrum. Thermal initiators can be activated with various forms of heat, including infrared, hot air, hot gas (e.g., in an oven), hot liquids, ultrasonic energy, and direct contact with hot materials. Typically, thermal polymerizations are performed at temperatures above 60° C., and more preferably above 80° C., or 100° C., as such formulations have higher shelf and vat lives. However, in some embodiments, the curing step is performed at room temperature.

Coating adhesion is facilitated by providing functional groups that bond with the article or object being coated. In some embodiments, the bonding is polar, dipole, hydrogen, pi-pi stacking, ionic, ligand, coordination, covalent bonding, or combinations thereof. In some embodiments, the bonding is covalent. Covalent bonding is facilitated by using functional groups that react with functional groups on the surface of the article or object. In some embodiments, amines, enes, epoxides, cyclic esters, cyclic ethers, thiols, hydroxyls, isocyanates, silanols, and other functional groups form covalent bonds with an article or object. In some embodiments, the formation of covalent bonds occurs upon contact of the polymerizable composition to the article or object. In certain embodiments, the bonding occurs at a later time after the polymerizable composition contacts the article or object (e.g., minutes later, hours later, or not until upon activation with an energy source such as heat, light, or other). In some embodiments, the bonding is initiated when the coated article or object is exposed to a reagent (e.g., water, oxygen, or amine) or exposed to a catalyst (via air or sprayed or liquid dipped). In some embodiments, the reaction the forms covalent bonds are condensation of silanols, related titanates, or combinations thereof. In some embodiments, the condensation is initiated using room temperature vulcanization) methods.

In some embodiments, the polymer coating has a thickness from 1 to 10 microns, 5 to 30 microns, 10 to 100 microns, 50 to 200 microns. In some embodiments, coatings are applied using various methods such as spray, electrostatic spray, fog, dip, or combinations thereof. In some embodiments, fog coatings are useful in that the fog can be created by pressure and a small nozzle or with an ultrasonic humidifier. In such fogs, the article to be coated in placed in the fog. In some embodiments, the article or object can be rotated in different orientations to facilitate even coverage and/or blowers can move the fog around to part. In some embodiments, such methods can be useful for obtaining an even coating onto the article or object by varying the amount of time spent in the fog as well as the density of the fog (droplet concentration) as well as the size of the droplets. In some embodiments, fog and ultrasonic methods require low viscosity to form small droplets.

In some embodiments, dip coatings are used and can involve low viscosity liquids that are easy to fling off or spin off the excess liquid. In some embodiments, a polymerizable composition is heated as a liquid to reduce the viscosity. In some embodiments, the method includes steps of controlling the viscosity of the polymerizable composition as a liquid as a means for controlling the thickness of the initial liquid layer that stays with the part when removed from the dip bath (e.g., the polymer coating).

In some embodiments, the polymerizable composition is heated and the article or object to be coated is cooled. In some embodiments, when the article or object is dipped into the polymerizable composition a layer of the polymerizable composition/polymer coating forms at or on the surface of the article or object. In some embodiments, the thickness of the polymer coating is controlled using a variety of methods including controlling the time spent in the polymerizable composition (e.g., the dipping time), the temperature of the polymerizable composition, the temperature of the article or object. Similarly, parameters for other techniques (e.g., spray coating, fog coating) are modified to achieve desirable results. For example, in some embodiments, excess polymerizable composition (e.g., as a liquid) can be centrifuged off the object or left on depending on the amount left on the part and the desired coating thickness. In some embodiments, freeze coatings are used for very low viscosity liquids as most of the unfrozen liquid can run off very quick and may not require spinning or shaking of excess liquid.

Some embodiments do not require shaking or flinging excess coating liquid off the article or object. Other embodiments do require such action to obtain a uniform coating. In some embodiments, centrifugal methods are particularly useful when performed at different angles or orientations of the object or by a multiaxial centrifuge. In some embodiments, the G-Force needed is dependent of the viscosity and/or temperature. In some embodiments, the G-force applied, the time of spinning, and the temperature are used to control the amount of polymerizable composition and thus the thickness of the polymer coating that remains on the article or object.

In some embodiments, a freeze coating method is used, and a cold solvent spray or dip/wash is be used to remove any non-solidified coating (temperature of the solvent is near, but below, the melting temperature of the solidified coating to prevent it from being removed while the liquid portion of the coating is removed. In some embodiments, the solvent is evaporated, and the coating material recovered for reuse.

In some embodiments, a method for coating an object or article includes one or more of the following steps:
an object or article (pre or post cure) is coated;
the excess coating liquid is removed;
the coating is allowed to cure;
the object or article is laser marked or marked with an ink jet printer with identification marking; and/or
a solvent wash, extraction, and/or sterilization step occurs.

In some embodiments, the order of operations can be different than the above.

In some embodiments, the coating is selected such that it imparts the following characteristics to the surface of the object or article: hydrophobic, neutral, or hydrophilic, transparent or opaque, colored or colorless, low surface energy, neutral, or high surface energy, stainable or stain resistant.

In some embodiments, the coating imparts wear resistance, scratch resistance, stain resistance, barrier properties, color, ease of laser marking or ink jetting, antifouling, antibacterial, antifungal, antimicrobial, pharmacological, release of compounds, odor prevention, closing of pores, gloss surface, matte surface, slick surface, gripping surface, etc.

In various cases, additional reactive diluents may be present and can comprise an acrylate or methacrylate moiety for incorporation into an polymeric backbone, coupled to a linear or cyclic (e.g., mono-, bi-, or tricyclic) side-chain moiety. Generally, any aliphatic, cycloaliphatic, or aromatic molecule with a mono-functional polymerizable reactive functional group can be used (also includes liquid crystalline monomers). In some instances, the polymerizable reactive functional groups is an acrylate or methacrylate group. In some instances, an additional polymerizable monomer is a syringol, guaiacol, or vanillin derivative, e.g., homosalic methacrylate (HSMA), syringyl methacrylate (SMA), isobornyl methacrylate (IBOMA), isobornyl acrylate (IBOA), etc. A reactive diluent used herein can have a low vapor pressure as further described below. In some embodiments, however, no or only low amounts (e.g., 5% w/w or less) of a reactive diluent may be used.

In some embodiments, the polymerizable composition is a photocurable composition. In certain embodiments, the polymerizable composition is a thermal curable composition. In some embodiments, the polymerizable composition is a combination of a photocurable composition and a thermal curable composition.

In some embodiments, the initiator comprises a photoinitiator. In some embodiments, the photoinitiator comprises a free radical photoinitiator. In certain embodiments, the initiator comprises a thermal initiator. In some embodiments, the thermal initiator comprises azobisisobutyronitrile, 2,2′-azodi(2-methylbutyronitrile), or a combination thereof.

In some embodiments, the concentration of reactive diluent ranges from 25-65 wt %. In certain embodiments, the concentration of reactive diluent ranges from 5-90 wt %, 5-80 wt %, 5-70 wt %, 5-60 wt %, 5-50 wt %, 5-40 wt %, 5-30 wt %, 5-20 wt %, 5-10 wt %, 10-70 wt %, 20-70 wt %, 30-70 wt %, 40-70 wt %, 50-70 wt %, 50-60 wt %, or 30-50 wt %. In some embodiments, the concentration of reactive diluent ranges from 30-60 wt %.

In some embodiments, the polymer composition comprises 10-80 wt % of reactive diluent.

In certain embodiments, the polymerizable composition comprises 0.01-10 wt % of the initiator. In some embodiments, the polymerizable composition comprises 0.05-10 wt %, 0.1-10 wt %, 0.5-10 wt %, 1.0-10 wt %, 5-10 wt %, 7.5-10 wt %, 0.01-5 wt %, 0.01-2 wt %, 0.01-1 wt %, or 0.01-0.5 wt % of the initiator.

In some embodiments, the polymerizable composition comprises 0.5-99.5 wt %, 1-99 wt %, 10-95 wt %, 20-90 wt %, 25-60 wt %, or 35-50 wt % of the monomer(s) or reactive diluent.

In certain embodiments, the polymerizable composition comprises a first reactive diluent, the polymerizable composition further comprises a second reactive diluent. In some embodiments, the first or second reactive diluent comprises an alkyl acrylate, an alkyl methacrylate, a homosalic acrylate, a homosalic methacrylate, or combinations thereof. In certain embodiments, the second polymerizable monomer is a homosalic acrylate, a homosalic methacrylate, or combinations thereof. In some embodiments, the polymerizable composition comprises 25-35 wt % of the first reactive diluent and 10-50% of the second reactive diluent.

In some embodiments, the polymerizable composition further comprises one or more reagents selected from the group consisting of a crosslinking modifier, a glass transition temperature modifier, a toughness modifier, a polymerization catalyst, a polymerization inhibitor, a light blocker, a plasticizer, a surface energy modifier, a pigment, a dye, a filler, a biologically significant chemical, a solvent, and combinations thereof.

In some embodiments, the polymerizable composition is capable of being 3D printed at a printing temperature greater than 25° C. In certain embodiments, the printing temperature is at least 30° C., 40° C., 50° C., 60° C., 80° C., or 100° C. In some embodiments, the polymerizable composition has a viscosity from 30 cP to 50,000 cP at a printing temperature. In certain embodiments, the printing temperature is from 25° C. to 150° C. In some embodiments, the polymerizable composition comprises less than 20 wt % hydrogen bonding units. In certain embodiments, the polymerizable composition is a liquid at a temperature from about 40° C. to about 100° C. In certain embodiments, the polymerizable composition is a liquid at a temperature of above about 40° C. with a viscosity less than about 20 PaS. In some embodiments, the polymerizable composition is a liquid at a temperature of above about 40° C. with a viscosity less than about 1 PaS. In some embodiments, at least a portion of the polymerizable composition melts at a temperature from about 60° C. to about 0° C.

In some embodiments, a reactive diluent of the present disclosure can have a low vapor pressure at an elevated temperature and a high boiling point. Such low vapor pressure can be particularly advantageous for use of such a reactive diluent in curable (e.g., photocurable) compositions and additive manufacturing where elevated temperatures (e.g., 60° C., 80° C., 90° C., or higher) may be used. In various instances, a reactive diluent can have a vapor pressure of at most about 12 Pa at 60° C. In various instances, a reactive diluent can have a vapor pressure of at most about 2 Pa to 10 Pa at 60° C. In various instances, a reactive diluent can have a vapor pressure of at most about 2 Pa to 5 Pa at 60° C. Thus, in some embodiments, a reactive diluent of the present disclosure can have a low mass loss at an elevated temperature. As used herein, a mass loss of a compound at a certain temperature (e.g., 90° C.) for a certain period (e.g., 2 hours) can be used as a measure for volatility of such compounds. Herein, “substantially no volatility” can refer to a mass loss<1 wt % at the respective temperature, e.g., at 90° C. for 2 hours. In various instances, a reactive diluent of the present disclosure can have a mass loss<1 wt % at the respective temperature at 90° C. after heating at that temperature for 2 hours. In some embodiments, a reactive diluent can have a mass loss of less than about 0.5% after heating at 90° C. for 2 h. In some embodiments, a reactive diluent can have a mass loss of about 0.1% to about 0.45% after heating at 90° C. for 2 h. In some embodiments, a reactive diluent can have a mass loss of about 0.05% to about 0.25% after heating at 90° C. for 2 h.

In various embodiments, a curable resin or polymerizable composition herein is a photo-curable resin. Such photo-curable resin described herein can further comprise one or more photoinitiators. Such photoinitiator, when activated with light of an appropriate wavelength (e.g., UV/VIS) can initiate a polymerization reaction (e.g., during photo-curing) between monomers and themselves and/or other potentially polymerizable components that may be present in the photo-curable resin, to form a polymeric material as further described herein. Generally, photoinitiators described in the present disclosure can include those that can be activated with light and initiate polymerization of the polymerizable components of the formulation. A “photoinitiator”, as used herein, may generally refer to a compound that can produce radical species and/or promote radical reactions upon exposure to radiation (e.g., UV or visible light).

In some embodiments, a photo-curable resin herein further comprises 0.05 to 1 wt %, 0.05 to 2 wt %, 0.05 to 3 wt %, 0.05 to 4 wt %, 0.05 to 5 wt %, 0.1 to 1 wt %, 0.1 to 2 wt %, 0.1 to 3 wt %, 0.1 to 4 wt %, 0.1 to 5 wt %, 0.1 to 6 wt %, 0.1 to 7 wt %, 0.1 to 8 wt %, 0.1 to 9 wt %, or 0.1 to 10 wt %, based on the total weight of the composition, of a photoinitiator. In some embodiments, the photoinitiator is a free radical photoinitiator. In certain embodiments, the free radical photoinitiator comprises an alpha hydroxy ketone moiety (e.g., 2-hydroxy-2-methylpropiophenone or 1-hydroxycyclohexyl phenyl ketone), an alpha-amino ketone (e.g., 2-benzyl-2-(dimethylamino)-4′-morpholinobutyrophenone or 2-methyl-1-[4-(methylthio)phenyl]-2-morpholinopropan-1-one), 4-methyl benzophenone, an azo compound (e.g., 4,4′-Azobis(4-cyanovaleric acid), 1,1′-Azobis(cyclohexanecarbonitrile, Azobisisobutyronitrile, 2,2′-Azobis(2-methylpropionitrile), or 2,2′-Azobis(2-methylpropionitrile)), an inorganic peroxide, an organic peroxide, or any combination thereof. In some embodiments, the composition comprises a photoinitiator comprising SpeedCure TPO-L (ethyl(2,4,6-trimethylbenzoyl)phenyl phosphinate).

In some embodiments, a photo-curable composition comprises a photoinitiator selected from a benzophenone, a mixture of benzophenone and a tertiary amine containing a carbonyl group which is directly bonded to at least one aromatic ring, and an Irgacure (e.g., Irgacure 907 (2-methyl-1-[4-(methylthio)-phenyl]-2-morpholino-propanone-1) or Irgacure 651 (2,2-dimethoxy-1,2-diphenylethan-1-one). In some embodiments, the photoinitiator comprises an acetophenone photoinitiator (e.g., 4′-hydroxyacetophenone, 4′0phenoxyacetophenone, 4′-ethoxyaceto-phenone), a benzoin, a benzoin derivative, a benzil, a benzil derivative, a benzophenone (e.g., 4-benzoylbiphenyl, 3,4-(dimethylamino)benzophenone, 2-methylbenzophenone), a cationic photoinitiator (e.g., diphenyliodonium nitrate, (4-iodophenyl)diphenylsulfonium triflate, triphenylsulfonium triflate), an anthraquinone, a quinone (e.g., camphorquinone), a phosphine oxide, a phosphinate, 9,10-phenanthrenequinone, a thioxanthone, any combination thereof, or any derivative thereof. In some embodiments, the photoinitiator is phenylbis(2,4,6-trimethylbenzoyl)phosphine oxide (BAPO).

In some embodiments, the photoinitiator can have a maximum wavelength absorbance from 200 to 300 nm, from 300 to 400 nm, from 400 to 500 nm, from 500 to 600 nm, from 600 to 700 nm, from 700 to 800 nm, from 800 to 900 nm, from 150 to 200 nm, from 200 to 250 nm, from 250 to 300 nm, from 300 to 350 nm, from 350 to 400 nm, from 400 to 450 nm, from 450 to 500 nm, from 500 to 550 nm, from 550 to 600 nm, from 600 to 650 nm, from 650 to 700 nm, or from 700 to 750 nm. In some embodiments, the photoinitiator has a maximum wavelength absorbance from 300 to 500 nm.

In some embodiments, a photo-curable resin of the present disclosure can further comprise a crosslinking modifier (e.g., in addition to a polymerizable compound disclosed herein that can act as a cross-linker, or in instances where the polymerizable compound does not act as a cross-linker), a light blocker, a solvent, a glass transition temperature modifier, or a combination thereof. In some embodiments, the photo-curable resin comprises 0-25 wt % of the crosslinking modifier, the crosslinking modifier having a number-average molecular weight equal to or less than 1,500 Da. In some embodiments, the photo-curable resin comprises from 0 to 10 wt %, from 0 to 9 wt %, from 0 to 8 wt %, from 0 to 7 wt %, from 0 to 6 wt %, from 0 to 5 wt %, from 0 to 4 wt %, from 0 to 3 wt %, from 0 to 2 wt %, from 0 to 1 wt %, or from 0 to 0.5 wt % of the light blocker. In some embodiments, the photo-curable resin comprises a solvent. In some embodiments, the solvent comprises a nonpolar solvent. In certain embodiments, the nonpolar solvent comprises pentane, cyclopentane, hexane, cyclohexane, benzene, toluene, 1,4-dioxane, chloroform, diethyl ether, dichloromethane, a derivative thereof, or a combination thereof. In some embodiments, the solvent comprises a polar aprotic solvent. In certain embodiments, the polar aprotic solvent comprises tetrahydrofuran, ethyl acetate, acetone, dimethylformamide, acetonitrile, DMSO, propylene carbonate, a derivative thereof, or a combination thereof. In some embodiments, the solvent comprises a polar protic solvent. In certain embodiments, the polar protic solvent comprises formic acid, n-butanol, isopropyl alcohol, n-propanol, t-butanol, ethanol, methanol, acetic acid, water, a derivative thereof, or a combination thereof. In some embodiments, the photo-curable resin comprises less than 90% of the solvent by weight.

In some embodiments, the added resin component (e.g., a crosslinking modifier, a polymerization catalyst, a polymerization inhibitor, a glass transition temperature modifier, a light blocker, a plasticizer, a solvent, a surface energy modifier, a pigment, a dye, a filler, or a biologically significant chemical) is functionalized so that it can be incorporated into the polymeric material so that it cannot readily be extracted from the final cured material. In certain embodiments, the polymerization catalyst, polymerization inhibitor, light blocker, plasticizer, surface energy modifier, pigment, dye, and/or filler, are functionalized to facilitate their incorporation into the cured polymeric material.

In some embodiments, a resin herein comprises a component in addition to a polymerizable compound described herein that can alter the glass transition temperature of the cured polymeric material. In such instances, a glass transition temperature modifier (also referred to herein as a T_{g} modifier or a glass transition modifier) can be present in a photo-curable composition from about 0 to 50 wt %, based on the total weight of the composition. The T_{g} modifier can have a high glass transition temperature, which leads to a high heat deflection temperature, which can be necessary to use a material at elevated temperatures. In some embodiments, the curable composition comprises 0 to 80 wt %, 0 to 75 wt %, 0 to 70 wt %, 0 to 65 wt %, 0 to 60 wt %, 0 to 55 wt %, 0 to 50 wt %, 1 to 50 wt %, 2 to 50 wt %, 3 to 50 wt %, 4 to 50 wt %, 5 to 50 wt %, 10 to 50 wt %, 15 to 50 wt %, 20 to 50 wt %, 25 to 50 wt %, 30 to 50 wt %, 35 to 50 wt %, 0 to 40 wt %, 1 to 40 wt %, 2 to 40 wt %, 3 to 40 wt %, 4 to 40 wt %, 5 to 40 wt %, 10 to 40 wt %, 15 to 40 wt %, or 20 to 40 wt % of a T_{g} modifier. In certain embodiments, the curable composition comprises 0-50 wt % of a glass transition modifier. In some instances, the number average molecular weight of the T_{g} modifier is 0.4 to 5 kDa. In some embodiments, the number average molecular weight of the T_{g} modifier is from 0.1 to 5 kDa, from 0.2 to 5 kDa, from 0.3 to 5 kDa, from 0.4 to 5 kDa, from 0.5 to 5 kDa, from 0.6 to 5 kDa, from 0.7 to 5 kDa, from 0.8 to 5 kDa, from 0.9 to 5 kDa, from 1.0 to 5 kDa, from 0.1 to 4 kDa, from 0.2 to 4 kDa, from 0.3 to 4 kDa, from 0.4 to 4 kDa, from 0.5 to 4 kDa, from 0.6 to 4 kDa, from 0.7 to 4 kDa, from 0.8 to 4 kDa, from 0.9 to 4 kDa, from 1 to 4 kDa, from 0.1 to 3 kDa, from 0.2 to 3 kDa, from 0.3 to 3 kDa, from 0.4 to 3 kDa, from 0.5 to 3 kDa, from 0.6 to 3 kDa, from 0.7 to 3 kDa, from 0.8 to 3 kDa, from 0.9 to 3 kDa, or from 1 to 3 kDa. A polymerizable compound of the present disclosure (which can, in some cases, act by itself as a T_{g} modifier) and a separate T_{g} modifier compound can be miscible and compatible in the methods described herein. When used in the subject compositions, the T_{g} modifier may provide for high T_{g} and strength values, sometimes at the expense of elongation at break. In some cases, a toughness modifier may provide for high elongation at break and toughness via strengthening effects In some embodiments, the polymerizable composition comprises high amounts of toughness modifiers, while maintaining high values for strength and T_{g}.

### Resin Properties

A curable (e.g., photo-curable) resin (also referred to as a “polymerizable composition”) herein can be characterized by having one or more properties. Hence, in some instances, a reactive diluent can reduce a viscosity of the polymerizable composition (e.g., a photo-curable resin). In such cases, a reactive diluent can reduce the viscosity of the curable resin or polymerizable composition by at least about 5% compared to a composition that does not comprise the reactive diluent. In some instances, a reactive diluent can reduce the viscosity of a polymerizable composition by at least about 5%, 10%, 20%, 30%, 40%, or 50%. In some instances, a polymerizable composition of this disclosure can have a viscosity from about 30 cP to about 50,000 cP at a printing temperature. In some embodiments, the polymerizable composition has a viscosity less than or equal to 30,000 cP, less than or equal to 25,000 cP, less than or equal to 20,000 cP, less than or equal to 19,000 cP, less than or equal to 18,000 cP, less than or equal to 17,000 cP, less than or equal to 16,000 cP, less than or equal to 15,000 cP, less than or equal to 14,000 cP, less than or equal to 13,000 cP, less than or equal to 12,000 cP, less than or equal to 11,000 cP, less than or equal to 10,000 cP, less than or equal to 9,000 cP, less than or equal to 8,000 cP, less than or equal to 7,000 cP, less than or equal to 6,000 cP, or less than or equal to 5,000 cP at 25° C. In some embodiments, the polymerizable composition has a viscosity less than 15,000 cP at 25° C. In some embodiments, the polymerizable composition has a viscosity less than or equal to 100,000 cP, less than or equal to 90,000 cP, less than or equal to 80,000 cP, less than or equal to 70,000 cP, less than or equal to 60,000 cP, less than or equal to 50,000 cP, less than or equal to 40,000 cP, less than or equal to 35,000 cP, less than or equal to 30,000 cP, less than or equal to 25,000 cP, less than or equal to 20,000 cP, less than or equal to 15,000 cP, less than or equal to 10,000 cP, less than or equal to 5,000 cP, less than or equal to 4,000 cP, less than or equal to 3,000 cP, less than or equal to 2,000 cP, less than or equal to 1,000 cP, less than or equal to 750 cP, less than or equal to 500 cP, less than or equal to 250 cP, less than or equal to 100 cP, less than or equal to 90 cP, less than or equal to 80 cP, less than or equal to 70 cP, less than or equal to 60 cP, less than or equal to 50 cP, less than or equal to 40 cP, less than or equal to 30 cP, less than or equal to 20 cP, or less than or equal to 10 cP at a printing temperature. In some embodiments, the polymerizable composition has a viscosity from 50,000 cP to 30 cP, from 40,000 cP to 30 cP, from 30,000 cP to 30 cP, from 20,000 cP to 30 cP, from 10,000 cP to 30 cP, or from 5,000 cP to 30 cP at a printing temperature. In some embodiments, the printing temperature is from 0° C. to 25° C., from 25° C. to 40° C., from 40° C. to 100° C., or from 20° C. to 150° C. In some embodiments, the polymerizable composition has a viscosity from 30 cP to 50,000 cP at a printing temperature, wherein the printing temperature is from 20° C. to 150° C. In yet other embodiments, the polymerizable composition has a viscosity less than 20,000 cP at a print temperature. In some embodiments, the print temperature is from 10° C. to 200° C., from 15° C. to 175° C., from 20° C. to 150° C., from 25° C. to 125° C., or from 30° C. to 100° C. In preferred embodiments, the print temperature is from 20° C. to 150° C.

A polymerizable composition of the present disclosure can be capable of being 3D printed at a temperature greater than 25° C. In some cases, the printing temperature is at least about 30° C., 40° C., 50° C., 60° C., 80° C., or 100° C. As described herein, a photo-polymerizable monomer of this disclosure that can part of the polymerizable composition, can have a low vapor pressure and/or mass loss at the printing temperature, thereby providing improved printing conditions compared to conventional resins used in additive manufacturing.

In some embodiments, a polymerizable composition herein has a melting temperature greater than room temperature. In some embodiments, the polymerizable composition has a melting temperature greater than 20° C., greater than 25° C., greater than 30° C., greater than 35° C., greater than 40° C., greater than 45° C. greater than 50° C., greater than 55° C., greater than 60° C., greater than 65° C., greater than 70° C., greater than 75° C., or greater than 80° C. In some embodiments, the polymerizable composition has a melting temperature from 20° C. to 250° C., from 30° C. to 180° C., from 40° C. to 160° C., or from 50° C. to 140° C. In some embodiments, the polymerizable composition has a melting temperature greater than 60° C. In other embodiments, the polymerizable composition has a melting temperature from 80° C. to 110° C. In some instances, a polymerizable composition can have a melting temperature of about 80° C. before polymerization, and after polymerization, the resulting polymeric material can have a melting temperature of about 100° C.

In certain instances, it may be advantageous that a polymerizable composition is in a liquid phase at an elevated temperature. As an example, a conventional polymerizable composition can comprise polymerizable components that may be viscous at a process temperature, and thus can be difficult to use in the fabrication of objects (e.g., using 3D printing). As a solution for that technical problem, the present disclosure provides polymerizable composition comprising photo-polymerizable components such as monomers described herein that can melt at an elevated temperature, e.g., at a temperature of fabrication (e.g., during 3D printing), and can have a decreased viscosity at the elevated temperature, which can make such resin more applicable and usable for uses such as 3D printing. Hence, in some embodiments, provided herein are polymerizable compositions that are a liquid at an elevated temperature. In some embodiments, the elevated temperature is at or above the melting temperature (Tm) of the polymerizable composition. In certain embodiments, the elevated temperature is a temperature in the range from about 40° C. to about 100° C., from about 60° C. to about 100° C., from about 80° C. to about 100° C., from about 40° C. to about 150° C., or from about 150° C. to about 350° C. In some embodiments, the elevated temperature is a temperature above about 40° C., above about 60° C., above about 80° C., or above about 100° C. In some embodiments, a polymerizable composition herein is a liquid at an elevated temperature with a viscosity less than about 50 PaS, less than 2 about 0 PaS, less than about 10 PaS, less than about 5 PaS, or less than about 1 PaS. In some embodiments, a polymerizable composition herein is a liquid at an elevated temperature of above about 40° C. with a viscosity less than about 20 PaS. In yet other embodiments, a polymerizable composition herein is a liquid at an elevated temperature of above about 40° C. with a viscosity less than about 1 PaS.

In some embodiments, at least a portion of a polymerizable composition herein has a melting temperature below about 100° C., below about 90° C., below about 80° C., below about 70° C., or below about 60° C. In some embodiments, at least a portion of a polymerizable composition herein melts at an elevated temperature from about 100° C. and about 20° C., from about 90° C. and about 20° C., from about 80° C. and about 20° C., from about 70° C. and about 20° C., from about 60° C. and about 20° C., from about 60° C. and about 10° C., or from about 60° C. and about 0° C.

In various embodiments, a polymerizable composition herein as well as its photo-polymerizable components can be biocompatible, bioinert, or a combination thereof. In various instances, the photo-polymerizable compounds of a resin herein can have biocompatible and/or bioinert metabolic (e.g., hydrolysis) products.

A polymerizable composition of the present disclosure can comprise less than about 20 wt % or less than about 10 wt % hydrogen bonding units. In some embodiments, a polymerizable composition herein comprises less than about 15 wt %, less than about 10 wt %, less than about 9 wt %, less than about 8 wt %, less than about 7 wt %, less than about 6 wt %, less than about 5 wt %, less than about 4 wt %, less than about 3 wt %, less than about 2 wt %, or less than about 1 wt % hydrogen bonding units.

### Polymer or Polymeric Materials

The present disclosure provides polymeric materials (e.g., a polymeric coating). Such polymeric materials can be generated by curing a curable polymerizable composition described herein. A polymeric material provided herein can be biocompatible, bioinert, or a combination thereof. In various instances, a polymeric material herein is generated by photo-curing a photo-curable composition described herein. Such photo-curable compositions can comprise one or more polymerizable compounds of the present disclosure.

In some embodiments, a polymerizable composition herein can be cured by exposing such a composition or resin to electromagnetic radiation of appropriate wavelength. In various embodiments, the present disclosure provides a polymeric material (e.g., a polymeric coating) that can comprise one or more polymeric phases, wherein at least one polymeric phase of the one or more polymeric phases is a crystalline phase. In various embodiments, the present disclosure provides a polymeric material that can comprise one or more polymeric phases, wherein at least one polymeric phase of the one or more polymeric phases is an amorphous phase.

In some embodiments, the polymeric material (e.g., a polymeric coating) comprises one or more polymer types that may have formed, during curing, from the polymerizable compounds/compositions, telechelic polymers, oligomers, polymerizable monomers/reactive diluents, and/or any other polymerizable component. In some instances, such one or more polymer types can include one or more of comprises a homopolymer, a linear copolymer, a block copolymer, an alternating copolymer, a periodic copolymer, a statistical copolymer, a random copolymer, a gradient copolymer, a branched copolymer, a brush copolymer, a comb copolymer, a dendrimer, or any combination thereof. In some cases, the polymeric material comprises a random copolymer. In some embodiments, the polymeric material can comprise poly(ethylene) glycol (PEG), poly(ethylene) glycol diacrylate, PEG-THF, polytetrahydrofuran, poly(tert-butyl acrylate), poly(ethylene-co-maleic anhydride), any derivative thereof, or any combination thereof.

In some embodiments, the polymeric material (e.g., a polymeric coating) comprises an acrylate, an acrylamide, a methacrylamide, an acrylonitrile, a bisphenol acrylate, a carbohydrate, a fluorinated acrylate, a maleimide, an acrylate, 4-acetoxyphenethyl acrylate, acryloyl chloride, 4-acryloylmorpholine, 2-(acryloyloxy)ethyl]-trimethylammonium chloride, 2-(4-benzoyl-3-hydroxyphenoxy)ethyl acrylate, benzyl 2-propylacrylate, butyl acrylate, tert-butyl acrylate, 2[(butylamino)carbonyl]-oxy]ethyl acrylate, tert-butyl 2-bromoacrylate, 2-carboxyethyl acrylate, 2-chloroethyl acrylate, 2-(diethylamino)-ethyl acrylate, di(ethylene glycol) ethyl ether acrylate, 2-(dimethylamino)ethyl acrylate, 3-(dimethylamino)propyl acrylate, dipentaerythriol penta-/hexa-acrylate, ethyl acrylate, 2-ethylacryloyl chloride, ethyl 2-(bromomethyl)acrylate, ethyl cis-(beta-cyano)acrylate, ethylene glycol dicyclopentenyl ether acrylate, ethylene glycol methyl ether acrylate, ethylene glycol phenyl ether acrylate, ethyl 2-ethylacrylate, 2-ethylexyl acrylate, ethyl 2-propylacrylate, ethyl 2-(trimethylsilylmethyl)acrylate, hexyl acrylate, 4-hydroxybutyl acrylate, 2-hydroxyethyl acrylate, 2-hydroxy-3-phenoxypropyl acrylate, hydroxypropyl acrylate, isobornyl acrylate, isobutyl acrylate, isodecyl acrylate, isooctyl acrylate, lauryl acrylate, methyl 2-acetamidoacrylate, methyl acrylate, a methylene malonate (e.g., dibutyl methylene malonate, dihexyl methylene malonate, or dicyclohexyl methylene malonate), a methylene malonate macromerer (e.g, a polyester of 2-methylenemalonate such as Forza B3000 XP), methyl α-bromoacrylate, methyl 2-(bromo-methyl)acrylate, methyl 2-(chloromethyl)acrylate, methyl 3-hydroxy-2-methylenebutyrate, methyl 2-(trifluoromethyl)acrylate, octadecyl acrylate, pentabromobenzyl acrylate, penta-bromophenyl acrylate, pentafluorophenyl acrylate, poly(ethylene glycol) diacrylate, poly-(ethylene glycol) methyl ether acrylate, poly(propylene glycol) acrylate, epoxidized soybean oil acrylate, 3-sulfopropyl acrylate, tetrahydrofuryl acrylate, 2-tetrahydropyranyl acrylate, 3-(trimethoxysilyl)propyl acrylate, 3,5,5-trimethylhexyl acrylate, 10-undecenyl acrylate, urethane acrylate, urethane acrylate methacrylate, tricylcodecane diacrylate, isobornyl acrylate, a methacrylate, allyl methacrylate, benzyl methacrylate, (2-boc-amino)ethyl methacrylate, tert-butyl methacrylate, 9H-carbazole-9-ethylmethacrylate, 3-chloro-2-hydroxypropyl methacrylate, cyclohexyl methacrylate, 1,10-decamethylene glycol dimethacrylate, ethylene glycol dicyclopentenyl ether methacrylate, ethylene glycol methyl ether methacrylate, 2-ethylhexyl methacrylate, furfuryl methacrylate, glycidyl methacrylate, glycosyloxyethyl methacrylate, hexyl methacrylate, hydroxybutyl methacrylate, 2-hydroxy-5-N-methacrylamidobenzoic acid, isobutyl methacrylate, methacryloyl chloride, methyl methacrylate, mono-2-methacryloyloxy)ethyl succinate, 2-N-morpholinoethyl methacrylate, 1-naphthyl methacrylate, pentabromophenyl methacrylate, phenyl methacrylate, pentabromophenyl methacrylate, TEMPO methacrylate, 3-sulfopropyl methacrylate, triethylene glycol methyl ether methacrylate, 2-[(1′,1′,1′-trifluoro-2′-(trifluoromethyl)-2′Ohdroxy)propyl]-3-norbornyl methacrylate, 3,3,5-trimethylcyclohexyl methacrylate, (trimethylsilyl)methacrylate, vinyl methacrylate, isobornyl methacrylate, bisphenol A dimethacrylate, an Omnilane OC, tert-butyl acrylate, isodecyl acrylate, tricylcodecane diacrylate, a polyfunctional acrylate, N,N′-methylenebisacrylamide, 3-(acryloyloxy)-2-hydroxypropyl) methacrylate, bis[2-(methacryloyloxy)ethyl]phosphate, 1,3-butanediol diacrylate, 1,4-butanediol diacrylate, diurethane dimethacrylate, N,N′-ethylenebis(acrylamide), glycerol 1,3-diglycerolate diacrylate, 1,6-hexanediol diacrylate, hydroxypivalyl hydroxypivalate bis[6-(acryloyloxy)hexanoate], neopentyl glycol diacrylate, pentaerythritol diacrylate, 1,3,6-triacryloyl hexahydro-1,3,5-triazine, trimethlolpropane ethoxylate, tris[2-(acryloyloxy)ethyl]isocyanurate, any derivative thereof, or a combination thereof.

In some embodiments, the polymeric material (e.g., a polymeric coating) herein can comprise one or more reactive functional groups that can allow for further modification of the polymeric material, such as additional polymerization (e.g., post-curing). In some embodiments, polymeric material comprises a plurality of reactive functional groups, and the reactive functional groups can be located at one or both terminal ends of the polymeric material, in-chain, at a pendant (e.g., a side group attached to the polymer backbone), or any combination thereof. Non-limiting examples of reactive functional groups include free radically polymerizable functionalities, photoactive groups, groups facilitating step growth polymerization, thermally reactive groups, and/or groups that facilitate bond formation (e.g., covalent bond formation). In some embodiments, the reactive functional groups comprise an acrylate, a methacrylate, an acrylamide, a vinyl group, a vinyl ether, a thiol, an allyl ether, a norbornene, a vinyl acetate, a maleate, a fumarate, a maleimide, an epoxide, a ring-strained cyclic ether, a ring-strained thioether, a cyclic ester, a cyclic carbonate, a cyclic silane, a cyclic siloxane, a hydroxyl, an amine, an isocyanate, a blocked isocyanate, an acid chloride, an activated ester, a Diels-Alder reactive group, a furan, a cyclopentadiene, an anhydride, a group favorable toward photodimerization (e.g., an anthracene, an acenaphthalene, or a coumarin), a group that photodegrades into a reactive species (e.g., Norrish Type 1 and 2 materials), an azide, a derivative thereof, or a combination thereof.

In some embodiments, a polymeric material (e.g., a polymeric coating) has a melting temperature equal to or greater than about 20° C., 30° C., 40° C., 50° C., 60° C., 70° C., 80° C., 90° C., 100° C., 120° C., or equal to or greater than about 150° C.

### Properties of Polymer or Polymeric Materials

A polymeric material of this disclosure formed from the polymerization of a curable resin or polymerizable composition disclosed herein can provide advantageous characteristics compared to conventional polymeric materials. In some instances, a polymeric material can also have low amounts of water uptake and can be solvent resistant. In some cases, a polymeric material can be characterized by one or more of the properties selected from the group consisting of elongation at break, storage modulus, tensile modulus, stress remaining, glass transition temperature, water uptake, hardness, color, transparency, hydrophobicity, lubricity, surface texture, percent crystallinity, phase composition ratio, phase domain size, and phase domain size and morphology. Further, as described herein, the polymeric materials provided herein can be used for a multitude of applications, including 3D printing, to form materials having favorable properties of both elasticity and stiffness. Specifically, a polymeric material of this disclosure can provide excellent flexural modulus, elastic modulus, elongation at break, or a combination thereof.

Some embodiments provide a polymer (e.g., a polymeric coating) formed from the polymerizable composition of any one of the embodiments disclosed herein. In some embodiments, the polymer has one or more of the following characteristics:
(A) a storage modulus greater than or equal to 200 MPa;
(B) a flexural stress and/or flexural modulus of greater than or equal to 1.5 MPa remaining after 24 hours in a wet environment at 37° C.;
(C) an elongation at break greater than or equal to 5% before and after 24 hours in a wet environment at 37° C.;
(D) a water uptake of less than 25 wt % when measured after 24 hours in a wet environment at 37° C.;
(E) transmission of at least 30% of visible light through the polymeric material after 24 hours in a wet environment at 37° C.; and
(F) comprises a plurality of polymeric phases, wherein at least one polymeric phase of the one or more polymeric phases has a T_{g} of at least 60° C., 80° C., 90° C., 100° C., or at least 110° C.

In some embodiments, the polymer (e.g., a polymeric coating) is characterized by a water uptake of less than 20 wt %, less than 15 wt %, less than 10 wt %, less than 5 wt %, less than 4 wt %, less than 3 wt %, less than 2 wt %, less than 1 wt %, less than 0.5 wt %, less than 0.25 wt %, or less than 0.1 wt % when measured after 24 hours in a wet environment at 37° C.

In some embodiments, the polymer has greater than 30%, 40%, 50%, 60%, 80%, 90% or 99% conversion of double bonds to single bonds compared to the polymerizable composition, as measured by FTIR.

In certain embodiments, the polymer has an ultimate tensile strength from 10 MPa to 100 MPa, from 15 MPa to 80 MPa, from 20 MPa to 60 MPa, from 10 MPa to 50 MPa, from 10 MPa to 45 MPa, from 25 MPa to 40 MPa, from 30 MPa to 45 MPa, or from 30 MPa to 40 MPa after 24 hours in a wet environment at 37° C.

In some embodiments, the polymer is characterized by an elongation at break greater than 10%, an elongation at break greater than 20%, an elongation at break greater than 30%, an elongation at break of 5% to 250%, an elongation at break of 20% to 250%, or an elongation at break value from 40% to 250% before and after 24 hours in a wet environment at 37° C.

In certain embodiments, the polymer is characterized by a storage modulus of 0.1 MPa to 4000 MPa, a storage modulus of 300 MPa to 3000 MPa, or a storage modulus of 750 MPa to 3000 MPa after 24 hours in a wet environment at 37° C.

In some embodiments, the polymer has a flexural stress, a flexural modulus, or a flexural stress and flexural modulus of 400 MPa or more, 300 MPa or more, 200 MPa or more, 180 MPa or more, 160 MPa or more, 120 MPa or more, 100 MPa or more, 80 MPa or more, 70 MPa or more, 60 MPa or more, after 24 hours in a wet environment at 37° C.

In certain embodiments, at least 40%, 50%, 60%, or 70% of visible light passes through the polymer after 24 hours in a wet environment at 37° C. In some embodiments, the polymer is biocompatible, bioinert, or a combination thereof.

One embodiment provides a polymeric film comprising a polymer of any one of the embodiments disclosed herein. In some embodiments, the polymeric film has a thickness of at least 100 μm and not more than 3 or 4 mm. In some embodiments, the polymeric film has a thickness of at least 20, 40, 60, 80, 100, 120, 140, 160, 180, 200 μm and not more than 0.5, 1, 2, 3, 3.5, 4, 4.5, 5, 5.5, 6, or 7 mm.

One embodiment provides an orthodontic appliance comprising the polymer of any one of the embodiments disclosed herein or the polymeric film of any one of the embodiments herein. In some embodiments, the orthodontic appliance is a dental appliance (e.g., a dental aligner, a dental expander, or a dental spacer).

Some embodiments provide a device comprising the polymer of any of the embodiments disclosed herein. In some embodiments, the device is a dental appliance. In some embodiments, the device is a dental aligner, a dental expander, or a dental spacer.

In various embodiments, a polymeric material herein can comprise or consist of a high toughness, e.g., through a tough polymer matrix, and the difference (or delta) between the elastic modulus measured at different strain rates (e.g., at 1.7 mm/min and 510 mm/min) can be low, e.g., lower than 80%, 70%, 60%, 50%, 40%, or lower than 30%, which can be an indication for a polymeric phase separation within the material.

In some embodiments, a polymeric material of the present disclosure can have one or more of the following characteristics: (A) a flexural modulus greater than or equal to 50 MPa, 100 MPa, or 200 MPa; (B) an elastic modulus of greater than or equal to 150 MPa, 250 MPa, 350 MPa, 450 MPa, 550 MPa, or from about 500 to 1500 MPa, from about 550 to about 1000 MPa, or from about 550 MPa to about 1500 MPa) an elongation at break greater than or equal to 5% before and after 24 hours in a wet environment at 37° C.; (D) a water uptake of less than 25 wt % when measured after 24 hours in a wet environment at 37° C.; (E) transmission of at least 30% of visible light through the polymeric material after 24 hours in a wet environment at 37° C.; and (F) comprises a plurality of polymeric phases, wherein at least one polymeric phase of the one or more polymeric phases has a Ts of at least 60° C., 80° C., 90° C., 100° C., or at least 110° C. In some instances, a polymeric material herein has at least two, three, four, five, or all characteristics of (A), (B), (C), (D), (E) and (F).

In some instances, the polymeric material can be characterized by a storage modulus of 0.1 MPa to 4000 MPa, a storage modulus of 300 MPa to 3000 MPa, or a storage modulus of 750 MPa to 3000 MPa after 24 hours in a wet environment at 37° C.

In some instances, the polymeric material herein can have a flexural stress remaining of 400 MPa or more, 300 MPa or more, 200 MPa or more, 180 MPa or more, 160 MPa or more, 120 MPa or more, 100 MPa or more, 80 MPa or more, 70 MPa or more, 60 MPa or more, after 24 hours in a wet environment at 37° C.

In some instances, the polymeric material can be characterized by an elongation at break greater than 10%, an elongation at break greater than 20%, an elongation at break greater than 30%, an elongation at break of 5% to 250%, an elongation at break of 20% to 250%, or an elongation at break value from 40% to 250% before and after 24 hours in a wet environment at 37° C.

A polymeric material can be characterized by a water uptake of less than 20 wt %, less than 15 wt %, less than 10 wt %, less than 5 wt %, less than 4 wt %, less than 3 wt %, less than 2 wt %, less than 1 wt %, less than 0.5 wt %, less than 0.25 wt %, or less than 0.1 wt % when measured after 24 hours in a wet environment at 37° C. In some cases, a polymeric material can have greater than 50%, 60%, or 70% conversion of double bonds to single bonds compared to the polymerizable composition, as measured by FTIR.

In some instances, a polymeric material can have an ultimate tensile strength from 10 MPa to 100 MPa, from 15 MPa to 80 MPa, from 20 MPa to 60 MPa, from 10 MPa to 50 MPa, from 10 MPa to 45 MPa, from 25 MPa to 40 MPa, from 30 MPa to 45 MPa, or from 30 MPa to 40 MPa after 24 hours in a wet environment at 37° C.

In some instances, a polymeric material can have a low amount of hydrogen bonding which can facilitate a decreased uptake of water in comparison with conventional polymeric materials having greater amounts of hydrogen bonding. Thus, in some instances, a polymeric material herein can comprise less than about 10 wt %, less than about 9 wt %, less than about 8 wt %, less than about 7 wt %, less than about 6 wt %, less than about 5 wt %, less than about 4 wt %, less than about 3 wt %, less than about 2 wt %, less than about 1 wt %, or less than about 0.5 wt % water when fully saturated at use temperature (e.g., about 20° C., 25° C., 30° C., or 35° C.). In some instances, the use temperature can include the temperature of a human mouth (e.g., approximately 35-40° C.). The use temperature can be a temperature selected from −100-250° C., 0-90° C., 0-80° C., 0-70° C., 0-60° C., 0-50° C., 0-40° C., 0-30° C., 0-20° C., 0-10° C., 20-90° C., 20-80° C., 20-70° C., 20-60° C., 20-50° C., 20-40° C., 20-30° C., or below 0° C.

In various instances, the one or more amorphous phases of the polymeric material can have a glass transition temperature of at least about 30° C., 40° C., 50° C., 60° C., 70° C., 80° C., 90° C., 100° C., or at least about 110° C.

Further provided herein are polymeric films/coatings comprising a polymeric material of the present disclosure. In some cases, such polymeric film can have a thickness of at least about 50 μm, 100 μm, 250 μm, 500 μm, 1 mm, 2 mm and not more than 3 or 4 mm.

The present disclosure provides devices that comprise a polymeric material (e.g., a polymeric coating) of the present disclosure. As described herein, such polymeric material can comprise, incorporated in its polymeric structure, one or more species of polymerizable compound(s) of this disclosure. In various cases, the device can be a medical device. The medical device can be an orthodontic appliance. The orthodontic appliance can be a dental aligner, a dental expander, or a dental spacer.

### Methods of Use

The present disclosure provides methods for synthesizing the polymerizable compound of the present disclosure, methods of using compositions (e.g., resins and polymeric materials) comprising such compounds, as well as methods for using the compositions in devices such as medical devices. In cases in which photo-polymerization is used to cure a resin, a polymerizable compound of the present disclosure can be used as components in materials applicable many different industries such as transportation (e.g., planes, trains, boats, automobiles, etc.), hobbyist, prototyping, medical, art and design, microfluidics, molds, among others. Such medical devices include, in various embodiments herein, orthodontic appliances.

In some embodiments, the present disclosure provides a method of 3D printing using curable resins or polymerizable compositions. In some embodiments, the method includes preparing a medical device. In some embodiments, the method includes preparing a dental retainer or dental aligner. In some embodiment the method includes preparing and/or curing a 3D printing resin.

### Synthetic Methods

The present disclosure provides synthetic methods for producing the polymerizable compositions and polymeric coatings described herein. In some embodiments, any of such methods can comprise isolating the polymerizable coating with a chemical yield of at least about 25%, 30%, 40%, 50%, 60%, 70%, 80%, 90%, or at least about 95%, and a chemical purity of at least about 90%, 95%, or 99%.

One of skill in the art may appreciate that any suitable coupling chemistry (e.g., addition or substitution chemistry including Diels-Alder, click chemistry, etc.) can be used to couple the terminal monomers (TM) to the chain of interconnected monomers (monomer chain), and subsequently attach the reactive functional groups to the terminal monomers. Alternatively, it can be envisioned that the reactive functional groups can be attached to a terminal monomer, which is subsequently coupled to the chain of interconnected monomers. Additionally, one of skill in the art may recognize that protecting groups may be necessary for the preparation of certain compounds and may be aware of those conditions compatible with a selected protecting group.

### Methods of Forming Polymer or Polymeric Coatings

Further provided herein is a method of polymerizing (e.g., photo-curing) a curable composition (e.g., a photo-curable resin) comprising at least one species of a polymerizable compound described herein and optionally one or more additional components selected from the group consisting of telechelic polymers, telechelic oligomers, polymerizable monomers (e.g., reactive diluents), polymerization initiators, polymerization inhibitors, solvents, fillers, antioxidants, pigments, colorants, surface modifiers, and mixtures thereof, to obtain an optionally cross-linked polymer, the method comprising a step of mixing the curable composition, optionally after heating, with a reactive diluent before inducing polymerization by heating and/or irradiating the composition; wherein the reactive diluent is selected from the polymerizable monomers and mixtures thereof.

The present disclosure provides methods for producing polymeric materials (e.g., a polymeric coating) using curable resins or polymerizable compositions described herein. In various embodiments, provided herein are methods for photo-curing a polymerizable composition. Hence, in various instances, provided herein is a method of forming a polymeric material, the method comprising: (i) providing a polymerizable composition of the present disclosure; (ii) exposing the polymerizable composition to a light source; and curing the polymerizable composition to form the polymeric material.

In some embodiments, the photo-curing comprises a single curing step. In some embodiments, the photo-curing comprises a plurality of curing steps. In yet other embodiments, the photo-curing comprises at least one curing step which exposes the curable resin or polymerizable composition to light. Exposing the curable resin or polymerizable composition to light can initiate and/or facilitate photo-polymerization. In some instances, a photoinitiator can be used as part of the resin to accelerate and/or initiate photo-polymerization. In some embodiments, the resin is exposed to UV (ultraviolet) light, visible light, IR (infrared) light, or any combination thereof. In some embodiments, the cured polymeric material is formed from the polymerizable composition using at least one step comprising exposure to a light source, wherein the light source comprises UV light, visible light, and/or IR light. In some embodiments, the light source comprises a wavelength from 10 nm to 200 nm, from 200 nm to 350 nm, from 350 nm to 450 nm, from 450 nm to 550 nm, from 550 nm to 650 nm, from 650 nm to 750 nm, from 750 nm to 850 nm, from 850 nm to 1000 nm, or from 1000 nm to 1500 nm.

In some instances, the polymeric material has the glass transition temperature (T_{g}) of at least about 40° C., 50° C., 60° C., 80° C., 90° C., 100° C., 110° C. or at least about 120° C.

In some embodiments, a method of forming a polymeric material from a polymerizable composition described herein can further comprise initiating and/or enhancing formation of crystalline phases in the forming polymeric material. In certain embodiments, the triggering comprises cooling the cured material, adding seeding particles to the polymerizable composition, providing a force to the cured material, providing an electrical charge to the polymerizable composition, or any combination thereof. In some cases, polymer crystals can yield upon application of a strain (e.g., a physical strain, such as twisting or stretching a material). The yielding may include unraveling, unwinding, disentangling, dislocation, coarse slips, and/or fine slips in the crystallized polymer. In some embodiments, the methods disclosed herein further comprise the step of growing polymer crystals. As described further herein, polymer crystals comprise the crystallizable polymeric material.

Thus, in various embodiments, a method of forming a polymeric material from a polymerizable composition described herein can comprise inducing phase separation in the forming polymeric material (i.e., during photo-curing), wherein such phase separation can yield polymeric materials that comprise one or more amorphous phases, one or more crystalline phases, or both one or more amorphous phases and one or more crystalline phases.

As described herein, a polymeric material produced by the methods provided herein can be characterized by one or more of: (i) a storage modulus greater than or equal to 200 MPa; (ii) a flexural stress of greater than or equal to 1.5 MPa remaining after 24 hours in a wet environment at 37° C.; (iii) an elongation at break greater than or equal to 5% before and after 24 hours in a wet environment at 37° C.; (iv) a water uptake of less than 25 wt % when measured after 24 hours in a wet environment at 37° C.; and (v) transmission of at least 30% of visible light through the polymeric material after 24 hours in a wet environment at 37° C. In various cases, such polymeric material can be characterized by at least 2, 3, 4, or all of these properties.

### Fabrication and Use of Orthodontic Appliances

Provided herein are methods for using the polymerizable compounds, curable resins and compositions comprising such compounds, as well as polymeric materials produced from such resins and composition for the fabrication of a medical device, such as an orthodontic appliance (e.g., a dental aligner, a dental expander, or a dental spacer).

Thus, in some embodiments, a method herein further comprises the step of fabricating a device or an object using an additive manufacturing device, wherein the additive manufacturing device facilitates the curing. In some embodiments, the curing of a polymerizable composition produces the cured polymeric material. In certain embodiments, a polymerizable composition is cured using an additive manufacturing device to produce the cured polymeric material. In some embodiments, the method further comprises the step of cleaning the cured polymeric material. In certain embodiments, the cleaning of the cured polymeric material includes washing and/or rinsing the cured polymeric material with a solvent, which can remove uncured resin and undesired impurities from the cured polymeric material.

In some embodiments, a polymerizable composition herein can be curable and have melting points<100° C. in order to be liquid and, thus, processable at the temperatures usually employed in currently available additive manufacturing techniques. As described herein, the polymerizable monomers of the present disclosure that are used as components in the polymerizable compositions can have a low vapor pressure at an elevated temperature compared to conventional reactive diluents or other polymerizable components used in polymerizable compositions. Such low vapor pressure of the monomers described herein can be particularly advantageous for use of such monomer in the curable (e.g., photocurable) compositions and additive manufacturing where elevated temperatures (e.g., 60° C., 80° C., 90° C., or higher) may be used. In various instances, a polymerizable monomer can have a vapor pressure of at most about 12 Pa at 60° C., or lower, as further described herein.

In some embodiments, a curable resin or polymerizable composition herein can comprise at least one photo-polymerization initiator (i.e., a photoinitiator) and may be heated to a predefined elevated process temperature ranging from about 50° C. to about 120° C., such as from about 90° C. to about 120° C., before becoming irradiated with light of a suitable wavelength to be absorbed by the photoinitiator, thereby causing activation of the photoinitiator to induce polymerization of the curable resin or polymerizable composition to obtain a cured polymeric material, which can optionally be cross-linked.

In some embodiments, the methods disclosed herein for forming a polymeric material are part of a high temperature lithography-based photo-polymerization process, wherein a polymerizable composition that can comprise at least one photo-polymerization initiator is heated to an elevated process temperature (e.g., from about 50° C. to about 120° C., such as from about 90° C. to about 120° C.). Thus, a method for forming a polymeric material according to the present disclosure can offer the possibility of quickly and facilely producing devices, such as orthodontic appliances, by additive manufacturing such as 3D printing using polymerizable compositions as disclosed herein. In various embodiments, such curable resin or polymerizable composition is a photo-curable resin comprising one or more photo-polymerizable compounds described herein.

One embodiment provides a method of forming a polymer (e.g., a second polymer) according to the embodiments disclosed herein, the method comprising:
providing a polymerizable composition according to the embodiments disclosed herein;
exposing the polymerizable composition to a light source; and
polymerizing the polymerizable composition to form the polymer.

In some embodiments, the light source is an ultraviolet (UV) or visible light source. In certain embodiments, the method further comprising fabricating an orthodontic appliance with the polymer.

One embodiment provides a method for preparing an article by an additive manufacturing process, comprising:
providing a polymerizable composition according to the embodiments disclosed herein;
exposing the polymerizable composition to radiation;
polymerizing the polymerizable composition layer-by-layer based on a predefined design, thereby polymerizing the polymerizable composition to form a polymer (e.g., a second polymer); and
fabricating the article with the polymer (e.g., the second polymer).

In some embodiments, the method further comprises heating the polymerizable composition to a processing temperature.

In some embodiments, the processing temperature is from about 50° C. to about 120° C. In certain embodiments, the processing temperature is from about 90° C. to about 110° C., from about 100° C. to about 120° C., from about 105° C. to about 115° C., or from about 108° C. to about 110° C. In some embodiments, the additive manufacturing process is a 3D printing process.

In some embodiments, the method further comprises receiving a file containing instructions for fabrication of a dental appliance.

In some embodiments, the article is a medical device. In certain embodiments, the medical device is an orthodontic appliance.

Further embodiments provide a method of repositioning a patient's teeth, comprising: generating a treatment plan for the patient, the plan comprising a plurality of intermediate tooth arrangements for moving teeth along a treatment path from an initial tooth arrangement toward a final tooth arrangement;
producing an orthodontic appliance according to the embodiments disclosed herein, or an orthodontic appliance comprising the polymer according to the embodiments disclosed herein; and
moving on-track, with the orthodontic appliance, at least one of the patient's teeth toward an intermediate tooth arrangement or the final tooth arrangement.

In some embodiments, producing the orthodontic appliance comprises 3D printing of the orthodontic appliance.

In some embodiments, the method further comprises tracking progression of the patient's teeth along the treatment path after administration of the orthodontic appliance to the patient, the tracking comprising comparing a current arrangement of the patient's teeth to a planned arrangement of the patient's teeth.

In certain embodiments, greater than 60% of the patient's teeth are on track with the treatment plan after 2 weeks of treatment.

In some embodiments, the orthodontic appliance has a retained repositioning force to the at least one of the patient's teeth after 2 days that is at least 10%, at least 20%, at least 30%, at least 40%, at least 50%, at least 60%, or at least 70% of repositioning force initially provided to the at least one of the patient's teeth.

In some embodiments, the treatment plan is generated using an intraoral scan of the patient's teeth.

Photo-polymerization can occur when a polymerizable composition herein is exposed to radiation (e.g., UV or visible light) of a wavelength sufficient to initiate polymerization. The wavelengths of radiation useful to initiate polymerization may depend on the photoinitiator used. “Light” as used herein includes any wavelength and power capable of initiating polymerization. Some wavelengths of light include ultraviolet (UV) or visible. UV light sources include UVA (wavelength about 400 nanometers (nm) to about 320 nm), UVB (about 320 nm to about 290 nm) or UVC (about 290 nm to about 100 nm). Any suitable source may be used, including laser sources. The source may be broadband or narrowband, or a combination thereof. The light source may provide continuous or pulsed light during the process. Both the length of time the system is exposed to UV light and the intensity of the UV light can be varied to determine the ideal reaction conditions.

In some embodiments, the methods disclosed herein include the use of additive manufacturing to produce a device comprising the cured polymeric material. Such device can be an orthodontic appliance. The orthodontic appliance can be a dental aligner, a dental expander, or a dental spacer. In certain embodiments, the methods disclosed herein use additive manufacturing to produce a device comprising, consisting essentially of, or consisting of the cured polymeric material. Additive manufacturing includes a variety of technologies which fabricate three-dimensional objects directly from digital models through an additive process. In some embodiments, successive layers of material are deposited and “cured in place”. A variety of techniques are known to the art for additive manufacturing, including selective laser sintering (SLS), fused deposition modeling (FDM) and jetting or extrusion. In many embodiments, selective laser sintering involves using a laser beam to selectively melt and fuse a layer of powdered material according to a desired cross-sectional shape to build up the object geometry. In many embodiments, fused deposition modeling involves melting and selectively depositing a thin filament of thermoplastic polymer in a layer-by-layer manner to form an object. In yet another example, 3D printing can be used to fabricate an orthodontic appliance herein. In many embodiments, 3D printing involves jetting or extruding one or more materials (e.g., the crystallizable resins disclosed herein) onto a build surface to form successive layers of the object geometry. In some embodiments, a polymerizable composition described herein can be used in inkjet or coating applications. Cured polymeric materials may also be fabricated by “vat” processes in which light is used to selectively cure a vat or reservoir of the curable resin or polymerizable composition. Each layer of curable resin or polymerizable composition may be selectively exposed to light in a single exposure or by scanning a beam of light across the layer. Specific techniques that can be used herein can include stereolithography (SLA), Digital Light Processing (DLP) and two photon-induced photo-polymerization (TPIP).

In some embodiments, the methods disclosed herein use continuous direct fabrication to produce a device comprising the cured polymeric material. Such device can be an orthodontic appliance as described herein. In certain embodiments, the methods disclosed herein can comprise the use of continuous direct fabrication to produce a device (e.g., an orthodontic appliance) comprising, consisting essentially of, or consisting of the cured polymeric material. A non-limiting exemplary direct fabrication process can achieve continuous build-up of an object geometry by continuous movement of a build platform (e.g., along the vertical or Z-direction) during an irradiation phase, such that the hardening depth of the irradiated photo-polymer (e.g., an irradiated polymerizable composition, hardening during the formation of a cured polymeric material) is controlled by the movement speed. Accordingly, continuous polymerization of material (e.g., polymerization of a polymerizable composition into a cured polymeric material) on the build surface can be achieved. Such methods are described in U.S. Pat. No. 7,892,474, the disclosure of which is incorporated herein by reference in its entirety. In yet another example, a continuous direct fabrication method utilizes a “heliolithography” approach in which a liquid resin (e.g., a polymerizable composition) is cured with focused radiation while the build platform is continuously rotated and raised. Accordingly, the object geometry can be continuously built up along a spiral build path. Such methods are described in U.S. Patent Publication No. 2014/0265034, the disclosure of which is incorporated herein by reference in its entirety. Continuous liquid interface production of 3D objects has also been reported (J. Tumbleston et al., Science, 2015, 347 (6228), pp 1349-1352), which reference is hereby incorporated by reference in its entirety for description of the process. Another example of continuous direct fabrication method can involve extruding a material composed of a curable liquid material or resin surrounding a solid strand. The material can be extruded along a continuous three-dimensional path to form the object. Such methods are described in U.S. Patent Publication No. 2014/0061974, the disclosure of which is incorporated herein by reference in its entirety.

In some embodiments, the methods disclosed herein can comprise the use of high temperature lithography to produce a device comprising the cured polymeric material. Such device can be an orthodontic appliance as described herein. In certain embodiments, the methods disclosed herein use high temperature lithography to produce a device comprising, consisting essentially of, or consisting of the cured polymeric material. “High temperature lithography,” as used herein, may refer to any lithography-based photo-polymerization processes that involve heating photo-polymerizable material(s) (e.g., a polymerizable composition disclosed herein). The heating may lower the viscosity of the polymerizable composition before and/or during curing. Non-limiting examples of high-temperature lithography processes include those processes described in WO 2015/075094, WO 2016/078838 and WO 2018/032022. In some implementations, high-temperature lithography may involve applying heat to material to temperatures from about 50° C. to about 120° C., such as from about 90° C. to about 120° C., from about 100° C. to about 120° C., from about 105° C. to about 115° C., from about 108° C. to about 110° C., etc. The material may be heated to temperatures greater than about 120° C. It is noted other temperature ranges may be used without departing from the scope and substance of the inventive concepts described herein.

Since, in some cases, the polymerizable compounds of the present disclosure can, as part of a polymerizable composition, become co-polymerized in the polymerization process of a method according to the present disclosure, the result can be an optionally cross-linked polymer comprising moieties of one or more species of polymerizable compound(s) as repeating units. In some cases, such polymer is a cross-linked polymer which, typically, can be suitable and useful for applications in orthodontic appliances. The polymerizable compounds of this disclosure comprising a plurality of reactive functional groups can provide uniform and continuous polymeric networks with clear phase separation.

In further embodiments, a method herein can comprise polymerizing a curable composition which comprises at least one polymerizable compound, which, upon polymerization, can furnish a cross-linked polymer matrix which can comprise moieties originating from the polymerizable compound(s) of the present disclosure as repeating units. To obtain cross-linked polymers which can be particularly suitable as orthodontic appliances, the at least one polymerizable species used in the method according to the present disclosure can be selected regarding several thermomechanical properties of the resulting polymers. In some instances, a curable resin or polymerizable composition of the present disclosure can comprise one or more species of polymerizable compounds. In some cases, a polymerizable monomer of the present disclosure can also have cross-linking functionalities, in instances where it contains a plurality of reactive functional groups (like the polymerizable compounds herein), and thus not only act as a reactive diluent with low vapor pressure, but also as a cross-linking agent during polymerization of a curable resin or polymerizable composition described herein. In other embodiments, a resin comprises a polymerizable compound as described herein, a polymerizable monomer, and a cross-linking monomer, wherein both monomers are different species (i.e., chemical entities).

### Orthodontic Appliances and Uses Thereof

The polymerizable compounds according to the present disclosure can be used as components for viscous or highly viscous polymerizable compositions and can result in polymeric materials that can have favorable thermomechanical properties as described herein (e.g., stiffness, stress remaining, etc.) for use in orthodontic appliances, for example, for moving one or more teeth of a patient.

As described herein, the present disclosure provides a method of repositioning a patient's teeth, the method comprising: (i) generating a treatment plan for the patient, the plan comprising a plurality of intermediate tooth arrangements for moving teeth along a treatment path from an initial tooth arrangement toward a final tooth arrangement; (ii) producing a dental appliance comprising a polymeric material described herein; and moving on-track, with the dental appliance, at least one of the patient's teeth toward an intermediate tooth arrangement or the final tooth arrangement. Such dental appliance can be produced using processes that include 3D printing, as further described herein. The method of repositioning a patient's teeth can further comprise tracking progression of the patient's teeth along the treatment path after administration of the dental appliance to the patient, the tracking comprising comparing a current arrangement of the patient's teeth to a planned arrangement of the patient's teeth. In such instances, greater than 60% of the patient's teeth can be on track with the treatment plan after 2 weeks of treatment. In some instances, the dental appliance has a retained repositioning force to the at least one of the patient's teeth after 2 days that is at least 10%, at least 20%, at least 30%, at least 40%, at least 50%, at least 60%, or at least 70% of repositioning force initially provided to the at least one of the patient's teeth.

As used herein, the terms “rigidity” and “stiffness” can be used interchangeably, as are the corresponding terms “rigid” and “stiff.” As used herein a “plurality of teeth” encompasses two or more teeth.

In many embodiments, one or more posterior teeth comprises one or more of a molar, a premolar, or a canine, and one or more anterior teeth comprising one or more of a central incisor, a lateral incisor, a cuspid, a first bicuspid or a second bicuspid.

In some embodiments, the compositions and methods described herein can be used to couple groups of one or more teeth to each other. The groups of one or more teeth may comprise a first group of one or more anterior teeth and a second group of one or more posterior teeth. The first group of teeth can be coupled to the second group of teeth with the polymeric shell appliances as disclosed herein.

The embodiments disclosed herein are well suited for moving one or more teeth of the first group of one or more teeth or moving one or more of the second group of one or more teeth, and combinations thereof.

The embodiments disclosed herein are well suited for combination with one or more known commercially available tooth moving components such as attachments and polymeric shell appliances. In many embodiments, the appliance and one or more attachments are configured to move one or more teeth along a tooth movement vector comprising six degrees of freedom, in which three degrees of freedom are rotational and three degrees of freedom are translation.

The present disclosure provides orthodontic systems and related methods for designing and providing improved or more effective tooth moving systems for eliciting a desired tooth movement and/or repositioning teeth into a desired arrangement.

Although reference is made to an appliance comprising a polymeric shell appliance, the embodiments disclosed herein are well suited for use with many appliances that receive teeth, for example appliances without one or more of polymers or shells. The appliance can be fabricated with one or more of many materials such as metal, glass, reinforced fibers, carbon fiber, composites, reinforced composites, aluminum, biological materials, and combinations thereof, for example. In some cases, the reinforced composites can comprise a polymer matrix reinforced with ceramic or metallic particles, for example. The appliance can be shaped in many ways, such as with thermoforming or direct fabrication as described herein, for example. Alternatively, or in combination, the appliance can be fabricated with machining such as an appliance fabricated from a block of material with computer numeric control machining. In some cases, the appliance is fabricated using a polymerizable compound according to the present disclosure, for example, using the monomers as reactive diluents for curable resins or polymerizable compositions.

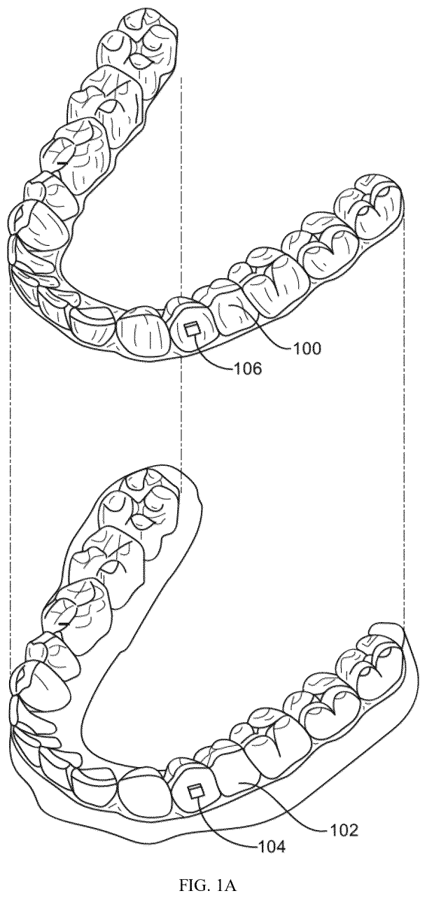

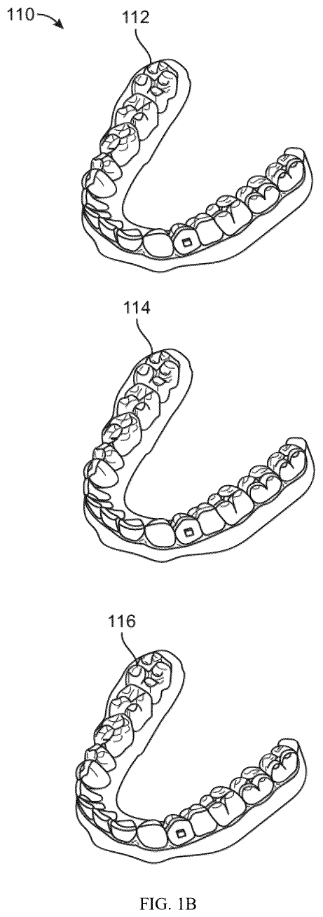

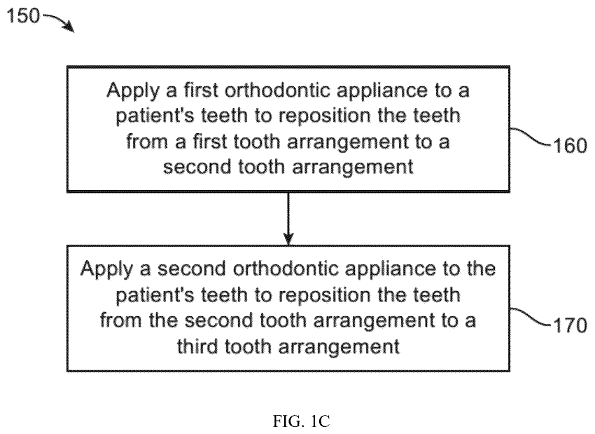

Turning now to the drawings, in which like numbers designate like elements in the various figures, FIG. 1A illustrates an exemplary tooth repositioning appliance or aligner 100 that can be worn by a patient to achieve an incremental repositioning of individual teeth 102 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using rapid prototyping fabrication techniques, from a digital model of an appliance. An appliance can fit over all teeth present in an upper or lower jaw, or less than all the teeth. The appliance can be designed specifically to accommodate the teeth of the patient (e.g., the topography of the tooth-receiving cavities matches the topography of the patient's teeth) and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth, or teeth targeted for repositioning. In some cases, some, most, or even all the teeth will be repositioned at some point during treatment. Teeth that are moved can also serve as a base or anchor for holding the appliance as it is worn by the patient. Typically, no wires or other means will be provided for holding an appliance in place over the teeth. In some cases, however, it may be desirable or necessary to provide individual attachments or other anchoring elements 104 on teeth 102 with corresponding receptacles or apertures 106 in the appliance 100 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the url “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 1B illustrates a tooth repositioning system 110 including a plurality of appliances 112, 114, 116. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 110 can include a first appliance 112 corresponding to an initial tooth arrangement, one or more intermediate appliances 114 corresponding to one or more intermediate arrangements, and a final appliance 116 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

FIG. 1C illustrates a method 150 of orthodontic treatment using a plurality of appliances, in accordance with embodiments. The method 150 can be practiced using any of the appliances or appliance sets described herein. In step 160, a first orthodontic appliance is applied to a patient's teeth to reposition the teeth from a first tooth arrangement to a second tooth arrangement. In step 170, a second orthodontic appliance is applied to the patient's teeth to reposition the teeth from the second tooth arrangement to a third tooth arrangement. The method 150 can be repeated as necessary using any suitable number and combination of sequential appliances to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied to compensate for any inaccuracies or limitations of the appliance.

The various embodiments of the orthodontic appliances presented herein can be fabricated in a wide variety of ways. In some embodiments, the orthodontic appliances herein (or portions thereof) can be produced using direct fabrication, such as additive manufacturing techniques (also referred to herein as “3D printing”) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can be categorized as follows: (1) vat photo-polymerization (e.g., stereolithography), in which an object is constructed layer by layer from a vat of liquid photo-polymer resin/polymerizable composition; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances herein. In some embodiments, stereolithography involves selective polymerization of a photosensitive resin (e.g., a photo-polymer or polymerizable composition) according to a desired cross-sectional shape using light (e.g., ultraviolet light). The object geometry can be built up in a layer-by-layer fashion by sequentially polymerizing a plurality of object cross-sections. As another example, the appliances herein can be directly fabricated using selective laser sintering. In some embodiments, selective laser sintering involves using a laser beam to selectively melt and fuse a layer of powdered material according to a desired cross-sectional shape to build up the object geometry. Yet another example, the appliances herein can be directly fabricated by fused deposition modeling. In some embodiments, fused deposition modeling involves melting and selectively depositing a thin filament of thermoplastic polymer in a layer-by-layer manner to form an object. In yet another example, material jetting can be used to directly fabricate the appliances herein. In some embodiments, material jetting involves jetting or extruding one or more materials onto a build surface to form successive layers of the object geometry.

Alternatively, or in combination, some embodiments of the appliances herein (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold to generate an appliance shell.

In some embodiments, the direct fabrication methods provided herein build up the object geometry in a layer-by-layer fashion, with successive layers being formed in discrete build steps. Alternatively, or in combination, direct fabrication methods that allow for continuous build-up of an object geometry can be used, referred to herein as “continuous direct fabrication.” Various types of continuous direct fabrication methods can be used. As an example, in some embodiments, the appliances herein are fabricated using “continuous liquid interphase printing,” in which an object is continuously built up from a reservoir of photo-polymerizable resin by forming a gradient of partially cured resin between the building surface of the object and a polymerization-inhibited “dead zone.” In some embodiments, a semi-permeable membrane is used to control transport of a photo-polymerization inhibitor (e.g., oxygen) into the dead zone to form the polymerization gradient. Continuous liquid interphase printing can achieve fabrication speeds about 25 times to about 100 times faster than other direct fabrication methods, and speeds about 1000 times faster can be achieved with the incorporation of cooling systems. Continuous liquid interphase printing is described in U.S. Patent Publication Nos. 2015/0097315, 2015/0097316, and 2015/0102532, the disclosures of each of which are incorporated herein by reference in their entirety.

As another example, a continuous direct fabrication method can achieve continuous build-up of an object geometry by continuous movement of the build platform (e.g., along the vertical or Z-direction) during the irradiation phase, such that the hardening depth of the irradiated photo-polymer is controlled by the movement speed. Accordingly, continuous polymerization of material on the build surface can be achieved. Such methods are described in U.S. Pat. No. 7,892,474, the disclosure of which is incorporated herein by reference in its entirety.

In another example, a continuous direct fabrication method can involve extruding a composite material composed of a curable liquid material surrounding a solid strand. The composite material can be extruded along a continuous three-dimensional path to form the object. Such methods are described in U.S. Patent Publication No. 2014/0061974, the disclosure of which is incorporated herein by reference in its entirety.

In yet another example, a continuous direct fabrication method utilizes a “heliolithography” approach in which the liquid photo-polymer is cured with focused radiation while the build platform is continuously rotated and raised. Accordingly, the object geometry can be continuously built up along a spiral build path. Such methods are described in U.S. Patent Publication No. 2014/0265034, the disclosure of which is incorporated herein by reference in its entirety.

The direct fabrication approaches provided herein are compatible with a wide variety of materials, including but not limited to one or more of the following: a polyester, a co-polyester, a polycarbonate, a thermoplastic polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, a polytrimethylene terephthalate, a styrenic block copolymer (SBC), a silicone rubber, an elastomeric alloy, a thermoplastic elastomer (TPE), a thermoplastic vulcanizate (TPV) elastomer, a polyurethane elastomer, a block copolymer elastomer, a polyolefin blend elastomer, a thermoplastic co-polyester elastomer, a thermoplastic polyamide elastomer, a thermoset material, or combinations thereof. The materials used for direct fabrication can be provided in an uncured form (e.g., as a liquid, resin, powder, etc.) and can be cured (e.g., by photo-polymerization, light curing, gas curing, laser curing, cross-linking, etc.) to form an orthodontic appliance or a portion thereof. The properties of the material before curing may differ from the properties of the material after curing. Once cured, the materials herein can exhibit sufficient strength, stiffness, durability, biocompatibility, etc. for use in an orthodontic appliance. The post-curing properties of the materials used can be selected according to the desired properties for the corresponding portions of the appliance.

In some embodiments, relatively rigid portions of the orthodontic appliance can be formed via direct fabrication using one or more of the following materials: a polyester, a co-polyester, a polycarbonate, a thermoplastic polyurethane, a polypropylene, a polyethylene, a polypropylene and polyethylene copolymer, an acrylic, a cyclic block copolymer, a polyetheretherketone, a polyamide, a polyethylene terephthalate, a polybutylene terephthalate, a polyetherimide, a polyethersulfone, and/or a polytrimethylene terephthalate.

In some embodiments, relatively elastic portions of the orthodontic appliance can be formed via direct fabrication using one or more of the following materials: a styrenic block copolymer (SBC), a silicone rubber, an elastomeric alloy, a thermoplastic elastomer (TPE), a thermoplastic vulcanizate (TPV) elastomer, a polyurethane elastomer, a block copolymer elastomer, a polyolefin blend elastomer, a thermoplastic co-polyester elastomer, and/or a thermoplastic polyamide elastomer.

Machine parameters can include curing parameters. For digital light processing (DLP)-based curing systems, curing parameters can include power, curing time, and/or grayscale of the full image. For laser-based curing systems, curing parameters can include power, speed, beam size, beam shape and/or power distribution of the beam. For printing systems, curing parameters can include material drop size, viscosity, and/or curing power. These machine parameters can be monitored and adjusted on a regular basis (e.g., some parameters at every 1-x layers and some parameters after each build) as part of the process control on the fabrication machine. Process control can be achieved by including a sensor on the machine that measures power and other beam parameters every layer or every few seconds and automatically adjusts them with a feedback loop. For DLP machines, gray scale can be measured and calibrated before, during, and/or at the end of each build, and/or at predetermined time intervals (e.g., every nm build, once per hour, once per day, once per week, etc.), depending on the stability of the system. In addition, material properties and/or photo-characteristics can be provided to the fabrication machine, and a machine process control module can use these parameters to adjust machine parameters (e.g., power, time, gray scale, etc.) to compensate for variability in material properties. By implementing process controls for the fabrication machine, reduced variability in appliance accuracy and residual stress can be achieved.

Optionally, the direct fabrication methods described herein allow for fabrication of an appliance including multiple materials, referred to herein as “multi-material direct fabrication.” In some embodiments, a multi-material direct fabrication method involves concurrently forming an object from multiple materials in a single manufacturing step. For instance, a multi-tip extrusion apparatus can be used to selectively dispense multiple types of materials from distinct material supply sources to fabricate an object from a plurality of different materials. Such methods are described in U.S. Pat. No. 6,749,414, the disclosure of which is incorporated herein by reference in its entirety. Alternatively, or in combination, a multi-material direct fabrication method can involve forming an object from multiple materials in a plurality of sequential manufacturing steps. For instance, a first portion of the object can be formed from a first material in accordance with any of the direct fabrication methods herein, then a second portion of the object can be formed from a second material in accordance with methods herein, and so on, until the entirety of the object has been formed.

Direct fabrication can provide various advantages compared to other manufacturing approaches. For instance, in contrast to indirect fabrication, direct fabrication permits production of an orthodontic appliance without utilizing any molds or templates for shaping the appliance, thus reducing the number of manufacturing steps involved and improving the resolution and accuracy of the final appliance geometry. Additionally, direct fabrication permits precise control over the three-dimensional geometry of the appliance, such as the appliance thickness. Complex structures and/or auxiliary components can be formed integrally as a single piece with the appliance shell in a single manufacturing step, rather than being added to the shell in a separate manufacturing step. In some embodiments, direct fabrication is used to produce appliance geometries that would be difficult to create using alternative manufacturing techniques, such as appliances with very small or fine features, complex geometric shapes, undercuts, interproximal structures, shells with variable thicknesses, and/or internal structures (e.g., for improving strength with reduced weight and material usage). For example, in some embodiments, the direct fabrication approaches herein permit fabrication of an orthodontic appliance with feature sizes of less than or equal to about 5 μm, or within a range from about 5 μm to about 50 μm, or within a range from about 20 μm to about 50 μm.

The direct fabrication techniques described herein can be used to produce appliances with substantially isotropic material properties, e.g., substantially the same or similar strengths along all directions. In some embodiments, the direct fabrication approaches herein permit production of an orthodontic appliance with a strength that varies by no more than about 25%, about 20%, about 15%, about 10%, about 5%, about 1%, or about 0.5% along all directions. Additionally, the direct fabrication approaches herein can be used to produce orthodontic appliances at a faster speed compared to other manufacturing techniques. In some embodiments, the direct fabrication approaches herein allow for production of an orthodontic appliance in a time interval less than or equal to about 1 hour, about 30 minutes, about 25 minutes, about 20 minutes, about 15 minutes, about 10 minutes, about 5 minutes, about 4 minutes, about 3 minutes, about 2 minutes, about 1 minutes, or about 30 seconds. Such manufacturing speeds allow for rapid “chair-side” production of customized appliances, e.g., during a routine appointment or checkup.

In some embodiments, the direct fabrication methods described herein implement process controls for various machine parameters of a direct fabrication system or device in order to ensure that the resultant appliances are fabricated with a high degree of precision. Such precision can be beneficial for ensuring accurate delivery of a desired force system to the teeth in order to effectively elicit tooth movements. Process controls can be implemented to account for process variability arising from multiple sources, such as the material properties, machine parameters, environmental variables, and/or post-processing parameters.

Material properties may vary depending on the properties of raw materials, purity of raw materials, and/or process variables during mixing of the raw materials. In many embodiments, resins or other materials for direct fabrication should be manufactured with tight process control to ensure little variability in photo-characteristics, material properties (e.g., viscosity, surface tension), physical properties (e.g., modulus, strength, elongation) and/or thermal properties (e.g., glass transition temperature, heat deflection temperature). Process control for a material manufacturing process can be achieved with screening of raw materials for physical properties and/or control of temperature, humidity, and/or other process parameters during the mixing process. By implementing process controls for the material manufacturing procedure, reduced variability of process parameters and more uniform material properties for each batch of material can be achieved. Residual variability in material properties can be compensated with process control on the machine, as discussed further herein.

Machine parameters can include curing parameters. For digital light processing (DLP)-based curing systems, curing parameters can include power, curing time, and/or grayscale of the full image. For laser-based curing systems, curing parameters can include power, speed, beam size, beam shape and/or power distribution of the beam. For printing systems, curing parameters can include material drop size, viscosity, and/or curing power. These machine parameters can be monitored and adjusted on a regular basis (e.g., some parameters at every 1-x layers and some parameters after each build) as part of the process control on the fabrication machine. Process control can be achieved by including a sensor on the machine that measures power and other beam parameters every layer or every few seconds and automatically adjusts them with a feedback loop. For DLP machines, gray scale can be measured and calibrated at the end of each build. In addition, material properties and/or photo-characteristics can be provided to the fabrication machine, and a machine process control module can use these parameters to adjust machine parameters (e.g., power, time, gray scale, etc.) to compensate for variability in material properties. By implementing process controls for the fabrication machine, reduced variability in appliance accuracy and residual stress can be achieved.

In many embodiments, environmental variables (e.g., temperature, humidity, Sunlight or exposure to other energy/curing source) are maintained in a tight range to reduce variability in appliance thickness and/or other properties. Optionally, machine parameters can be adjusted to compensate for environmental variables.

In many embodiments, post-processing of appliances includes cleaning, post-curing, and/or support removal processes. Relevant post-processing parameters can include purity of cleaning agent, cleaning pressure and/or temperature, cleaning time, post-curing energy and/or time, and/or consistency of support removal process. These parameters can be measured and adjusted as part of a process control scheme. In addition, appliance physical properties can be varied by modifying the post-processing parameters. Adjusting post-processing machine parameters can provide another way to compensate for variability in material properties and/or machine properties.

The configuration of the orthodontic appliances herein can be determined according to a treatment plan for a patient, e.g., a treatment plan involving successive administration of a plurality of appliances for incrementally repositioning teeth. Computer-based treatment planning and/or appliance manufacturing methods can be used in order to facilitate the design and fabrication of appliances. For instance, one or more of the appliance components described herein can be digitally designed and fabricated with the aid of computer-controlled manufacturing devices (e.g., computer numerical control (CNC) milling, computer-controlled rapid prototyping such as 3D printing, etc.). The computer-based methods presented herein can improve the accuracy, flexibility, and convenience of appliance fabrication.

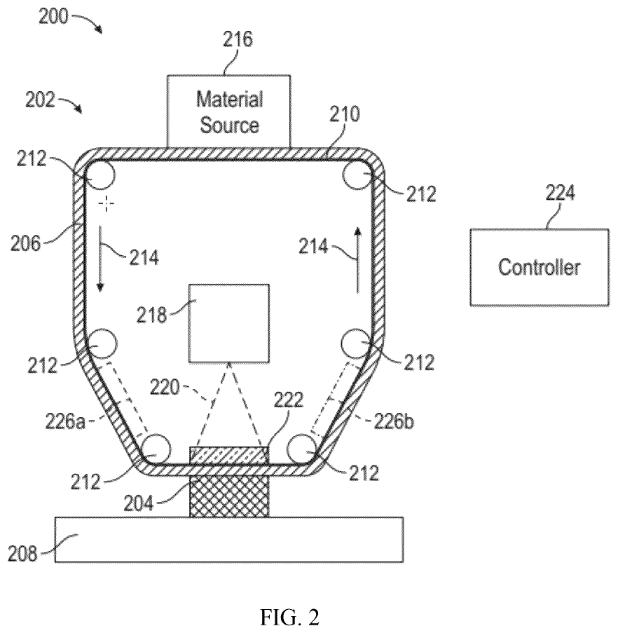

FIG. 2 illustrates a representative example of a system 200 for additive manufacturing configured in accordance with embodiments of the present technology. The system 200 can be used to fabricate any embodiment of the objects described herein. For example, the system 200 can be used to produce an object using an additive manufacturing process (e.g., 3D printing).

The system 200 includes a printer assembly 202 configured to fabricate an additively manufactured object 204 (“object 204”) using any of the additive manufacturing processes described herein. The printer assembly 202 is configured to deposit a curable material 206 (e.g., a polymeric resin, polymerizable composition, or other solidifiable precursor material) on a build platform 208 (e.g., a tray, plate, film, sheet, or other planar substrate) to form the object 204. In the illustrated embodiment, the printer assembly 202 includes a carrier film 210 configured to deliver the curable material 206 to the build platform 208. The carrier film 210 can be a flexible loop of material having an outer surface and an inner surface. The outer surface of the carrier film 210 can adhere to and carry a thin layer of the curable material 206. The inner surface of the carrier film 210 can contact one or more rollers 212 that rotate to move the carrier film 210 in a continuous loop trajectory, e.g., along the direction indicated by arrow 214.

The printer assembly 202 can also include a material source 216 (shown schematically) configured to apply the curable material 206 to the carrier film 210. In the illustrated embodiment, the material source 216 is located at the upper portion of the printer assembly 202. In other embodiments, however, the material source 216 can be at a different location in the printer assembly 202. The material source 216 can include nozzles, ports, reservoirs, etc., that deposit the curable material 206 onto the outer surface of the carrier film 210. The material source 216 can also include one or more blades (e.g., doctor blades, recoater blades) that smooth the deposited curable material 206 into a relatively thin, uniform layer. For example, the curable material 206 can be formed into a layer having a thickness within a range from 200 microns to 300 microns, or any other desired thickness.

The curable material 206 can be conveyed by the carrier film 210 toward the build platform 208. In the illustrated embodiment, the build platform 208 is located below the printer assembly 202. In other embodiments, however, the build platform 208 can be positioned at a different location in the printer assembly 202. The distance between the carrier film 210 and build platform 208 can be adjustable so that the curable material 206 at can be brought into direct contact with the surface of the build platform 208 (when printing the initial layer of the object 204) or with the surface of the object 204 (when printing subsequent layers of the object 204). For example, the build platform 208 can include or be coupled to a motor (not shown) that raises and/or lowers the build platform 208 to the desired height during the manufacturing process.

The printer assembly 202 includes an energy source 218 (e.g., a projector or light engine) that outputs energy 220 (e.g., light, such as UV light) having a wavelength configured to partially or fully cure the curable material 206. The carrier film 210 can be partially or completely transparent to the wavelength of the energy 220 to allow the energy 220 to pass through the carrier film 210 and onto the portion of the curable material 206 above the build platform 208. Optionally, a transparent plate 222 can be disposed between the energy source 218 and the carrier film 210 to guide the carrier film 210 into a specific position (e.g., height) relative to the build platform 208. During operation, the energy 220 can be patterned or scanned in a suitable pattern onto the curable material 206, thus forming a layer of cured material onto the build platform 208 and/or on a previously formed portion of the object 204. The geometry of the cured material can correspond to the desired cross-sectional geometry for the object 204. The parameters for operating the energy source 218 (e.g., energy intensity, energy dosage, exposure time, exposure pattern, exposure wavelength, energy density, power density) can be set based on instructions from a controller 224, as described in further detail below.

Once the object cross-section has been formed, the build platform 208 can be lowered by a predetermined amount to separate the cured material from the carrier film 210. The remaining curable material 206 can be carried by the carrier film 210 away from the build platform 208 and back toward the material source 216. The material source 216 can deposit additional curable material 206 onto the carrier film 210 and/or smooth the curable material 206 to re-form a uniform layer of curable material 206 on the carrier film 210. The curable material 206 can then be recirculated back to the build platform 208 to fabricate an additional layer of the object 204. This process can be repeated to iteratively build up individual object layers on the build platform 208 until the object 204 is complete. The object 204 and build platform 208 can then be removed from the system 200 for post-processing.

In some embodiments, the system 200 is used in a high temperature lithography process utilizing a highly viscous curable material 206 (e.g., a highly viscous resin). Accordingly, the printer assembly 202 can include one or more heat sources (heating plates, infrared lamps, etc.) for heating the curable material 206 to lower the viscosity to a range suitable for additive manufacturing. For example, the printer assembly 202 can include a first heat source 226a positioned against the segment of the carrier film 210 before the build platform 208, and a second heat source 226b positioned against the segment of the carrier film 210 after the build platform 208. Alternatively, or in combination, the printer assembly 202 can include heat sources at other locations.

The system 200 also includes a controller 224 (shown schematically) that is operably coupled to the printer assembly 202 and build platform 208 to control the operation thereof. The controller 224 can be or include a computing device including one or more processors and memory storing instructions for performing the additive manufacturing operations described herein. For example, the controller 224 can receive a digital data set (e.g., a three-dimensional model) representing the object 204 to be fabricated, determine a plurality of object cross-sections to build up the object 204 from the curable material 206, and can transmit instructions to the energy source 218 to output energy 220 to form the object cross-sections. As described above and in greater detail below, the controller 224 can control the energy application parameters of the energy source 218, such as the energy intensity, energy dosage, exposure time, exposure pattern, energy wavelength, and/or energy type of the energy 220 applied to the curable material 206. Optionally, the controller 224 can also determine and control other operational parameters, such as the positioning of the build platform 208 (e.g., height) relative to the carrier film 210, the movement speed and direction of the carrier film 210, the amount of curable material 206 deposited by the material source 216, the thickness of the material layer on the carrier film 210, and/or the amount of heating applied to the curable material 206.

Although FIG. 2 illustrates a representative example of a system 200 for additive manufacturing, this is not intended to be limiting, and the methods described herein can be implemented using other types of additive manufacturing systems, such as material jetting systems, binder jetting systems, material extrusion systems, powder bed fusion systems, sheet lamination systems, or directed energy deposition systems.

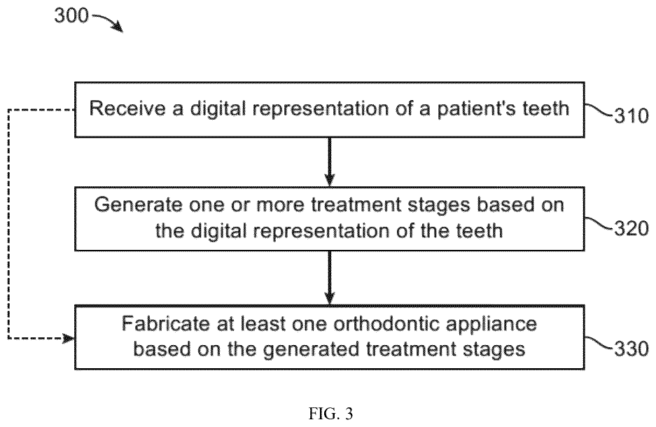

FIG. 3 illustrates a method 300 for digitally planning an orthodontic treatment and/or design or fabrication of an appliance, in accordance with embodiments. The method 300 can be applied to any of the treatment procedures described herein and can be performed by any suitable data processing system.

In step 310, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In step 320, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In step 330, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 3, design and/or fabrication of an orthodontic appliance, and perhaps a particular orthodontic treatment, may include use of a representation of the patient's teeth (e.g., receive a digital representation of the patient's teeth 310), followed by design and/or fabrication of an orthodontic appliance based on a representation of the patient's teeth in the arrangement represented by the received representation.

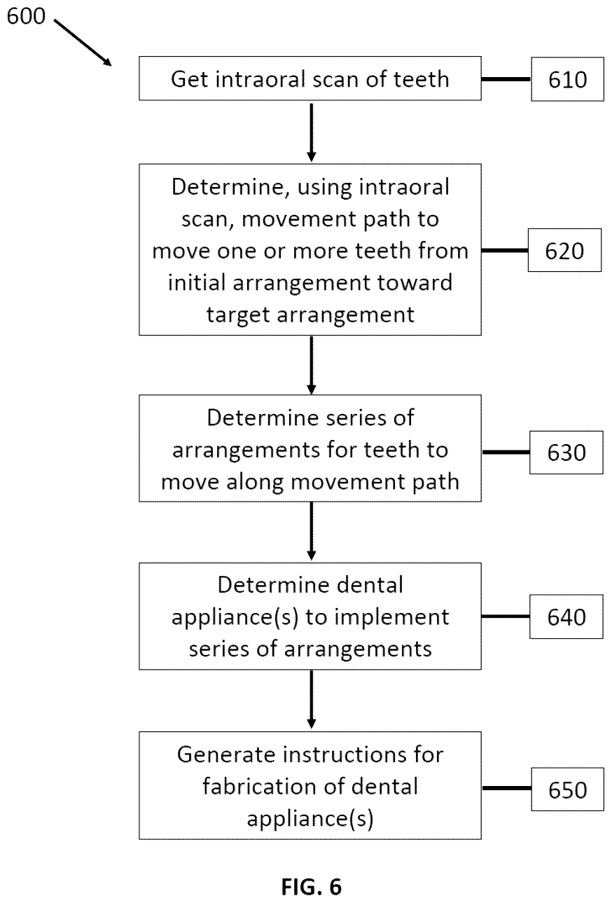

FIG. 6 illustrates a method 600 for designing an orthodontic appliance to be produced by direct fabrication, in accordance with embodiments. The method 600 can be applied to any embodiment of the orthodontic appliances described herein. Some or all of the steps of the method 600 can be performed by any suitable data processing system or device, e.g., one or more processors configured with suitable instructions.

In step 610, in intraoral scan is performed. In step 620, the information from the intraoral scan is used to determine a movement path to move one or more teeth from an initial arrangement to a target arrangement is determined. The initial arrangement can also be determined from a mold but is preferably obtained using an intraoral scan of the patient's teeth or mouth tissue (e.g., using wax bites, direct contact scanning, x-ray imaging, tomographic imaging, sonographic imaging, and other techniques for obtaining information about the position and structure of the teeth, jaws, gums and other orthodontically relevant tissue). From the obtained data, a digital data set can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, step 630 is used to determine a series of arrangements for teeth to move along (“a movement path”), which can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In step 640, a determination of dental appliance(s) to implement the movement path is made. The determination is made using knowledge related to a force system that can be used to produce movement of one or more teeth along the movement path. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully formed suture. Thus, in juvenile patients and others without fully closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

Accordingly, an optional additional step for 640 may be determining a configuration of an arch or palate expander design for an orthodontic appliance to produce the force system. Determination of the arch or palate expander design, appliance geometry, material composition, and/or properties can be performed using a treatment or force application simulation environment. A simulation environment can include, e.g., computer modeling systems, biomechanical systems, or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from several vendors can be used, including finite element analysis packages from ANSYS, Inc., of Canonsburg, PA, and SIMULIA(Abaqus) software products from Dassault Systèmes of Waltham, MA.

Optionally, one or more arch or palate expander designs can be selected for testing or force modeling. As noted above, a desired tooth movement, as well as a force system required or desired for eliciting the desired tooth movement, can be identified. Using the simulation environment, a candidate arch or palate expander design can be analyzed or modeled for determination of an actual force system resulting from use of the candidate appliance. One or more modifications can optionally be made to a candidate appliance, and force modeling can be further analyzed as described, e.g., to iteratively determine an appliance design that produces the desired force system.

In step 650, instructions for fabrication of the orthodontic appliance optionally incorporating an arch or palate expander design are generated. The instructions can be configured to control a fabrication system or device to produce the orthodontic appliance with the specified arch or palate expander design. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming.

Method 600 may comprise additional steps: 1) The upper arch and palate of the patient is scanned intraorally to generate three-dimensional data of the palate and upper arch; 2) The three-dimensional shape profile of the appliance is determined to provide a gap and teeth engagement structures as described herein.

Although the above steps show a method 600 of designing an orthodontic appliance in accordance with some embodiments, a person of ordinary skill in the art will recognize some variations based on the teaching described herein. Some of the steps may comprise sub-steps. Some of the steps may be repeated as often as desired. One or more steps of the method 600 may be performed with any suitable fabrication system or device, such as the embodiments described herein. Some of the steps may be optional, and the order of the steps can be varied as desired.

### On-Track Treatment

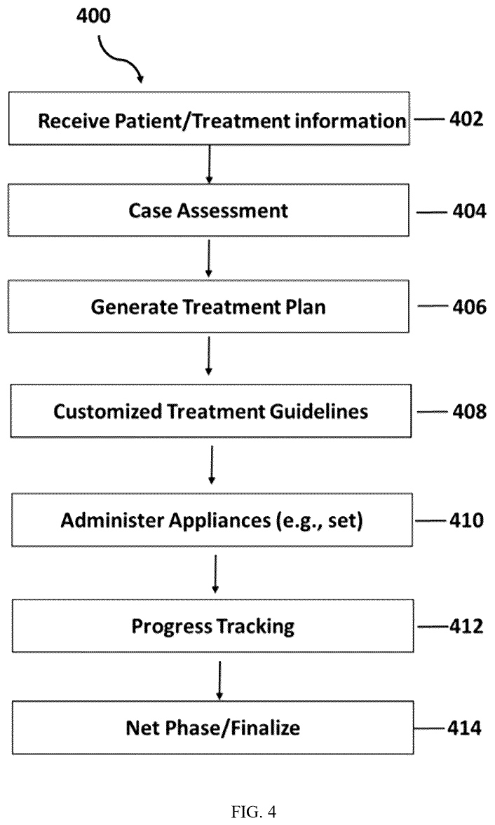

Referring to FIG. 4, a process 400 according to the present disclosure is illustrated. Individual embodiments of the process are discussed in further detail below. The process includes receiving information regarding the orthodontic condition of the patient and/or treatment information (402), generating an assessment of the case (404), and generating a treatment plan for repositioning a patient's teeth (406). Briefly, a patient/treatment information includes data comprising an initial arrangement of the patient's teeth, which includes obtaining an impression or scan of the patient's teeth prior to the onset of treatment and can further include identification of one or more treatment goals selected by the practitioner and/or patient. A case assessment can be generated (404) to assess the complexity or difficulty of moving the particular patient's teeth in general or specifically corresponding to identified treatment goals and may further include practitioner experience and/or comfort level in administering the desired orthodontic treatment. In some cases, however, the assessment can include simply identifying treatment options (e.g., appointment planning, progress tracking, etc.) that are of interest to the patient and/or practitioner. The information and/or corresponding treatment plan includes identifying a final or target arrangement of the patient's teeth that is desired, as well as a plurality of planned successive or intermediary tooth arrangements for moving the teeth along a treatment path from the initial arrangement toward the selected final or target arrangement.

The process further includes generating customized treatment guidelines (408). The treatment plan may include multiple phases of treatment, with a customized set of treatment guidelines generated that correspond to a phase of the treatment plan. The guidelines can include detailed information on timing and/or content (e.g., specific tasks) to be completed during a given phase of treatment and can be of sufficient detail to guide a practitioner, including a less experienced practitioner or practitioner relatively new to the orthodontic treatment process, through the phase of treatment. Since the guidelines are designed to specifically correspond to the treatment plan and provide guidelines on activities specifically identified in the treatment information and/or generated treatment plan, the guidelines can be customized. The customized treatment guidelines are then provided to the practitioner to help instruct the practitioner as how to deliver a given phase of treatment. As set forth above, appliances can be generated based on the planned arrangements and can be provided to the practitioner and ultimately administered to the patient (410). The appliances can be provided and/or administered in sets or batches of appliances, such as 2, 3, 4, 5, 6, 7, 8, 9, or more appliances, but are not limited to any administrative scheme. Appliances can be provided to the practitioner concurrently with a given set of guidelines, or appliances and guidelines can be provided separately.

After the treatment according to the plan begins and following administration of appliances to the patient, treatment progress tracking, e.g., by teeth matching, is done to assess a current and actual arrangement of the patient's teeth compared to a planned arrangement (412). If the patient's teeth are determined to be “on-track” and progressing according to the treatment plan, then treatment progresses as planned and treatment progresses to the next stage of treatment (414). If the patient's teeth have substantially reached the initially planned final arrangement, then treatment progresses to the final stages of treatment (414). Where the patient's teeth are determined to be tracking according to the treatment plan, but have not yet reached the final arrangement, the next set of appliances can be administered to the patient.

The threshold difference values of a planned position of teeth to actual positions selected as indicating that a patient's teeth have progressed on-track are provided below in TABLE 3. Ifa patient's teeth have progressed at or within the threshold values, the progress is on-track. Ifa patient's teeth have progressed beyond the threshold values, the progress is off-track.

TABLE 3

| Type Movement | Difference Actual/Planned | |
|---|---|---|
| Rotations | | |
| Upper Central Incisors | 9 | degrees |
| Upper Lateral Incisors | 11 | degrees |
| Lower Incisors | 11 | degrees |
| Upper Cuspids | 11 | degrees |
| Lower Cuspids | 9.25 | degrees |
| Upper Bicuspids | 7.25 | degrees |
| Lower First Bicuspid | 7.25 | degrees |
| Lower Second Bicuspid | 7.25 | degrees |
| Molars | 6 | degrees |
| Extrusion | | |
| Anterior | 0.75 | mm |
| Posterior | 0.75 | mm |
| Intrusion | | |
| Anterior | 0.75 | mm |
| Posterior | 0.75 | mm |
| Angulation | | |
| Anterior | 5.5 | degrees |
| Posterior | 3.7 | degrees |
| Inclination | | |
| Anterior | 5.5 | degrees |
| Posterior | 3.7 | degrees |
| Translation | | |
| BL Anterior | 0.7 | mm |
| BL Posterior Cuspids | 0.9 | mm |
| MD Anterior | 0.45 | mm |
| MD Cuspids | 0.45 | mm |
| MD Posterior | 0.5 | mm |

The patient's teeth are determined to be on track by comparison of the teeth in their current positions with teeth in their expected or planned positions, and by confirming the teeth are within the parameter variance disclosed in TABLE 1. If the patient's teeth are determined to be on track, then treatment can progress according to the existing or original treatment plan. For example, a patient determined to be progressing on track can be administered one or more subsequent appliances according to the treatment plan, such as the next set of appliances. Treatment can progress to the final stages and/or can reach a point in the treatment plan where bite matching is repeated for a determination of whether a patient's teeth are progressing as planned or if the teeth are off track.

In some embodiments, as further disclosed herein, this disclosure provides methods of treating a patient using a 3D printed orthodontic appliance. As a non-limiting example, orthodontic appliances comprising crystalline domains, polymer crystals, and/or materials that can form crystalline domains or polymer crystals can be 3D printed and used to reposition a patient's teeth. In certain embodiments, the method of repositioning a patient's teeth (or, in some embodiments, a singular tooth) comprises: generating a treatment plan for the patient, the plan comprising a plurality of intermediate tooth arrangements for moving teeth along a treatment path from an initial arrangement toward a final arrangement; producing a 3D printed orthodontic appliance; and moving on-track, with the orthodontic appliance, at least one of the patient's teeth toward an intermediate arrangement or a final tooth arrangement. In some embodiments, producing the 3D printed orthodontic appliance uses the crystallizable resins disclosed further herein. On-track performance can be determined, e.g., from TABLE 1, above.

In some embodiments, the method further comprises tracking the progression of the patient's teeth along the treatment path after administration of the orthodontic appliance. In certain embodiments, the tracking comprises comparing a current arrangement of the patient's teeth to a planned arrangement of the teeth. As a non-limiting example, following the initial administration of the orthodontic appliance, a period of time passes (e.g., two weeks), a comparison of the now-current arrangement of the patient's teeth (i.e., at two weeks of treatment) can be compared with the teeth arrangement of the treatment plan. In some embodiments, the progression can also be tracked by comparing the current arrangement of the patient's teeth with the initial configuration of the patient's teeth. The period of time can be, for example, greater than 3 days, greater than 4 days, greater than 5 days, greater than 6 days, greater than 7 days, greater than 8 days, greater than 9 days, greater than 10 days, greater than 11 days, greater than 12 days, greater than 13 days, greater than 2 weeks, greater than 3 weeks, greater than 4 weeks, or greater than 2 months. In some embodiments, the period can be from at least 3 days to at most 4 weeks, from at least 3 days to at most 3 weeks, from at least 3 days to at most 2 weeks, from at least 4 days to at most 4 weeks, from at least 4 days to at most 3 weeks, or from at least 4 days to at most 2 weeks. In certain embodiments, the period can restart following the administration of a new orthodontic appliance.

In some embodiments, greater than 50%, greater than 55%, greater than 60%, greater than 65%, greater than 70%, greater than 75%, greater than 80%, greater than 85%, greater than 90%, greater than 91%, greater than 92%, greater than 93%, greater than 94%, greater than 95%, greater than 96%, greater than 97%, greater than 98%, or greater than 99% of the patient's teeth are on track with the treatment plan after a period of time of using an orthodontic appliance as disclosed further herein. In some embodiments, the period is 3 days, 4 days, 5 days, 6 days, 7 days, 8 days, 9 days, 10 days, 11 days, 12 days, 13 days, 2 weeks, 3 weeks, 4 weeks, or greater than 4 weeks.

As disclosed further herein, orthodontic appliances disclosed herein have advantageous properties, such as increased durability, and an ability to retain resilient forces to a patient's teeth for a prolonged period. In some embodiments of the method disclosed above, the 3D printed orthodontic appliance has a retained repositioning force (i.e., the repositioning force after the orthodontic appliance has been applied to or worn by the patient over a period of time), and the retained repositioning force to at least one of the patient's teeth after the period of time is at least 10%, at least 20%, at least 30%, at least 40%, at least 50%, at least 55%, at least 60%, at least 65%, at least 70%, at least 75%, at least 80%, at least 85%, at least 90%, at least 95%, at least 96%, at least 97%, at least 98%, or at least 99% of the repositioning force initially provided to the at least one of the patient's teeth (i.e., with initial application of the orthodontic appliance). In some embodiments, the period is 1 day, 2 days, 3 days, 4 days, 5 days, 6 days, 7 days, 8 days, 9 days, 10 days, 11 days, 12 days, 13 days, 2 weeks, 3 weeks, 4 weeks, or greater than 4 weeks. In some embodiments, the repositioning force applied to at least one of the patient's teeth is present for a time period of less than 24 hours, from about 24 hours to about 2 months, from about 24 hours to about 1 month, from about 24 hours to about 3 weeks, from about 24 hours to about 14 days, from about 24 hours to about 7 days, from about 24 hours to about 3 days, from about 3 days to about 2 months, from about 3 days to about 1 month, from about 3 days to about 3 weeks, from about 3 days to about 14 days, from about 3 days to about 7 days, from about 7 days to about 2 months, from about 7 days to about 1 month, from about 7 days to about 3 weeks, from about 7 days to about 2 weeks, or greater than 2 months. In some embodiments, the repositioning force applied to at least one of the patient's teeth is present for about 24 hours, for about 3 days, for about 7 days, for about 14 days, for about 2 months, or for more than 2 months.

In some embodiments, the orthodontic appliances disclosed herein can provide on-track movement of at least one of the patient's teeth. On-track movement has been described further herein, e.g., at TABLE 1. In some embodiments, the orthodontic appliances disclosed herein can be used to achieve on-track movement of at least one of the patient's teeth to an intermediate tooth arrangement. In some embodiments, the orthodontic appliances disclosed herein can be used to achieve on-track movement of at least one of the patient's teeth to a final tooth arrangement.

In some embodiments, prior to moving, with the orthodontic appliance, at least one of the patient's teeth toward an intermediate arrangement or a final tooth arrangement, the orthodontic appliance has characteristics which are retained following the use of the orthodontic appliance. In some embodiments, prior to the moving step, the orthodontic appliance comprises a first flexural modulus. In certain embodiments, after the moving step, the orthodontic appliance comprises a second flexural modulus. In some embodiments, the second flexural modulus is at least 99%, at least 98%, at least 97%, at least 96%, at least 95%, at least 94%, at least 93%, at least 92%, at least 91%, at least 90%, at least 85%, at least 80%, at least 75%, at least 70%, at least 65%, at least 60%, at least 50%, or at least 40% of the first flexural modulus. In some embodiments, the second flexural modulus is greater than 50% of the first flexural modulus. In some embodiments, this comparison is performed following a period of time in which the appliance is applied. In some embodiments, the period is 3 days, 4 days, 5 days, 6 days, 7 days, 8 days, 9 days, 10 days, 11 days, 12 days, 13 days, 2 weeks, 3 weeks, 4 weeks, or greater than 4 weeks.

In some embodiments, prior to the moving step, the orthodontic appliance comprises a first elongation at break. In certain embodiments, after the moving step, the orthodontic appliance comprises a second elongation at break. In some embodiments, the second elongation at break is at least 99%, at least 98%, at least 97%, at least 96%, at least 95%, at least 94%, at least 93%, at least 92%, at least 91%, at least 90%, at least 85%, at least 80%, at least 75%, at least 70%, at least 65%, at least 60%, at least 50%, or at least 40% of the first elongation at break. In some embodiments, the second elongation at break is greater than 50% of the first elongation at break. In some embodiments, this comparison is performed following a period in which the appliance is applied. In some embodiments, the period is 3 days, 4 days, 5 days, 6 days, 7 days, 8 days, 9 days, 10 days, 11 days, 12 days, 13 days, 2 weeks, 3 weeks, 4 weeks, or greater than 4 weeks.

As provided herein, the methods disclosed can use the orthodontic appliances further disclosed herein. The orthodontic appliances can be directly fabricated using, e.g., the crystallizable resins disclosed herein. In certain embodiments, the direct fabrication comprises cross-linking the crystallizable resin.

The appliances formed from the crystallizable resins disclosed herein provide improved durability, strength, and flexibility, which in turn improve the rate of on-track progression in treatment plans. In some embodiments, greater than 60%, greater than 70%, greater than 80%, greater than 90%, or greater than 95% of patients treated with the orthodontic appliances disclosed herein (e.g., an aligner) are classified as on-track in a given treatment stage. In certain embodiments, greater than 60%, greater than 70%, greater than 80%, greater than 90%, or greater than 95% of patients treated with the orthodontic appliances disclosed herein (e.g., an aligner) have greater than 50%, greater than 55%, greater than 60%, greater than 65%, greater than 70%, greater than 75%, greater than 80%, greater than 85%, greater than 90%, or greater than 95% of their tooth movements classified as on-track.

As disclosed further herein, the cured polymeric material contains favorable characteristics that, at least in part, stem from the presence of polymeric crystals. These cured polymeric materials can have increased resilience to damage, can be tough, and can have decreased water uptake when compared to similar polymeric materials. The cured polymeric materials can be used for devices within the field of orthodontics, as well as outside the field of orthodontics. For example, the cured polymeric materials disclosed herein can be used to make devices for use in aerospace applications, automobile manufacturing, the manufacture of prototypes, and/or devices for use in durable parts production.

### Experimental Methods

All chemicals were purchased from commercial sources and were used without further purification, unless otherwise stated.

¹H NMR and ¹³C NMR spectra were recorded on a BRUKER AC-E-200 FT-NMR spectrometer or a BRUKER Avance DRX-400 FT-NMR spectrometer. The chemical shifts are reported in ppm (s: singlet, d: doublet, t: triplet, q: quartet, m: multiplet). The solvents used were deuterated chloroform (CDCl₃, 99.5% deuteration) and deuterated DMSO (d₆-DMSO, 99.8% deuteration).

In some embodiments, the stress relaxation of a material or device can be measured by monitoring the time-dependent stress resulting from a steady strain. The extent of stress relaxation can also depend on the temperature, relative humidity and other applicable conditions (e.g., presence of water). In embodiments, the test conditions for stress relaxation are a temperature of 37±2° C. at 100% relative humidity or a temperature of 37±2° C. in water.

The dynamic viscosity of a fluid indicates its resistance to shearing flows. The SI unit for dynamic viscosity is the Poiseuille (Pa-s). Dynamic viscosity is commonly given in units of centipoise, where 1 centipoise (cP) is equivalent to 1 mPa·s. Kinematic viscosity is the ratio of the dynamic viscosity to the density of the fluid; the SI unit is m²/s. Devices for measuring viscosity include viscometers and rheometers. For example, an MCR 301 rheometer from Anton Paar may be used for rheological measurement in rotation mode (PP-25, 50 s-1, 50-115° C., 3° C./min).

Determining the water content when fully saturated at use temperature can comprise exposing the polymeric material to 100% humidity at the use temperature (e.g., 40° C.) for a period of 24 hours, then determining water content by methods known in the art, such as by weight.

In some embodiments, the presence of a crystalline phase and an amorphous phase provide favorable material properties to the polymeric materials. Property values of the cured polymeric materials can be determined, for example, by using the following methods:
stress relaxation properties can be assessed using an RSA-G2 instrument from TA Instruments, with a 3-point bending, according to ASTM D790; for example, stress relaxation can be measured at 30° C. and submerged in water, and reported as the remaining load after 24 hours, as either the percent (%) of initial load, and/or in MPa;
storage modulus can be measured at 37° C. and is reported in MPa;
T_{g} of the cured polymeric material can be assessed using dynamic mechanical analysis (DMA) and is provided herein as the tan 8 peak;
tensile modulus, tensile strength, elongation at yield and elongation at break can be assessed according to ISO 527-2 5B; and tensile strength at yield, elongation at break, tensile strength, and Young's modulus can be assessed according to ASTM D1708.

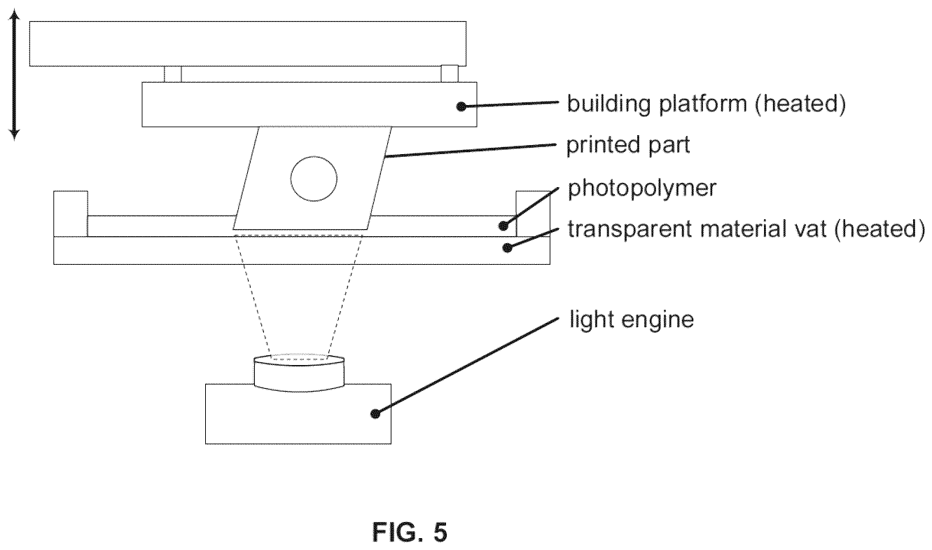

Additive manufacturing or 3D printing processes for generating a device herein (e.g., an orthodontic appliance) can be conducted using a Hot Lithography apparatus prototype from Cubicure (Vienna, Austria), which can substantially be configured as schematically shown in FIG. 5. In such cases, a photo-curable composition (e.g., resin) according to the present disclosure can be filled into the transparent material vat of the apparatus shown in FIG. 5, which vat can be heated to 90-110° C. The building platform can be heated to 90-110° C., too, and lowered to establish holohedral contact with the upper surface of the curable composition. By irradiating the composition with 375 nm UV radiation using a diode laser from Soliton, which can have an output power of 70 mW, which can be controlled to trace a predefined prototype design, and alternately raising the building platform, the composition can be cured layer by layer by a photopolymerization process according to the disclosure, resulting in a polymeric material according to present disclosure.

It will also be appreciated by those skilled in the art that in the process described herein the functional groups of starting materials or intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, include t-butoxycarbonyl, benzyloxycarbonyl, p-methoxybenzyl, trityl and the like.

Protecting groups may be added or removed in accordance with standard techniques, which are known to one skilled in the art and as described herein.

The use of protecting groups is described in detail in Greene, T. W. and P. G. M. Wuts, Greene's Protective Groups in Organic Synthesis (2006), 4^{th} Ed., Wiley. The protecting group may also be a polymer resin such as a Wang resin or a 2-chlorotrityl-chloride resin.

### EXAMPLES

The following examples are given for the purpose of illustrating various embodiments of the disclosure and are not meant to limit the present disclosure in any fashion. The present examples, along with the methods described herein are presently representative of some embodiments, are exemplary, and are not intended as limitations on the scope of the disclosure. Changes therein and other uses which are encompassed within the spirit of the disclosure as defined by the scope of the claims will occur to those skilled in the art.

A general synthetic scheme of multi-functional polymerizable compounds is illustrated below:

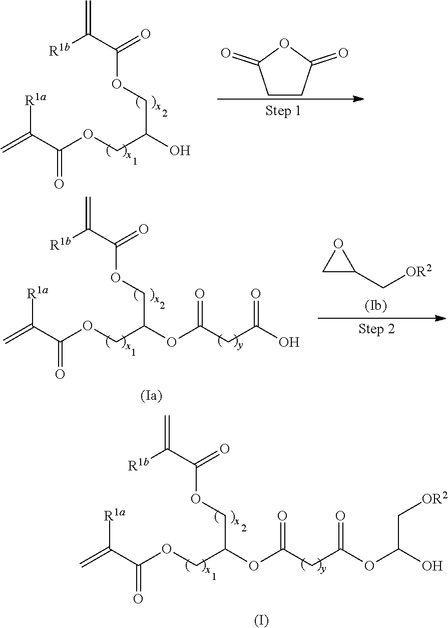

A reactant dimethacrylate is reacted with succinic anhydride to afford compound (Ia) via succinic anhydride ring opening reaction. The compound (Ia) is then reacted with an epoxide (Ib) to form a compound of Formula (I).

### Example 1

### Synthesis of 3-((1,3-Bis(Methacryloyloxy)Propan-2-Yl)Oxy)-3-Oxopropanoic Acid

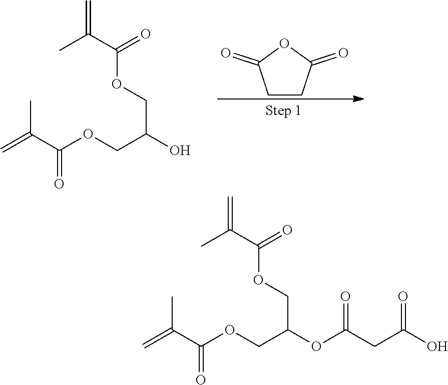 [SMILES: C=C(C)C(=O)OCC(O)COC(=O)C(=C)C ; O=C1CCC(=O)O1 ; C=C(C)C(=O)OCC(COC(=O)C(=C)C)OC(=O)CC(=O)O]

In a 1-L round-bottom flask equipped with a magnetic stir bar, glycerol dimethacrylate (100 g, 90.5% purity, 0.397 mol), succinic anhydride (46.0 g, 0.460 mol), and 4-dimethylaminopyridine (5.35 g, 43.8 mmol) were dissolved in dry tetrahydrofuran (438 mL). The reaction mixture was stirred at room temperature for 36 h. The reaction was confirmed to be complete when TLC (1:1 ethyl acetate/hexanes) showed the full conversion of glycerol dimethacrylate. The reaction mixture was concentrated in vacuo to remove THF and redissolved in ethyl acetate. The crude product was fully extracted into saturated NaHCO₃ (aq.). The combined aqueous layers were washed once with ethyl acetate. The combined aqueous layers were acidified to pH 3 using concentrated HCl. The final product was fully extracted into ethyl acetate. The combined organic layers were dried over MgSO₄, filtered, and concentrated in vacuo to result in 115 g (88% yield) of 3-((1,3-bis(methacryloyloxy)propan-2-yl)oxy)-3-oxopropanoic acid as a clear, colorless liquid.

### Example 2

### Synthesis of 3-((1-(Acryloyloxy)-3-(Methacryloyloxy)Propan-2-Yl)Oxy)-3-Oxopropanoic Acid

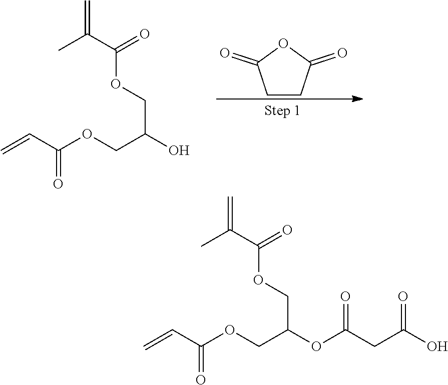 [SMILES: C=CC(=O)OCC(O)COC(=O)C(=C)C ; O=C1CCC(=O)O1 ; C=CC(=O)OCC(COC(=O)C(=C)C)OC(=O)CC(=O)O]

In a 500-mL round-bottom flask equipped with a magnetic stir bar, 3-(acryloyloxy)-2-hydroxypropyl methacrylate (50.0 g, 80% purity, 0.187 mol), succinic anhydride (24.5 g, 0.245 mol), and 4-dimethylaminopyridine (2.85 g, 23.3 mmol) were dissolved in dry tetrahydrofuran (233 mL). The reaction mixture was stirred at room temperature for 36 h. The reaction was confirmed to be complete when TLC (1:1 ethyl acetate/hexanes) showed the full conversion of 3-(acryloyloxy)-2-hydroxypropyl methacrylate. The reaction mixture was concentrated in vacuo to remove THF and redissolved in ethyl acetate. The crude product was fully extracted into saturated NaHCO₃ (aq.). The combined aqueous layers were washed once with ethyl acetate. The combined aqueous layers were acidified to pH 3 using concentrated HCl. The final product was fully extracted into ethyl acetate. The combined organic layers were dried over MgSO₄, filtered, and concentrated in vacuo to result in 19 g (32% yield) of 3-((1-(acryloyloxy)-3-(methacryloyloxy)propan-2-yl)oxy)-3-oxopropanoic acid as a clear, colorless liquid.

### Example 3

### Synthesis of 1,3-Bis(Methacryloyloxy)Propan-2-Yl (1-Hydroxy-2-(Methacryloyloxy)Ethyl) Malonate (Compound I-4)

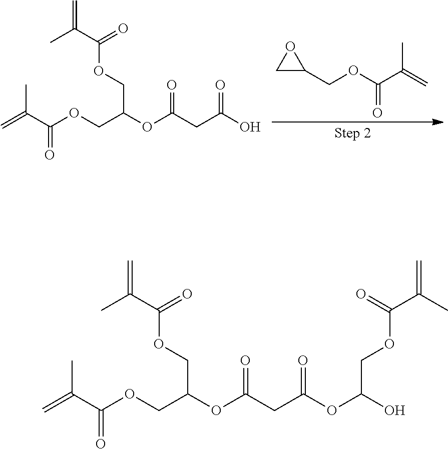

In a 250-mL round-bottom flask equipped with a magnetic stir bar, 3-((1,3-bis(methacryloyloxy)propan-2-yl)oxy)-3-oxopropanoic acid (50.0 g, 0.152 mol), glycidyl methacrylate (19.4 mL, 0.142 mol), 4-methoxyphenol (26.5 mg, 0.213 mmol), and triphenylphosphine (448 mg, 1.71 mmol) were combined. The reaction mixture was stirred at 80° C. for 20 h. The reaction was confirmed to be complete when TLC (1:1 ethyl acetate/hexanes) showed the full conversion of glycidyl methacrylate. The reaction mixture was diluted with ethyl acetate and washed with saturated NaHCO₃ (aq.). The organic layer was dried over MgSO₄, filtered, and concentrated in vacuo to result in 55 g (82% yield) of 1,3-bis(methacryloyloxy)propan-2-yl (1-hydroxy-2-(methacryloyloxy)ethyl) malonate as a clear, lightly yellow liquid.

### Example 4

### Synthesis of 2-(Acryloyloxy)-1-Hydroxyethyl (1,3-Bis(Methacryloyloxy)Propan-2-Yl) Malonate (Compound I-2)

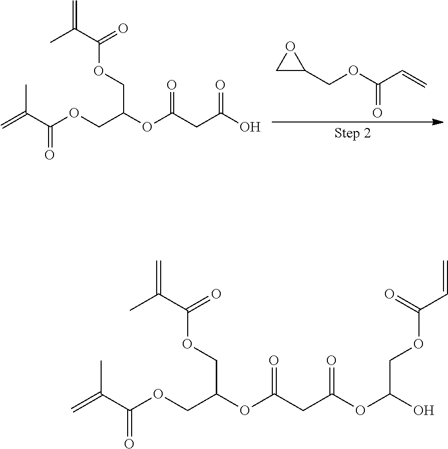

In a 250-mL round-bottom flask equipped with a magnetic stir bar, 3-((1,3-bis(methacryloyloxy)propan-2-yl)oxy)-3-oxopropanoic acid (22.0 g, 67.0 mmol), glycidyl acrylate (8.18 g, 63.8 mmol), 4-methoxyphenol (11.9 mg, 0.0957 mmol), and triphenylphosphine (201 mg, 0.766 mmol) were combined. The reaction mixture was stirred at 80° C. for 20 h. The reaction was confirmed to be complete when TLC (1:1 ethyl acetate/hexanes) showed the full conversion of glycidyl acrylate. The reaction mixture was diluted with ethyl acetate and washed with saturated NaHCO₃ (aq.). The organic layer was dried over MgSO₄, filtered, and concentrated in vacuo to result in 25 g (86% yield) of 2-(acryloyloxy)-1-hydroxyethyl (1,3-bis(methacryloyloxy)propan-2-yl) malonate as a clear, lightly yellow liquid.

### Example 5

### Synthesis of 2-((((5-(((4-(((3-(5,13-Bis((Methacryloyloxy)Methyl)-17-Methyl-16-Methylene-3,8,11-Trioxo-4,7,12,15-Tetraoxa-2-Azaoctadec-17-En-1-Yl)-3,5,5-Trimethylcyclohexyl)Carbamoyl)Oxy)Butoxy)Carbonyl)Amino)-1,3,3-Trimethylcyclohexyl)Methyl)Carbamoyl)Oxy)-3-(Methacryloyloxy)Propyl (1,3-Bis(Methacryloyloxy)Propan-2-Yl) Succinate (II-1)

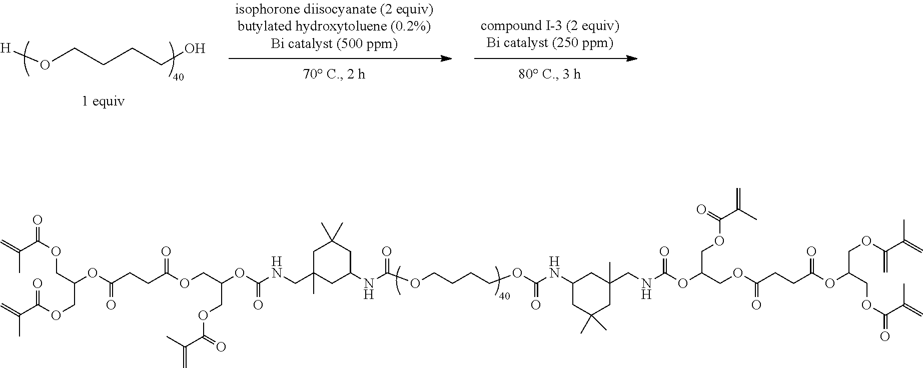

In a 1-L glass reactor equipped with an overhead stirrer, dried polytetrahydrofuran diol (Mn 2900) (87.0 g, 30.0 mmol), butylated hydroxytoluene (257 mg, 0.2 wt %), and isophorone diisocyanate (13.3 g, 60.0 mmol) were combined. The mixture was heated at 70° C. and stirred at 50 rpm until homogeneous and the temperature stabilized. Next, bismuth catalyst KAT 716-LA (64.3 mg, 500 ppm) was added to the mixture; to do this, a 0.4 g/mL solution of bismuth catalyst in ethyl acetate was prepared by dissolving KAT 716-LA (2 g) in ethyl acetate (5 mL), and 0.16 mL of the solution was added to the reaction mixture. The stirring speed was increased to 120 rpm. The reaction was allowed to proceed for 1.5 h (time point 1), after which an aliquot was removed from the mixture for FTIR analysis. The reaction was allowed to proceed for an additional 30 min or a total of 2 h (time point 2), after which a second aliquot was removed from the mixture for FTIR analysis. No observable difference in the FTIR spectra at time points 1 and 2 confirmed the complete conversion of the polymerdiol to the diisocyanate-end-functionalized prepolymer. At this time, compound I-3 (28.2 g, 60.0 mmol) and additional bismuth catalyst (0.08 mL of the 0.4 g/mL catalyst solution, 250 ppm) were added to the mixture. The temperature of the reaction was increased to 80° C. The reaction was allowed to proceed for 2 h, after which FTIR analysis of an aliquot of the mixture showed complete disappearance of the isocyanate signal. After an additional 1 h of reaction or a total of 3 h after the addition of compound I-3, the heat source wase turned off and the polymer melt allowed to cool to room temperature slowly to afford 2-((((5-(((4-(((3-(5,13-bis((methacryloyloxy)methyl)-17-methyl-16-methylene-3,8,11-trioxo-4,7,12,15-tetraoxa-2-azaoctadec-17-en-1-yl)-3,5,5-trimethylcyclohexyl)carbamoyl)oxy)butoxy)carbonyl)amino)-1,3,3-trimethylcyclohexyl)methyl)carbamoyl)oxy)-3-(methacryloyloxy)propyl (1,3-bis(methacryloyloxy)propan-2-yl) succinate.

### Example 6

Synthesis of Oligomers with Commercially Available Capping Agents

Oligomers with commercially available capping agents are described. (Hydroxyethyl)methacrylate (HEMA), glycerol-dimethacrylate (GDMA), and pentaerythritol trimethacrylate (PETM) are used as a capping agent (i.e., a methacrylate source) in place of compound I-3 in the synthetic procedure described in EXAMPLE 5 to provide oligomers with various end functionalities. HEMA, GDMA, and PETM have a functionality (i.e., a methacylate moiety) of 1, 2, and 3 (f=1, f=2, and f-3), respectively. Oligomers synthesized with HEMA, GDMA, and PETM have the following structures:

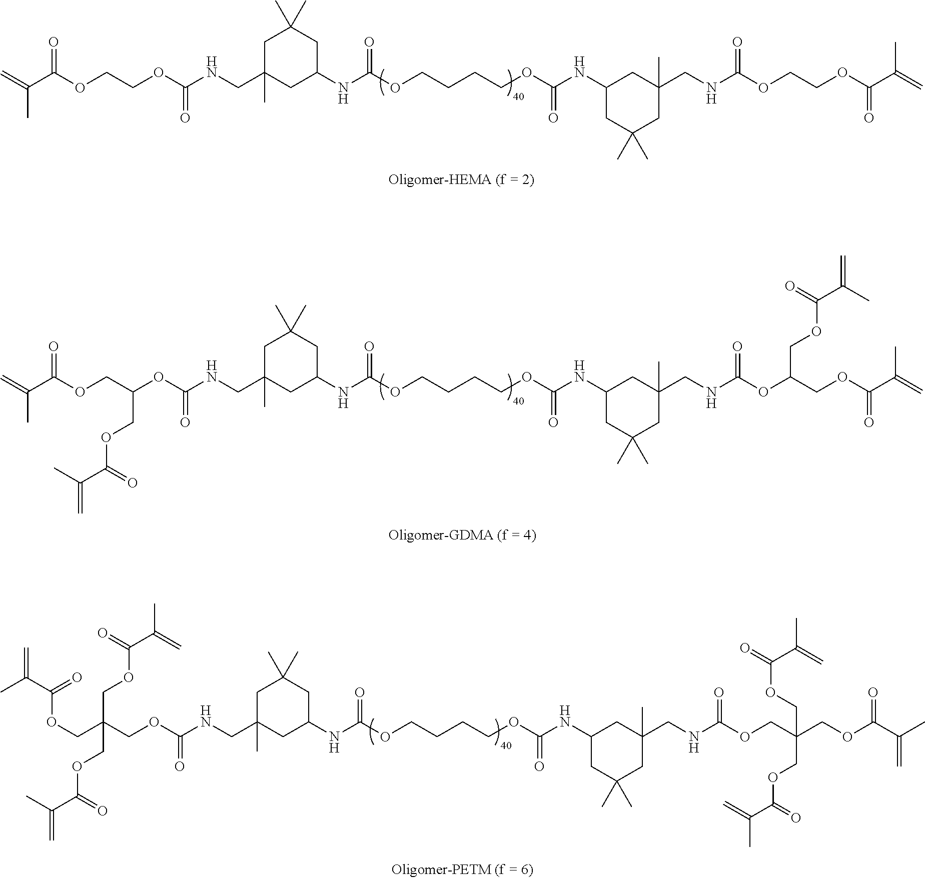

### Example 7

Mechanical Properties of a Polymer Synthesized with Oligomer Compound II-1

Mechanical properties of a polymer synthesized with oligomer compound II-1 is compared with polymers synthesized with oligomers-HEMA, -GDMA, and -PETM and the results are summarized in Table 4A below. Syringyl methacrylate (SMA) and phenylbis(2,4,6-trimethylbenzoyl)phosphine oxide (BAPO) were used in the polymer formulations.

Property values of the cured polymeric materials were determined by using the following methods:
tensile modulus, elongation at break and elongation at yield, and yield stress were assessed according to ASTM D1708;
T_{g} of the cured polymeric material was assessed using dynamic mechanical analysis (DMA) and is provided herein as the tan 8 peak;
stress relaxation properties were assessed using a DMA 850 instrument from TA Instruments with a 3-point bending clamp; for example, stress relaxation was measured at 30° C. and submerged in water, and reported as the remaining load after 24 hours in MPa.

TABLE 4A
A comparison chart of mechanical properties of casted films for polymers
synthesized with oligomer compound II-1 with oligomers synthesized
with commercially available methacrylates (casting temperature =
80° C., annealing temperature = 100° C., annealing time = 2 h)

| | | Control 1 | Control 2 | Control 3 | Polymer 1 |
|---|---|---|---|---|---|
| Composition | SMA (%) | 55 | 55 | 55 | 55 |
| | Oligomer-HEMA, | 45 | | | |
| | f = 2 (%) | | | | |
| | Oligomer-GDMA, | | 45 | | |
| | f = 4 (%) | | | | |
| | Oligomer-PETM, | | | 45 | |
| | f = 6 (%) | | | | |
| | Oligomer II-1, f = 6 | | | | 45 |
| | (%) | | | | |
| | BAPO (%) | 1 | 1 | 1 | 1 |
| Tensile | Young's Modulus | 791 | 1272 | 1424 | 1538 |
| | (MPa) | | | | |
| | Elongation at Break | 178 | 109 | 51 | 41 |
| | (%) | | | | |
| | Yield Stress (MPa) | --*- | 33.1 | 39.0 | 43.5 |
| | Elongation at Yield | --*- | 6.03 | 5.48 | 5.55 |
| | (%) | | | | |
| Thermomechanical | Tg - DMA (° C.) | 199 | 198 | >200 | 191 |
| Stress Relaxation | 24-Hr Flexural | 120 | 187 | 198 | 198 |
| | Modulus (MPa) | | | | |

*Control 1 material did not exhibit yield. Thus, yield stress and elongation at yield could not be measured

As expected, by increasing the oligomer end-functionality from f=2 (oligomer-HEMA) to f=6 (oligomer-PETM), the crosslinking density in the photocured material increased, resulting in a higher Young's modulus, yield stress, and 24-h flexural modulus, in addition to a lower elongation at break and elongation at yield. When the oligomer end-functionality was maintained at f=6 but the capping agent structure changed from PETM to compound I-4, the Young's modulus and yield stress continued to increase while elongation at break decreased, indicating that compound I-4 whose 3 methacrylate groups are more dispersed than those in PETM is an overall more reactive capping agent than PETM.

Oligomer II-1 and SMA were formulated in a few different compositions and tested for mechanical properties. The results are provided in Table 4B below.

TABLE 4B
A comparison chart of mechanical properties of casted
films for polymers synthesized with oligomer II-1 with various
compositions (casting temperature = 80° C., annealing
temperature = 100° C., annealing time = 2 h)

| | | Polymer | Polymer | Polymer |
|---|---|---|---|---|
| | | 1A | 1B | 1C |
| Composition | SMA (%) | 55 | 52 | 49 |
| | Oligomer II-1 (%) | 45 | 48 | 51 |
| | BAPO (%) | 1 | 1 | 1 |
| | Resin viscosity @ | 2.28 | 2.92 | 4.17 |
| | 60° C. (MPa) | | | |
| Tensile | Young's Modulus | 1538 | 1411 | 1261 |
| | (MPa) | | | |
| | Elongation at | 41 | 62 | 71 |
| | Break (%) | | | |
| | Yield Stress (MPa) | 43.5 | 39.7 | 36.0 |
| | Elongation at | 5.55 | 5.63 | 5.84 |
| | Yield (%) | | | |
| Thermomechanical | Tg - DMA (° C.) | 191 | 187 | 186 |
| Stress Relaxation | 24-Hr Flexural | 198 | 172 | 151 |
| | Modulus (MPa) | | | |

Table 4B illustrates that by adjusting the amount of oligomer II-1 in the formulation the (thermo)mechanical properties can be tuned. The same experiment was repeated with oligomer II-2 to study effects on having one less methyl group on each methacrylate group. The results are provided in Table 4C below.

TABLE 4C
A comparison chart of mechanical properties of casted
films for polymers synthesized with oligomer II-2 with various
compositions (casting temperature = 80° C., annealing
temperature = 100° C., annealing time = 2 h)

| | | Polymer | Polymer | Polymer |
|---|---|---|---|---|
| | | 2A | 2B | 2C |
| Composition | SMA (%) | 55 | 52 | 49 |
| | Oligomer II-2 (%) | 45 | 48 | 51 |
| | BAPO (%) | 1 | 1 | 1 |
| Tensile | Young's Modulus | 1462 | 1333 | 1169 |
| | (MPa) | | | |
| | Elongation at | 54 | 66 | 77 |
| | Break (%) | | | |
| | Yield Stress (MPa) | 41.2 | 37.7 | 33.4 |
| | Elongation at | 5.73 | 5.93 | 6.91 |
| | Yield (%) | | | |
| Thermomechanical | Tg - DMA (° C.) | 184 | 182 | 184 |
| Stress Relaxation | 24-Hr Flexural | 189 | 158 | 142 |
| | Modulus (MPa) | | | |

Compared to the all-methacrylate f=3 compound I-4, the 2:1 methacrylate/acrylate compound I-2 resulted in slightly lower Young's modulus, yield stress, and 24-hr flexural modulus and slightly higher elongation at break and elongation at yield.

Another sample was prepared using a polymerizable composition comprising a tetra-functional thiol and the same polycaprolactone dumbbell in a 10 mol % thiol to methacrylate functional mole percent ratio. The composition also included 1 wt % TPO-L. The mixture was heated at 70° C. and the dip and cure were performed as noted above. The formed polymer coating was still very slightly tacky immediately following the curing step. After cooling, the polymer coated article was not tacky. The resultant polymer coating was 50 microns thick as measured by a microscopy measurement of a crosscut section.

### Example 8

Mechanical Properties of a Polymer Synthesized with Oligomer Compound III-1

Oligomer III-1 and SMA were formulated in a few different compositions and tested for mechanical properties. The results are provided in Table 5 below. Syringyl methacrylate (SMA) and phenylbis(2,4,6-trimethylbenzoyl)phosphine oxide (BAPO) were used in the polymer formulations.

Property values of the cured polymeric materials were determined by using the following methods:
tensile modulus, elongation at break and elongation at yield, and yield stress were assessed according to ASTM D1708;
T_{g} of the cured polymeric material was assessed using dynamic mechanical analysis (DMA) and is provided herein as the tan 8 peak;
stress relaxation properties were assessed using a DMA 850 instrument from TA Instruments with a 3-point bending clamp; for example, stress relaxation was measured at 30° C. and submerged in water, and reported as the remaining load after 24 hours in MPa.

TABLE 5
A comparison chart of mechanical properties of casted
films for polymers synthesized with oligomer compound
III-1 with oligomers synthesized with commercially available
methacrylates (casting temperature = 80° C.,
annealing temperature = 100° C., annealing time = 2 h)

| | | Polymer 3A | Polymer 3B |
|---|---|---|---|
| Composition | SMA (%) | 53 | 50 |
| | Oligomer III-1, f = 6 (%) | 47 | 50 |
| | BAPO (%) | 1 | 1 |
| Tensile | Young's Modulus (MPa) | 1514 ± 48 | 1465 ± 36 |
| | Elongation at Break (%) | 6 ± 6 | 9 ± 7 |
| | Yield Stress (MPa) | 37.5 ± 9 | 39.4 ± 7.1 |
| | Elongation at Yield (%) | 4.1 ± 2.0 | 4.7 ± 1.9 |
| Stress Relaxation | 24-Hr Flexural Modulus | 249 | 230 |
| | (MPa) | | |

### Example 9

Mechanical Properties of a Polymer Synthesized with Oligomer Compound IV-1-IV-3

Oligomer IV-1-IV-3 and SMA were formulated in a few different compositions and tested for mechanical properties. The results are provided in Table 6A-6B below. Syringyl methacrylate (SMA) and phenylbis(2,4,6-trimethylbenzoyl)phosphine oxide (BAPO) were used in the polymer formulations.

Oligomer IV-1 and SMA were formulated in a few different compositions and tested for mechanical properties. The results are provided in Table 6A below.

| | | Polymer 4A | Polymer 4B |
|---|---|---|---|
| Composition | SMA (%) | 55 | 53 |
| | Oligomer IV-1, f = 6 (%) | 45 | 47 |
| | BAPO (%) | 1 | 1 |
| Tensile | Young's Modulus (MPa) | 1490 | 1400 |
| | Elongation at Break (%) | 50 | 79 |
| | Yield Stress (MPa) | 43.1 | 40.5 |
| Stress | 24-Hr Flexural | 205 | 184 |
| Relaxation | Modulus (MPa) | | |

A combination of oligomer IV-2 and SMA and oligomer IV-3 and SMA were formulated and tested for mechanical properties. The results are provided in Table 6B below.

| | | Polymer 4C | Polymer 4D |
|---|---|---|---|
| Composition | SMA (%) | 55 | 55 |
| | Oligomer IV-2, f = 4 (%) | 45 | |
| | Oligomer IV-3, f = 4 (%) | | 45 |
| | BAPO (%) | 1 | 1 |
| Tensile | Young's Modulus (MPa) | 1352 | 1357 |
| | Elongation at Break (%) | 44 | 48 |
| | Yield Stress (MPa) | 37.2 | 37.5 |

### Example 10

Mechanical Properties of a Polymer Synthesized with Oligomer Compound V-1

Oligomer V-1 and SMA were formulated and tested for mechanical properties. The results are provided in Table 7 below.

| | | Polymer 5 |
|---|---|---|
| Composition | SMA (%) | 53 |
| | Oligomer V-1, f = 6 (%) | 47 |
| | BAPO (%) | 1 |
| Tensile | Young's Modulus (MPa) | 1200 |
| | Elongation at Break (%) | 88 |
| | Yield Stress (MPa) | 43 |
| Stress Relaxation | 24-Hr Flexural Modulus (MPa) | 160 |

### Example 11

### Synthesis of in 4-(2-(4-(2-Hydroxy-2-Methylpropanoyl)Phenoxy)Ethoxy)-4-Oxobutanoic Acid

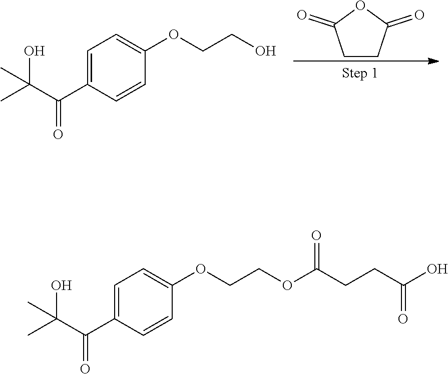 [SMILES: CC(C)(O)C(=O)c1ccc(OCCO)cc1 ; O=C1CCC(=O)O1 ; CC(C)(O)C(=O)c1ccc(OCCOC(=O)CCC(=O)O)cc1]

In a 500-mL round-bottom flask equipped with a magnetic stir bar, 2-hydroxy-1-(4-(2-hydroxyethoxy)phenyl)-2-methylpropan-1-one (41.9 g, 0.187 mol), succinic anhydride (24.5 g, 0.245 mol), and 4-dimethylaminopyridine (2.85 g, 23.3 mmol) were dissolved in dry tetrahydrofuran (233 mL). The reaction mixture was stirred at room temperature for 36 h. The reaction was confirmed to be complete when TLC (1:1 ethyl acetate/hexanes) showed the full conversion of 2-hydroxy-1-(4-(2-hydroxyethoxy)phenyl)-2-methylpropan-1-one. The reaction mixture was concentrated in vacuo to remove THF and redissolved in ethyl acetate. The crude product was fully extracted into saturated NaHCO₃ (aq.). The combined aqueous layers were washed once with ethyl acetate. The combined aqueous layers were acidified to pH 3 using concentrated HCl. The final product was fully extracted into ethyl acetate. The combined organic layers were dried over MgSO₄, filtered, and concentrated in vacuo to result in 4-(2-(4-(2-hydroxy-2-methylpropanoyl)phenoxy)ethoxy)-4-oxobutanoic acid.

### Example 12

### Synthesis of 1-2-(4-(2-Hydroxy-2-Methylpropanoyl)Phenoxy)Ethyl (1-Hydroxy-3-(Methacryloyloxy)Propan-2-Yl) Succinate (Compound VI-1)

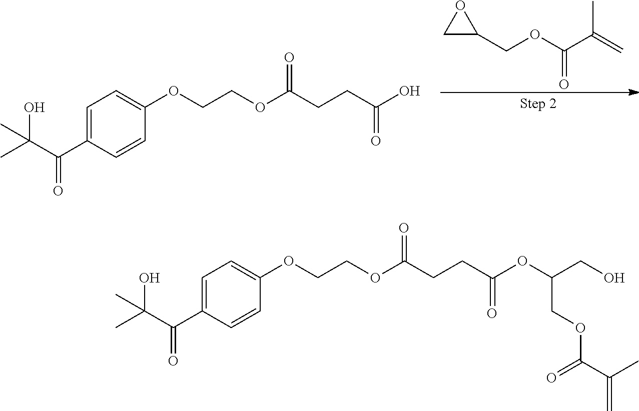

In a 250-mL round-bottom flask equipped with a magnetic stir bar, 3-(2-(4-(2-hydroxy-2-methylpropanoyl)phenoxy)ethoxy)-3-oxopropanoic acid (47.1 g, 0.152 mol), glycidyl methacrylate (19.4 mL, 0.142 mol), 4-methoxyphenol (26.5 mg, 0.213 mmol), and triphenylphosphine (448 mg, 1.71 mmol) were combined. The reaction mixture was stirred at 80° C. for 20 h. The reaction was confirmed to be complete when TLC (1:1 ethyl acetate/hexanes) showed the full conversion of glycidyl methacrylate. The reaction mixture was diluted with ethyl acetate and washed with saturated NaHCO₃ (aq.). The organic layer was dried over MgSO₄, filtered, and concentrated in vacuo to result in 2-(4-(2-hydroxy-2-methylpropanoyl)phenoxy)ethyl (1-hydroxy-3-(methacryloyloxy)propan-2-yl) succinate.

### Example 13

Mechanical Properties of a Polymer Synthesized with Oligomer Compound VII-1

Mechanical properties of a polymer synthesized with oligomer compound VII-1 are compared with polymers synthesized with oligomers-HEMA and -GDMA, and the results are summarized in Table 8 below. Syringyl methacrylate (SMA) and phenylbis(2,4,6-trimethylbenzoyl)phosphine oxide (BAPO) were used in the polymer formulations.

| | | Control 4 | Polymer 6 |
|---|---|---|---|
| Composition | SMA (%) | 53 | 53 |
| | Oligomer-HEMA, | 10 | |
| | f = 2 (%) | | |
| | Oligomer-GDMA, | 37 | 37 |
| | f = 4 (%) | | |
| | Oligomer VII-1, | | 10 |
| | f = 2 (%) | | |
| | BAPO (%) | 1 | 1 |
| Tensile | Young's Modulus (MPa) | 1103 | 1190 |
| | Elongation at Break (%) | 121 | 102 |
| | Yield Stress (MPa) | 30.0 | 31.9 |
| Stress | 24-Hr Flexural | 140 | 151 |
| Relaxation | Modulus (MPa) | | |

While Control 4 and Polymer 6 both contain 10% of a di-methacrylated oligomer with identical backbones, the dimethacrylated Oligomer VII-1 in Polymer 6 also contains 2 photoinitiator moieties, one at each oligomer chain end. Compared to Control 4, Polymer 6 has a higher Young's modulus, yield stress, and final flexural modulus and lower elongation at break. This indicates that Oligomer VII-1 with 2 methacrylates and 2 photoinitiator moieties behaves more like a multi-functional (f>2) oligomer crosslinker than it behaves like purely difunctional crosslinker Oligomer-HEMA.

1. A compound having the structure of Formula (I) or a salt thereof: 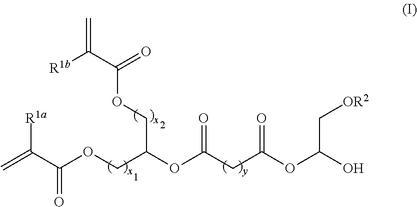
wherein:
R^{1a} and R^{1b} are, each independently, H, halogen, or C₁-C₆ alkyl;
R² is H, C₁-C₂₀ alkyl, —C(═O)C₁-C₂₀ alkyl, C₁-C₆ haloalkyl, acrylate, silyl ether, or a moiety comprising a photoinitiator; and
x₁, x₂, and y are, each independently, 1, 2 or 3.

2. A compound having the structure of Formula (VI) or a salt thereof: 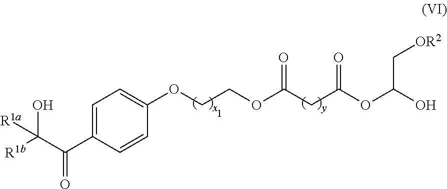
wherein:
R^{1a} and R^{1b} are, each independently, H, halogen, or C₁-C₆ alkyl;
R² is H, C₁-C₂₀ alkyl, —C(═O)C₁-C₂₀ alkyl, C₁-C₆ haloalkyl, acrylate, silyl ether, or a moiety comprising a photoinitiator; and
x₁ and y are, each independently, 1, 2 or 3.

3. The compound of claim 1, wherein R ^{1a} and R^{1b} are, each independently, H or C₁-C₃ alkyl.

4. (canceled)

5. The compound of claim 1, wherein R ^{1a} and R^{1b} are, each independently, H or —CH₃.

6. (canceled)

7. The compound of claim 1, wherein R² is acrylate or silyl ether.

8. The compound of claim 1, wherein the acrylate of R² has the following structure: 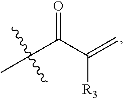
wherein R³ is H, halogen, or C₁-C₆ alkyl.

9. The compound of claim 1, wherein the silyl ether of R² has the following structure: 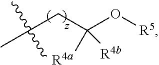
wherein:
R^{4a} and R^{4b} are, each independently H or C₁-C₆ alkyl;
R⁵ is H, C₁-C₆ alkyl, or silyl; and
z is an integer from 1 to 5.

10. The compound of claim 1 wherein C₁-C₆ haloalkyl of R² is C₂-C₄ haloalkyl.

11. The compound of claim 10, wherein of R² is 1,1,2,2-tetrafluoropropyl.

12. The compound of claim 1 wherein the photoinitiator has the following structure:

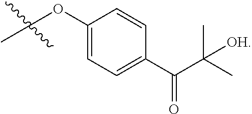 [SMILES: *Oc1ccc(C(=O)C(C)(C)O)cc1]

13. The compound of claim 1, wherein R² has one of the following structures:

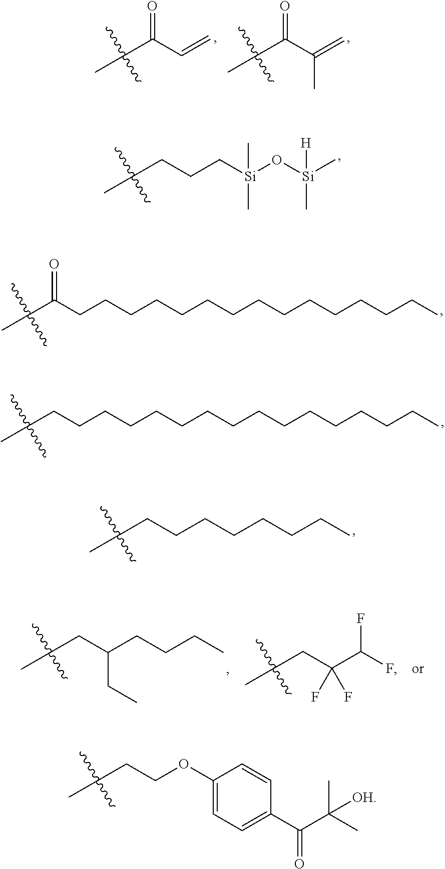

14. The compound of claim 1, wherein x₁ and x₂ are 1 and y is 2.

15. (canceled)

16. The compound of claim 1, wherein the compound has one of the following structures:

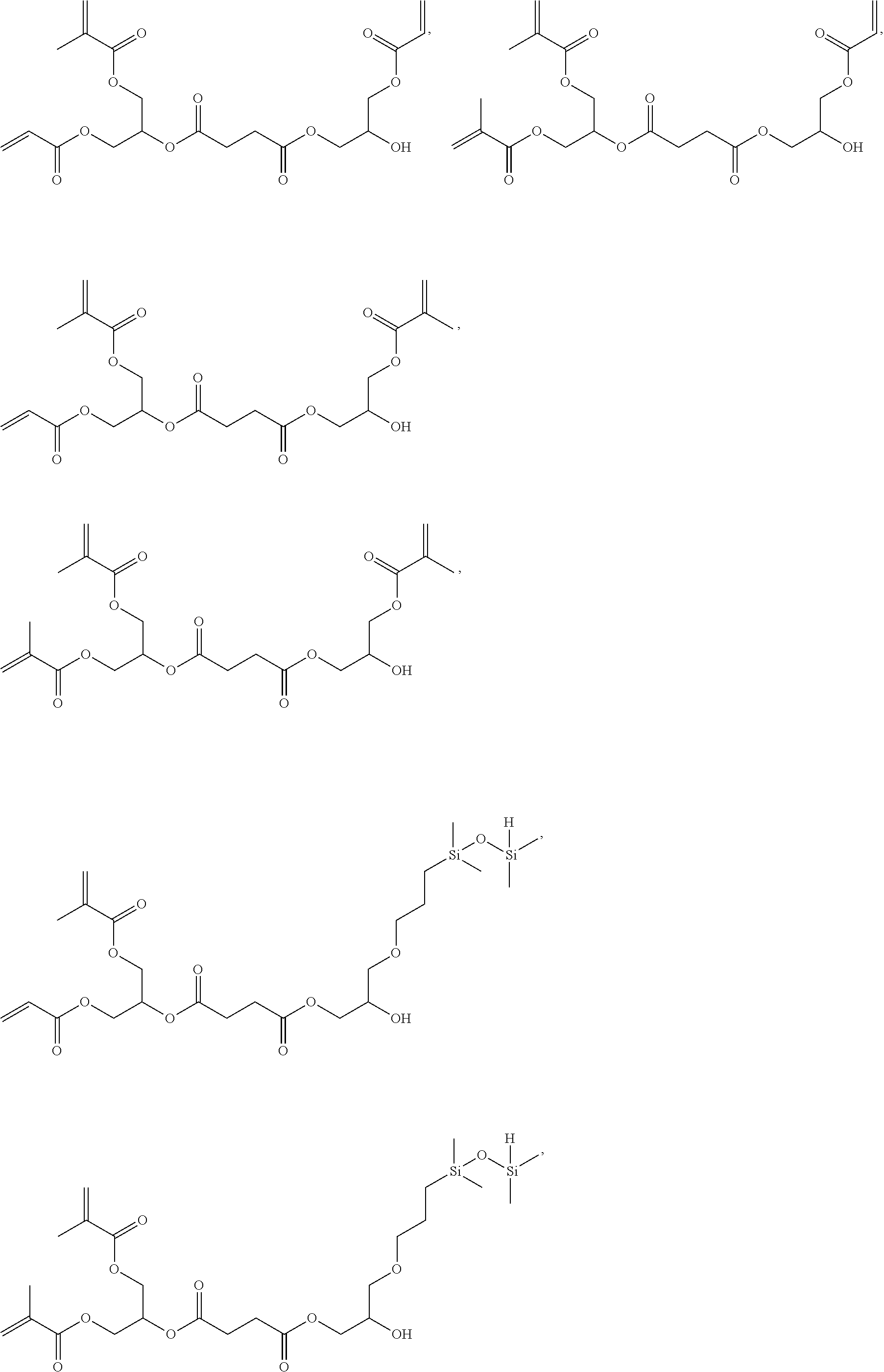

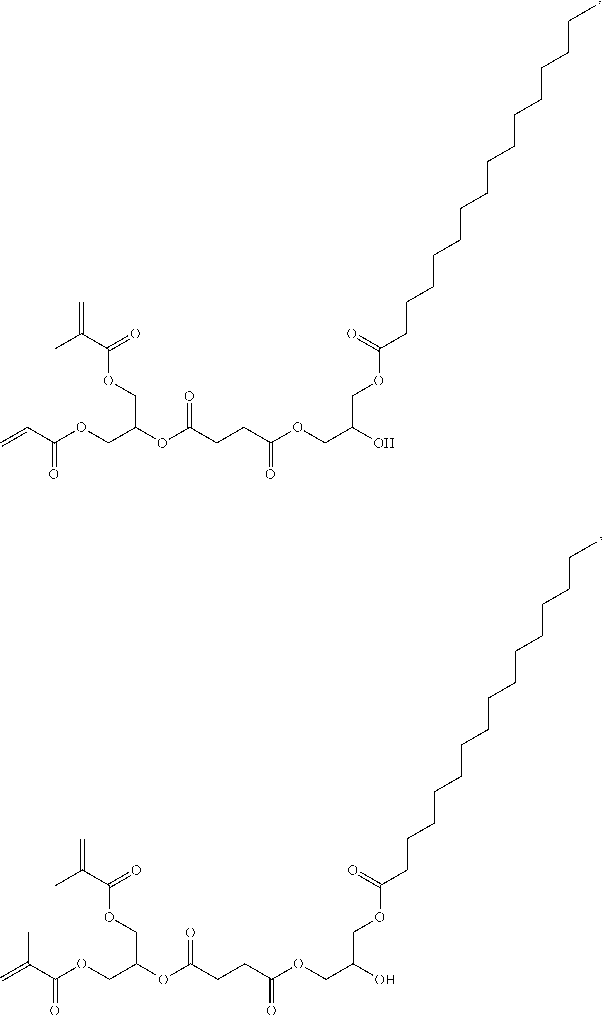

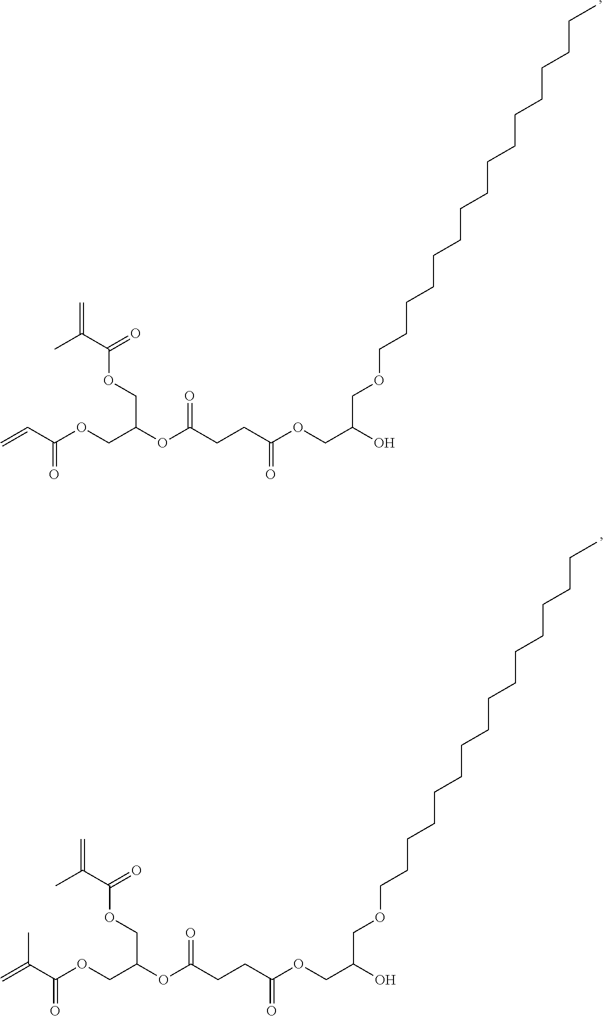

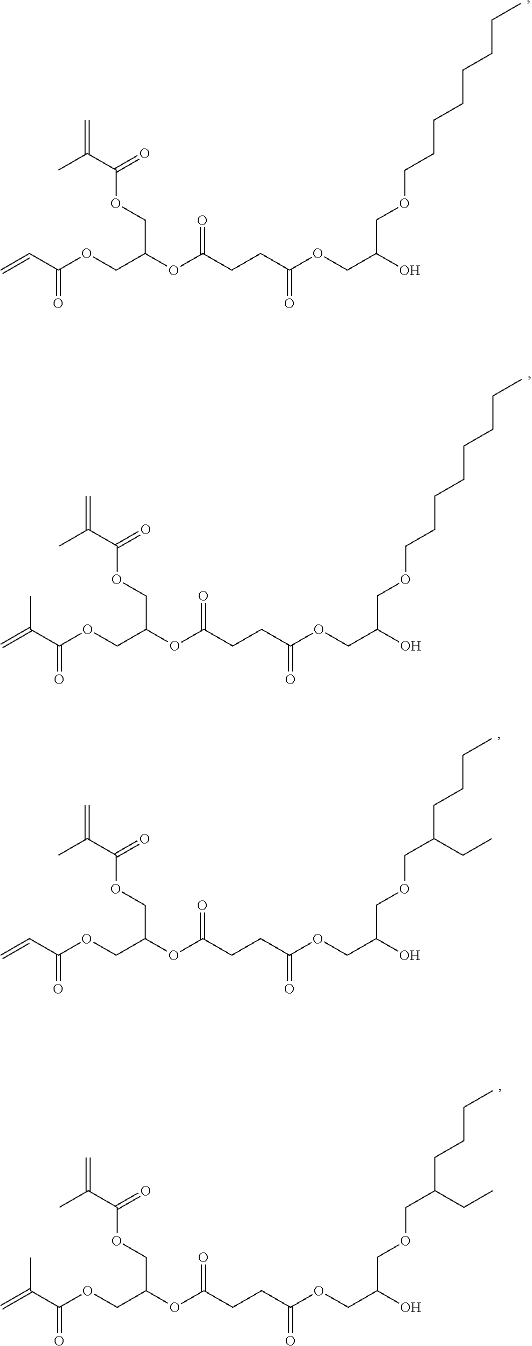

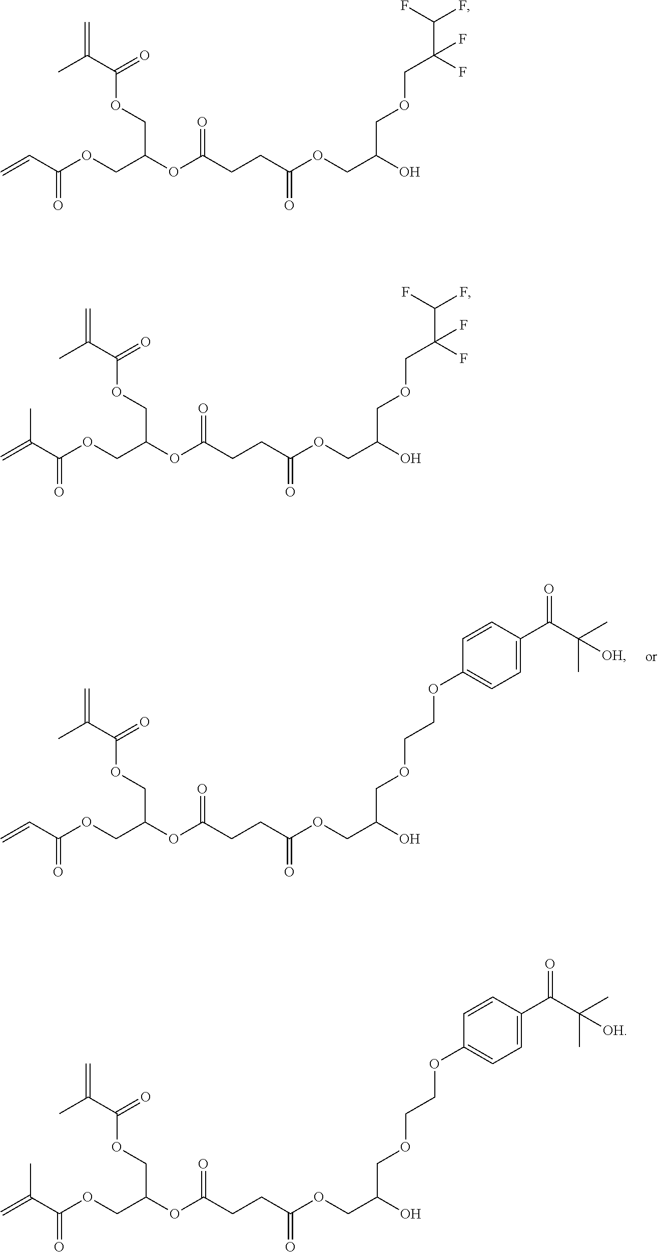 [SMILES: C=CC(=O)OCC(COC(=O)C(=C)C)OC(=O)CCC(=O)OCC(O)COCC(F)(F)C(F)F ; C=C(C)C(=O)OCC(COC(=O)C(=C)C)OC(=O)CCC(=O)OCC(O)COCC(F)(F)C(F)F ; C=CC(=O)OCC(COC(=O)C(=C)C)OC(=O)CCC(=O)OCC(O)COCCOc1ccc(C(=O)C(C)(C)O)cc1 ; "or" ; C=C(C)C(=O)OCC(COC(=O)C(=C)C)OC(=O)CCC(=O)OCC(O)COCCOc1ccc(C(=O)C(C)(C)O)cc1]

17. (canceled)

18. A composition comprising a compound of claim 1 and an oligomer comprising a glass transition temperature (T_{g}) of at most −10° C.

19-29. (canceled)

30. The composition of claim 18, wherein upon curing, the composition comprises at least one property selected from the group consisting of:
a Young's modulus at 1.7 mm/min of from 800 to 1500 megapascals (MPa);
a Young's modulus at 510 mm/min of from 1200 to 2000 MPa;
an elongation at break at 1.7 mm/min of from 60% to 200%;
an elongation at break at 510 mm/min of from 10% to 120%;
a yield stress at 1.7 mm/min of from 24 to 35 MPa;
a yield stress at 510 mm/min of from 35 to 55 MPa;
a 24 hour force of from 1.3 to 3.0 Newtons (N); and
a glass transition temperature of from 90 to 160° C.

31. A method for synthesizing an oligomer compound, the method comprising:
i) coupling the compound of claim 1 with an isophorone diisocyanate and a polytetrahydrofuran diol
ii) coupling the compound of claim 1 with an isophorone diisocyanate and a polyester diol; or
iii) the compound of claim 1 with an isophorone diisocyanate, a polyester diol, and polycarbonate diol.

32. (canceled)

33. A method for synthesizing the compound of claim 1, the method comprising reacting a compound of Formula (Ia) with an epoxide of Formula (Ib): 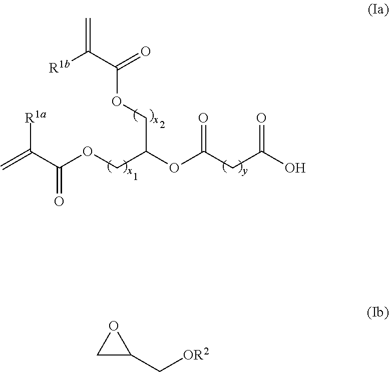
wherein:
R^{1a} and R^{1b} are, each independently, H, halogen, or C₁-C₆ alkyl;
R² is H, C₁-C₂₀ alkyl, —C(═O)C₁-C₂₀ alkyl, C₁-C₆ haloalkyl, acrylate, silyl ether, or a moiety comprising a photoinitiator; and
x₁, x₂, and y are each independently 1, 2, or 3.

34-89. (canceled)

90. A polymerizable composition comprising:
i) the oligomer of claim 1;
ii) a reactive diluent; and
iii) an initiator.

91-110. (canceled)

111. A polymer made from the polymerizable composition of claim 90.

112. A method for forming an article by additive manufacturing comprising: providing a first polymerizable composition;
exposing the first polymerizable composition to radiation;
polymerizing the first polymerizable composition layer-by-layer based on a predefined design, thereby polymerizing the first polymerizable composition to form a polymer;
fabricating the article with the polymer; and
contacting the article with a second polymerizable composition thereby forming a polymer coating, wherein the second polymerizable composition is the polymerizable composition of claim 90.

113-129. (canceled)
